(19) United States
(12) Patent Application Publication
(10) Pub. No.: US 2020/0010857 A1
(43) Pub. Date: Jan. 9, 2020

(54) RHODOCOCCUS AETHERIVORANS BCP1 AS CELL FACTORY FOR THE PRODUCTION OF INTRACELLULAR TELLURIUM AND/OR SELENIUM NANOSTRUCTURES (NANOPARTICLES OR NANORODS) UNDER AEROBIC CONDITIONS

(71) Applicants: UTI LIMITED PARTNERSHIP, Calgary (CA); ALMA MATER STUDIORUM - UNIVERSITY OF BOLOGNA, Bologna (IT)

(72) Inventors: Raymond TURNER, Calgary (CA); Davide ZANNONI, Bologna (IT); Alessandro PRESENTATO, Verona (IT); Elena PIACENZA, Verona (IT); Martina CAPPELLETTI, Bologna (IT); Max ANIKOVSKIY, Calgary (CA)

(21) Appl. No.: 16/468,401

(22) PCT Filed: Dec. 13, 2017
(86) PCT No.: PCT/CA2017/051512

§ 371 Date: Jun. 11, 2019

Related U.S. Application Data
(60) Provisional application No. 62/434,038, filed on Dec. 14, 2016.

(51) Int. Cl. C12P 1/04; C12P 3/00; C01B 19/00

(52) U.S. Cl. CPC C12P 1/04; C12P 3/00; B82Y 40/00; C01B 19/008; C01B 19/004

(57) ABSTRACT

The present disclosure relates generally to the production of tellurium and selenium nanostructures in bacteria. The nanostructures are unique in size, shape, length and stability.

### CROSS REFERENCE TO RELATED APPLICATION

This application claims priority to U.S. 62/434,038, filed on Dec. 14, 2016, the entire contents of which is hereby incorporated by reference.

### FIELD

The present disclosure relates generally to the production of tellurium nanostructures and selenium nanostructures in bacteria.

### BACKGROUND

Tellurium and selenium are useful in a wide ranges of industrial applications.

Tellurium (Te) was discovered by Franz-Joseph Müller von Reicheinstein in 1782, and in nature this element can be found in gold ores as association with metals, forming calaverite (AuTe₂), sylvanite (AgAuTe₄) and nagyagite [AuPb(Sb, Bi)Te₂₋₃S6]. Te is an element of the chalcogen family, belonging to the Group 16 of the periodic table along with oxygen (O), sulfur (S), selenium (Se), and the radioactive element polonium (Po). Additionally, it is defined as a metalloid due to its intermediate properties between metals and non-metals.

Selenium (Se) was discovered by Jöns Jacob Berzelius in 1817 as red-brown precipitate in association with sulfuric acid. It is naturally present in our earth crust as rare element in native rocks and ores, soils, sediments or as association in rare minerals (e.g., crooksite and calusthalite), with concentration ranging from 0.01 to 1200 mg/kg. Moreover, Se is an essential micronutrient for living systems as part of the structure of important enzymes, such as glutathione peroxidases and thioredoxin reductases]. In humans, it has multiple beneficial effects due to its presence in the substituted amino acid cysteine as seleno-cysteine, leading to the regulation of at least 25 selenoproteins.

There remains a need for methods for the productions of tellurium nanostructures and selenium nanostructures.

### SUMMARY

In one aspect there is described a method of producing tellurium nanostructures, comprising: culturing Rhodococcus aetherivorans (BCP1) bacteria in a medium comprising tellurite.

In one example, said culturing comprises pre-culturing said bacteria in said medium to generate a pre-culture, followed by culturing a portion of said pre-culture in said medium comprising tellurite to form a first culture.

In one example, further comprising a culturing a portion of said first culture in said medium comprising tellurite to form a second culture.

In one example, said culturing is performed under aerobic conditions.

In one example, wherein said culturing is performed under aerobic conditions at temperatures 20-40° C.

In one example, wherein said tellurite comprises TeO₃²⁻, HTeO₃⁻, H₂TeO₃²⁻, K₂TeO₃, or Na₂TeO₃.

In one example, wherein the concentration of said tellurite is between about 0.4 mM (100 μg/ml) to about 2 mM (500 μg/ml)

In one example, wherein said tellurium nanostructures are formed in the shape of uniform nanorods or and not crystals.

In one example, wherein said tellurium nanostructures are formed in the shape of uniform spherical nanoparticles.

In one example, wherein said tellurium nanostructures that are formed are stable, dispersed and non-aggregated.

In one example, wherein said tellurium nanorods have a length of about 100 nm to about 1000 nm.

In one example, further comprising isolating said produced tellurium nanostructures.

In one example, wherein said isolating comprises collecting said BCP1 cells, washing said collected BCP1 cells, disrupting said collected BCP1 cells, and extracting said tellurium nanostructures from said disrupted BCP1 cells.

In one example, wherein said collecting of said BCP1 cells comprises centrifugation.

In one example, wherein said washing of said collected BCP1 cells comprises washing with a saline solution.

In one example, wherein said disrupting comprises sonication.

In one example, wherein said extracting of said tellurium nanostructures comprises removing the cellular debris following said disrupted cells to obtain a supernatant, and isolating the tellurium nanostructures from said supernatant.

In one aspect there is described a tellurium nanorod produced according to any one of claims 1 to 17.

In one aspect there is described a tellurium nanorod produced according to any one of claims 1 to 17 for use in:

electronics or electronics equipment,

glass or industrial glass,

as alloys, preferably with copper, cadmium or stainless steel,

batteries as an anti-corrosive or semiconductor

ceramic as a colouring agent,

photosensitive semiconductors, optics, quantum dots.

a thin film in solar panels,

in catalysts for petroleum cracking and in blasting caps for explosives,

petroleum refining, or

mining.

antifouling coatings,

antioxidant agents,

human and agricultural pharmaceuticals: antimicrobials, biocides, antifungals, antivirals, anticancer agents,

piezoelectric devices.

In one aspect there is described a method of producing selenium nanostructures, comprising: culturing Rhodococcus aetherivorans (BCP1) bacteria in a medium comprising selenium.

In one example, wherein said culturing comprises pre-culturing said bacteria in said medium to generate a pre-culture, followed by culturing a portion of said pre-culture in said medium comprising selenium to form a first culture.

In one example, further comprising a culturing a portion of said first culture in said medium comprising selenium to form a second culture.

In one example, wherein said culturing is performed under aaerobic conditions.

In one example, wherein said culturing is performed under aerobic conditions at about 20-40° C.

In one example, wherein said selenium comprises SeO₃²⁻, HSeO₃⁻, H₂SeO₃²⁻, K₂SeO₃, Na₂SeO₃, or Na₂SeO₄.

In one example, wherein the concentration of said selenium is between about 0.5 mM to >200 mM , preferably 0.5 mM to 200 mM.

In one example, wherein said selenium nanostructures are formed in the shape of uniform spherical nanoparticles or nanorods and not crystals.

In one example, wherein said selenium nanostructures that are formed are stable, dispersed and non-aggregated.

In one example, wherein said selenium nanoparticles have a diameter of about 50 nm to about 250 nm.

In one example, wherein said nanorods have a length of about 20 nm to about 1000 nm.

In one example, further comprising isolating said produced selenium nanostructures.

In one example, wherein said isolating comprises collecting said BCP1 cells, washing said collected BCP1 cells, disrupting said collected cell, and extracting said selenium nanostructures from said washed BCP1 cells.

In one example, wherein said collecting of said BCP1 cells comprises centrifugation.

In one example, wherein said washing of said collected BCP1 cells comprises washing with a saline solution.

In one example, wherein said extracting of said selenium nanostructures comprises removing the cellular debris following said disrupted cells to obtain a supernatant, and isolating the selenium nanostructures from said supernatant.

In one aspect there is described a selenium nanorod or nanoparticle produced according to any one of claims 20 to 35.

In one aspect there is described a selenium nanorod or nanoparticle produced according to any one of claims 20 to 36 for use in:

electronics or electronics equipment,

glass or industrial glass,

animal feed,

food supplements,

as alloys, preferable an alloy for batteries

production of pigments, or

production of plastics.

optics

production of medical devices.

antifouling coatings,

antioxidant agents,

human and agricultural pharmaceuticals: antimicrobials, biocides, antifungals, antivirals, anticancer agents,

quantum dots.

In one aspect there is described a nanorod produced according to the method of any one of claims 1 to 37, wherein said nanorod is a nanoribbon (flat structure), nanotube (hollow structure) or solid nanorod.

In one aspect there is described an electronic device comprising: a substrate and one or more tellurium nanorods forming an electrically conductive path in said substrate.

In one example, wherein said one or more tellurium nanorods are made according to the method of any one of 1 to 17.

In one aspect there is described an electrically conductive material comprising: a substrate and one or more tellurium nanorods forming an electrically conductive path in said substrate.

In one example, wherein said one or more tellurium nanorods are made according to the method of any one of claims 1 to 17.

In one aspect there is described an electric device comprising an electrically conductive material of claim 41 or 42, wherein said electronic device is a resistor, capacitor, support, semiconductor, or wire.

In one aspect there is described an electronic device comprising: a substrate and one or more selenium nanorods forming an electrically conductive path in said substrate.

In one example, wherein said one or more selenium nanorods are made according to any one of claims 20 to 35.

In one aspect there is described an electrically conductive material comprising: a substrate and one or more selenium nanorods forming an electrically conductive path in said substrate.

In one example, wherein said one or more selenium nanorods are made according to any one of claims 20 to 35.

In one aspect there is described an electric device comprising an electrically conductive material of claim 46 or 47, wherein said electronic device is a resistor, capacitor, support, semiconductor, or wire.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 is a Kill curve of Rhodococcus aetherivorans BCP1 exposed for 24 h to increasing concentration of K₂TeO₃, with the established Minimal Inhibitory Concentration (MIC).

FIG. 2 Rhodococcus aetherivorans BCP1 growth in LB medium, LB supplied with 100 or 500 μg/mL of K₂TeO₃ as unconditioned (a and c) or conditioned (b and d) cells, and TeO₃²⁻consumption.

FIG. 3 Transmission Electron Microscopy (TEM) micrographs of BCP1 cells grown for 120 h in the presence of 100 μg/mL (a), and 500 μg/mL (b) of K₂TeO₃. Arrows indicate the intracellular TeNRs produced by the BCP1 strain.

FIG. 4 Dynamic Light Scattering (DLS) analysis of TeNRs₁₀₀ (a and b), and TeNRs₅₀₀ (c and d) extracted from the BCP1 strain grown as unconditioned (a and c) or conditioned (b and d) cells in the presence of K₂TeO₃.

FIG. 5 depicts Dynamic Light Scattering (DLS) analysis of supernatants recovered from TeNRs₁₀₀ (a and b), and TeNRs₅₀₀ (c and d) extracted from the BCP1 strain grown as unconditioned (a and c) or conditioned (b and d) cells in the presence of K₂TeO₃.

FIG. 6 depicts Transmission Electron Microscopy (TEM) micrographs of TeNRs₁₀₀ (a), and TeNRs₅₀₀ (b) extracted from the BCP1 strain grown as unconditioned cells in the presence of K₂TeO₃, and TeNRs₁₀₀ (c), and TeNRs₅₀₀ (d) recovered from those conditioned.

FIG. 7 depicts Length distribution (nm) of TeNRs₁₀₀ (a), and TeNRs₅₀₀ (b) generated by unconditioned BCP1 K₂TeO₃-grown cells, and TeNRs₁₀₀ (c), and TeNRs₅₀₀ (d) isolated from conditioned ones. Length distributions are indicated as grey filled circles, while the Gaussian fit is highlighted as a continuous black curve.

FIG. 8 depicts Zeta Potential measurements of TeNRs₁₀₀ (a), and TeNRs₅₀₀ (b) generated by unconditioned BCP1 cells, and TeNRs₁₀₀ (c), and TeNRs₅₀₀ (d) extracted from conditioned BCP1 cells grown in the presence of K₂TeO₃.

FIG. 9 depicts Zeta Potential measurements of the supernatants recovered from TeNRs₁₀₀ (a), and TeNRs₅₀₀ (b) generated by unconditioned BCP1 cells, and those of TeNRs₁₀₀ (c), and TeNRs₅₀₀ (d) extracted from conditioned BCP1 cells grown in the presence of K₂TeO₃.

FIG. 10 depicts Scanning Electron Microscopy (SEM) micrographs of TeNRs₁₀₀ (a), and TeNRs₅₀₀ (b) produced by unconditioned BCP1 K₂TeO₃-grown cells, and TeNRs₁₀₀ (c), and TeNRs₅₀₀ (d) extracted from those conditioned.

FIG. 11 depicts Energy-Dispersed X-Ray Spectroscopy (EDX) spectra of TeNRs100 (a), and TeNRs500 (b) unconditioned BCP1 grown cells, and TeNRs100 (c), and TeNRs500 (d) extracted from those conditioned ones grown in the presence of K₂TeO3.

FIG. 12: Tolerance of Rhodococcus aetherivorans BCP1 exposed for 24 h to increasing concentration of Na2SeO3. The Minimal Inhibitory Concentration of SeO32-(MIC^{Se}) was >200 mM.

FIG. 13: Rhodococcus aetherivorans BCP1 growth in LB medium (orange curves), LB supplied with 0.5 or 2 mM of Na₂SeO₃ (black curves) as unconditioned (a and c) or conditioned (b and d) cells, and SeO₃²⁻ consumption indicated by dashed red curves.

FIG. 14: Transmission Electron Microscopy (TEM) micrographs of BCP1 cells grown for 120 h in the presence of 0.5 mM (a), and 2 mM (b) of Na₂SeO₃. Arrows indicate selenium nanostructures (SeNPs and/or SeNRs) produced by the BCP1 strain.

FIG. 15: Transmission Electron Microscopy (TEM) micrographs of unconditioned and/or conditioned generated SeNPs/SeNRs_{0.5} (a and b) and SeNPs/SeNRs₂ (c and d).

FIG. 16: Size distributions (nm) of SeNPs_{0.5} (a), and SeNPs₂ (b) generated by unconditioned BCP1 Na₂SeO₃-grown cells, and SeNPs_{0.5} (c), and SeNPs₂ (d) isolated from the conditioned ones. Size distributions are indicated as red filled circles, while the Gaussian fit is highlighted as a continuous black curve.

FIG. 17: Length distribution (nm) of SeNRs_{0.5} (a), and SeNRs₂ (b) generated by unconditioned BCP1 Na₂SeO₃-grown cells, and SeNRs_{0.5} (c), and SeNRs₂ (d) isolated from those conditioned. Length distributions are indicated as red filled circles, while the Gaussian fit is highlighted as a continuous black curve.

FIG. 18: Scanning Electron Microscopy (SEM) micrographs of SeNPs/SeNRs_{0.5} (a), and SeNPs/SeNRs₂ (b) produced by unconditioned BCP1 Na₂SeO₃-grown cells, and SeNPs/SeNRs_{0.5} (c), and SeNPs/SeNRs₂ (d) extracted from those conditioned.

FIG. 19: Energy-Dispersed X-Ray Spectroscopy (EDX) spectra of SeNPs0.5 (a), SeNPs₂ (b), SeNRs_{0.5} (e) and SeNRs₂ (f) generated by unconditioned BCP1 cells, and SeNPs_{0.5} (c), SeNPs (d), SeNRs_{0.5} (g) and SeNRs₂ (h) extracted from those conditioned ones.

FIG. 20: Dynamic Light Scattering (DLS) plots of selenium nanostructures extracted from BCP1 grown as unconditioned or conditioned cells in the presence of 0.5 mM (a and c; black peaks) or 2 mM (a and c grey peaks) of SeO₃²⁻, as well as for the supernatants recovered after removing the nanomaterials produced by using 0.5 mM (b and d; red peaks) or 2 mM (b and d; blue peaks) of precursor (Na₂SeO₃).

FIG. 21 Zeta Potential measurements of selenium nanostructures generated by unconditioned and conditioned BCP1 cells grown in the presence of 0.5 mM (a and c) or 2 mM (b and d) of Na₂SeO₃.

FIG. 22 Zeta Potential measurements of the supernatants containing selenium nanostructures, generated by unconditioned and conditioned BCP1 cells grown in the presence of 0.5 mM (a and c) or 2 mM (b and d) of SeO₃²⁻ oxyanions.

FIG. 23. (a) Rhodococcus aetherivorans BCP1 resting cells survival curve upon increased initial concentration of TeO₃²⁻, being 100 ( [symbol] ), 500 ( [symbol] ) or 1000 ( [symbol] ) μg mL⁻¹, while in (b) is shown the initial depletion rate (O) of TeO₃²⁻. The linear correlation ( [symbol] ) that fits the experimental data points gave an R²=0.97. In (c) is reported the percentage of TeO₃²⁻ removal over the considered timeframe for each initial oxyanion concentration [100 (▪), 500 ( [symbol] ) or 1000 (▪) μg mL⁻¹]. The error bars indicate the standard deviation of three biological replicates.

FIG. 24. Transmission Electron Microscopy observations of Rhodococcus aetherivorans BCP1 resting cells exposed to different concentrations (100, 500 and 1000 μg mL⁻¹) of TeO₃²⁻ either for 0.5 (a, c and e) or 16 h (b, d and f); TeNPs and TeNRs within the cells are indicated by black arrows.

FIG. 25. Transmission Electron micrographs of Te-nanostructure extracts recovered from Rhodococcus aetherivorans BCP1 resting cells after either 0.5 (a, c and e) or 16 h (b, d and f) exposure to 100, 500 and 1000 μg mL-1 of TeO₃²⁻; spherical and rod-shaped Te-nanostructures, as well as shard-like NPs are indicated by black and white arrows, respectively.

FIG. 26. Length distribution ( [symbol] ) of TeNRs generated by Rhodococcus aetherivorans BCP1 resting cells exposed for either 1 or 16 h to 100, 500 and 1000 μg mL⁻¹ of TeO₃²⁻. The Gaussian fit is indicated by ( [symbol] ).

FIG. 27. (a) Exponential trend of growth of TeNRs average length as function of time, when the BCP1 strain is exposed to 100 (●), 500 ( [symbol] ) or 1000 (▴) μg mL-1 of TeO₃²⁻. In (b) is reported the linear correlation of the TeNRs average length measured as function of the initial TeO₃²⁻ precursor per each time point [1 (♦), 3 (▪), 6 (▴) and 16 h (●)] of BCP1 resting cells exposure, with R² values of 0.99, exception made for the 3 h time point, which resulted to be 0.94. The error bars represent the standard deviation derived from the measurements of 100 randomly chosen TeNRs.

FIG. 28. (a) Bright-field electron micrograph of a single TeNR; (b) High-Resolution 538 micrograph that highlights the [010] growth plane of TeNR crystal. The enlarged insert (b 1) displays the interplanar distance of the periodic fringe spacing, while (c) shows the corresponding electron diffraction pattern in which the diffraction spots [101] and are indexed.

FIG. 29 depicts abiotic control experiments. Evaluation of TeO₃²⁻ removal when it was supplied to PBS ( ) or PBS containing autoclaved biomass ( ) over the incubation time. The error bars indicate the standard deviation three biological replicates.

FIG. 30 depicts Transmission Electron Microscopy imaging of BCP1 resting cells exposed to 100 μg mL⁻¹ of TeO₃²⁻. Intracellular formation of Te-nanostructures over time. The biogenic Te-nanomaterial in the form of Te-nanoparticles (TeNPs) and Te-nanorods (TeNRs) is highlighted by black arrows. Scale bar=100 nm.

FIG. 31 depicts Transmission Electron Microscopy imaging of BCP1 resting cells exposed to 500 μg mL⁻¹ of TeO₃²⁻. Intracellular formation of Te-nanostructures over time. The biogenic Te-nanomaterial in the form of Te-nanoparticles (TeNPs) and Te-nanorods (TeNRs) is highlighted by black arrows. Scale bar=500 nm.

FIG. 32 depicts Transmission Electron Microscopy imaging of BCP1 resting cells exposed to 1000 μg mL⁻¹ of TeO₃²⁻. Intracellular formation of Te-nanostructures over time. The biogenic Te-nanomaterial in the form of Te-nanoparticles (TeNPs) and Te-nanorods (TeNRs) is highlighted by black arrows. Scale bar=500 nm.

FIG. 33 depicts Transmission Electron Microscopy imaging of Te-nanostructure extracts generated by BCP1 resting cells exposed to 100 μg mL⁻¹ of TeO₃²⁻. Electron micrographs of the biogenic Te-nanomaterial recovered from BCP1 cells over the exposure time. Te-nanomaterial in the form of Te-nanoparticles (TeNPs) and Te-nanorods (TeNRs) is highlighted by black arrows. Scale bar=100 nm.

FIG. 34 depicts Transmission Electron Microscopy imaging of Te-nanostructure extracts generated by BCP1 resting cells exposed to 500 μg mL-1 of TeO₃²⁻. Electron micrographs of the biogenic Te-nanomaterial recovered from BCP1 cells over the exposure time. Te-nanomaterial in the form of Te-nanoparticles (TeNPs) and Te-nanorods (TeNRs) is highlighted by black arrows. Scale bar=100 nm.

FIG. 35 depicts Transmission Electron Microscopy imaging of Te-nanostructure extracts generated by BCP1 resting cells exposed to 1000 μg mL⁻¹ of TeO₃²⁻. Electron micrographs of the biogenic Te-nanomaterial recovered from BCP1 cells over the exposure time. Te-nanomaterial in the form of Te-nanoparticles (TeNPs) and Te-nanorods (TeNRs) is highlighted by black arrows, while the white arrows indicate the shard-like nanoparticles. Scale bar=100 nm.

FIG. 36 depicts Tellurium nanorods (TeNRs) average length distribution. Dependency of the biogenic TeNRs average length ( [symbol] ) measured on the initial TeO₃²⁻ concentration and cell exposure time. The distribution was fitted to a Gaussian function ( [symbol] ) to yield TeNRs average length.

### DETAILED DESCRIPTION

Generally, in one aspect, the present disclosure provides a method and system for producing tellurium nanostructures.

In one example the present disclosure provides a method of producing tellurium nanostructures in a bacterium.

The TeO₃²⁻-reducing bacteria described herein convert TeO₃²⁻ to the less toxic elemental tellurium (Te⁰), which accumulated intracellularly.

In a specific example the bacterium is a Gram-positive bacterium. In a specific example, the bacterium belongs to the Rhodococcus genus, belonging to the Mycelia group of Actinomycetes. In a specific example, there are aerobic non-sporulating bacteria with a high G+C content.

In a more specific example, the bacterium is Rhodococcus aetherivorans BCP1 (DSM 44980).

Other specific examples are the bactiera Paenibacillus TeW, Salinicoccus sp. QW6, Bacillus beveridgei, Bacillus selenitireducens, or Rhodobacter capsulatus B100.

In a specific example of the methods herein, the bacteria are cultured under anerobic conditions at about 30° C.

In a specific example, the bacteria are cultured in the presence of tellurite (TeO₃²⁻).

The tellurite (TeO₃²⁻) may be obtained from a variety of sources.

For example, Te is normally present in the environment as inorganic telluride (Te²), the oxyanions tellurite (TeO₃²⁻) and tellurate (TeO₄²), and the organic dimethyl telluride (CH₃TeCH₃). TeO₃²⁻ is the most soluble form of tellurium. Due to tellurite's use in electronics as well as industrial glasses, it can be found highly concentrated in soil and water near waste discharge sites of manufacturing and processing facilities, as a hazardous and toxic pollutant.

In some examples, the source of tellurite (TeO₃²⁻) to be used in the production of tellurium nanostructures comprises K₂TeO₃. In a specific example, the concentration of tellurite is between 0.4 mM (100 μg/ml) to 500 mM (500 μg/ml).

In one example, the tellurite (TeO₃²⁻) is added at the concentration of 100 μg/ml to the bacterial culture. In another example, the tellurite (TeO₃²⁻) is added at a concentration five times (500 μg/ml) more compared to the previous one.

In a specific example, the tellurium nanostructures are formed in the shape of nanorods. In some examples the tellurium nanorods have a length of about 125 nm to about 610 nm.

The tellurium nanorods produced may be isolated from the bacteria.

In one example, the tellurium nanorods are isolated from the collected bacterial cells. The cells are washed and disrupted by sonication. The tellurium nanostructures are recovered from the disrupted cells.

The bacterial cells may be collected in a variety of ways, as would be known to the skilled worker. In one example, the cells are collected by centrifugation. In another example, the bacterial cells are collected by filtration.

The bacterial cells may be washed one or more times, using the same or differing washing media. In a specific example, the washing media is a saline solution.

The bacterial cells may be disrupted in a variety of ways, as would be knows to the skilled worker. In a specific example, disrupting comprises sonication. Additional non limiting example of disrupting methods include physical cell lysis by grinding, and/or pressure, and/or chemical cell lysis utilizing solutions of detergents.

Extracting the tellurium nanostructures comprises removing the cellular debris following disruption to obtain a supernatant, and isolating the tellurium nanostructures from said supernatant.

The tellurium nanorods may then be purified from the supernatant.

The purified tellurium nanorods may be used in a variety of industrial applications, including but not limited to, use in: electronics or electronics equipment, glass or industrial glass, as alloys, preferably with copper or stainless steel, batteries as an anti-corrosive ceramic as a coloring agent, photosensitive semiconductors, a thin film in solar panels, in catalysts for petroleum cracking and in blasting caps for explosives, petroleum refining, or mining, antimicrobials, antifungals, antivirals, biocides, antifouling coatings, piezoelectric devices, quantum dots.

In one example, there is described an electronic device comprising a substrate and one or more tellurium nanorods forming an electrically conductive path in said substrate.

In one example, there is described an electrically conductive material comprising: a substrate and one or more tellurium nanorods forming an electrically conductive path in said substrate.

An electric device comprising an electrically conductive material as described above, wherein said electronic device is or comprises a resistor, capacitor, support, semiconductor, or wire.

In some examples, the substrate may include but is not limited to, an inorganic material such as glass, or an organic material such as polycarbonate, olymethylmethacrylate, polyethylene terephthalate, polyethylene naphthalate, polyamide, polyethersulfone, or a combination thereof, a silicon wafer or support, and the like. In one example, the substrate is a silicone support. In one example, the support is a semiconductor.

Method of applying the one or more tellurium nanorods will be known to the skilled worker.

Non limiting examples of devices in which tellurium nanorod may be used include microelectronics of sensors (optical or electronic) which may require solid state, gel or flexible electronics.

In another aspect, the present disclosure provides a method and system for producing selenium nanostructures.

In one example the present disclosure provides a method of producing selenium nanostructures in a bacterium.

The SO₃²⁻-reducing bacteria described herein convert SeO₃²⁻ to the less toxic elemental tellurium (Se⁰), which accumulated on the outer surface of the cells.

In a specific example, the bacterium is a Gram-positive bacterium. In a specific example, the bacterium belongs to the Rhodococcus genus, belonging to the

Mycolata group of Actinomycetes. In a specific example, there are aerobic non-sporulating bacteria with a high G+C content.

In a more specific example, the bacterium is Rhodococcus aetherivorans BCP1 strain (DSM 44980).

Other specific examples are the bactiera are Geobacter sulfurreducens, Shewanella oneidensis, Veillonella atypica, Rhodospirillum rubrum, Sulfurospirillum bamesii, Bacillus selenitireducens or Selenihalanerobacter shrifiti.

In a specific example of the methods herein, the bacteria are cultured under anerobic conditions at about 30° C.

In a specific example, the bacteria are cultured in the presence of selenite (SeO₃²⁻).

The selenite (SeO₃²⁻) may be obtained from a variety of sources.

Se is present in environment source due to anthropogenic activities such as the anode muds produced during the electrolytic refining of copper, the oil refining, and phosphate and metal ore mining. Additionally, and due to its physical-chemical properties (e.g., relatively low melting point, high photo- and semi-conductivity, optical responses and catalytic activity), Se is used in several applications fields: electronic and glass industries, animal feeds and food supplements, metal alloys for batteries, production of pigments and plastics. Considering its broad use, Se is present in the environment in four inorganic forms: Selenate (SeO₄²⁻) and Selenite (SeO₃²⁻) oxyanions, Selenide (Se²⁻), and elemental Selenium (Se0).

In some examples, the source of selenite (SeO₃²⁻) to be used in the production of selenium nanostructures comprises Na₂SeO₃. In a specific example, the concentration of said selenite is between 0.5 mM to 200 mM, preferably about 0.5 mM to about 2 mM.

In one example, the selenite (SeO₃²⁻) is added at a concentration of 0.5 mM to the culture of bacteria. In another example, the selenite (SeO₃²⁻) is added at a concentration 4 times (2 mM) higher than the previous one.

In a specific example, the selenium nanostructures are formed in the shape of nanorods and/or nanoparticles.

In some examples the selenium nanoparticles have a size of about 50 nm to about 149 nm.

In some examples, the selenium nanorods have a length of about 33 nm to about 863 nm.

The selenium nanorods and nanoparticles produced may be isolated from the bacteria.

In one example, the selenium nanorods and nanoparticles are isolated from the collected bacterial cells. The cells are washed and disrupted by sonication. The selenium nanostructures are recovered from the disrupted cells.

The bacterial cells may be collected in a variety of ways, as would know to the skilled worker. In one example, the cells are collected by centrifugation. In another example, the bacterial cells are collected by filtration.

The bacterial cells may be washed one or more times, using the same or differing washing media. In a specific example, the washing media is a saline solution.

The bacterial cells may be disrupted in a variety of ways, as would be knows to the skilled worker. In a specific example, disrupting comprises sonication. Additional non limiting example of disrupting methods include physical cell lysis by grinding, and/or pressure, and/or chemical cell lysis utilizing solutions of detergents.

Extracting the selenium nanorods and nanoparticles comprises removing the cellular debris following disruption to obtain a supernatant, and isolating the selenium nanostructures from said supernatant.

The selenium nanorods and nanoparticles may then be purified from the supernatant.

The purified selenium nanorods and nanoparticles may be used in a variety of industrial applications, including but not limited to use in: electronics or electronics equipment, glass or industrial glass, as alloys, preferable an alloy for batteries, production of pigments, or production of plastics, antimicrobials, biocides, antifungals, antivirals, biocides, antifouling coatings, anticancer agents, optics, antioxidant agents, quantum dots.

In one example, there is described an electronic device comprising a substrate and one or more selenium nanorods forming an electrically conductive path in said substrate.

In one example, there is described an electrically conductive material comprising: a substrate and one or more selenium nanorods forming an electrically conductive path in said substrate.

An electric device comprising an electrically conductive material of claim as described above, wherein said electronic device is a resistor, capacitor, support, semiconductor, or wire.

In some examples, the substrate may include but is not limited to, an inorganic material such as glass, or an organic material such as polycarbonate, olymethylmethacrylate, polyethylene terephthalate, polyethylene naphthalate, polyamide, polyethersulfone, or a combination thereof, a silicon wafer or support, and the like. In one example, the substrate is a silicone support. In one example, the support is a semiconductor.

Method of applying the one or more selenium nanorods will be known to the skilled worker.

Non limiting examples of devices in which selenium nanorod may be used include microelectronics of sensors (optical or electronic) which may require solid state, gel or flexible electronics.

Method of the invention are conveniently practiced by providing the compounds and/or compositions used in such method in the form of a kit. Such kit preferably contains the composition. Such a kit preferably contains instructions for the use thereof.

In one example, the kit comprises Rhodococcus aetherivorans BCP1.

In one example, the kit comprises a source of tellurite (TeO₃²⁻). In a specific example, the kit comprises K₂TeO₃.

In one example, the kit comprises a source of selenite (SeO₃²⁻). In a specific example, the kit comprises Na₂SeO₃.

To gain a better understanding of the invention described herein, the following examples are set forth. It should be understood that these examples are for illustrative purposes only. Therefore, they should not limit the scope of this invention in anyways.

### EXAMPLES

### Example I

Tellurium (Te) was discovered by Franz-Joseph Müller von Reicheinstein in 1782 [1], and in nature this element can be found in gold ores as association with metals, forming calaverite (AuTe₂), sylvanite (AgAuTe₄) and nagyagite [AuPb(Sb, Bi)Te₂₋₃S6] [2]. Te is an element of the chalcogen family, belonging to the Group 16 of the periodic table along with oxygen (O, sulfur (S), selenium (Se), and the radioactive element polonium (Po) [3].

Additionally, it is defined as a metalloid due to its intermediate properties between metals and non-metals [3]. Due to the anthropogenic activity, Te is normally present in the environment as inorganic telluride (Te²), the oxyanions tellurite (TeO₃²⁻) and tellurate (TeO₄²⁻), and the organic dimethyl telluride (CH₃TeCH₃) [4]. Among these, TeO₃²⁻ is the most soluble form of tellurium, and it is the most toxic form for both prokaryotes and eukaryotes [5] at concentrations as low as 1 μg/mL [6]. This concentration is several orders of magnitude lower as compared to others metals and metalloids of public health and environmental concern such as selenium, iron, mercury, cadmium, copper, chromium, zinc, and cobalt [7,8]. Furthermore, due to tellurite's use in electronics as well as industrial glasses, it can be found highly concentrated in soil and water near waste discharge sites of manufacturing and processing facilities [9], as a hazardous and toxic pollutant [6]. Despite TeO₃²⁻ toxicity, several Gram-negative microorganisms capable to grow phototrophycally or chemotrophycally under aerobic and anaerobic conditions have been described for their capability to reduce this toxic oxyanion, such as Rhodobacter capsulatus B100, Shewanella odeinensis MR-1, Pseudomonas pseudoalcaligenes KF707, and Escherichia coli HB101 strain [10,11,12,13]. Additionally, α-Proteobacteria resistant to concentrations of TeO₃²⁻ ranging from 1 to 25 mg/mL [14,15] and a few Gram-positive strains (e.g., Bacillus beveridgei sp.nov., Bacillus selenitireducens, Corynebacterium diphtheria, Lysinibaci/lus sp. ZYM-1, Bacillus sp. BZ, Bacillus sp. STG-83, Paenibacillus TeW, and Salinicoccus sp. QW6) resistant to low level of TeO3 2-(ranging from 0.2 to 3 mg/mL) were also reported [16,17,18,19,20,21,22,23].

It has been established that TeO₃²⁻-reducing bacteria are able to convert this oxyanion to the less toxic elemental tellurium (Te⁰), which is cytosolically accumulated as black inclusions [6] and/or defined nanostructures such as nanocrystals, nanorods (NRs) and nanoparticles (NPs) [24]. Particularly, Kim and colleagues [25] showed the capability of Shewanella oneidensis MR-1 to produce tellurium nanorods (TeNRs), while Rhodobacter capsulatus B100 is able to produce both intra- and extra-cellular needle-shaped Te-nanocrystals [10]. Another example is the synthesis of tellurium nanoparticles (TeNPs) in cells of Ochrobactrum MPV-1 [26].

NPs and NRs have different physical-chemical and biological properties compared to their bulk counterparts, due to their size, high surface-volume ratio, large surface energy and spatial confinement, allowing the use of these nanostructures in biomedical, electronic, environmental, and renewable energy fields, to name a few [24]. In this context, the natural ability of microorganisms to generate nanostructures by the reduction of toxic oxyanions can play two key roles: (i) the development of eco-friendly “green-synthesis” methods for the production of NPs or NRs [27], and (ii) the decontamination of metal polluted environments [28]. Moreover, the biological synthesis of either NPs or NRs has several advantages over the chemical one, namely: (i) it does not require the use of toxic chemicals; (ii) it does not result in the formation of hazardous wastes; and (iii) it has a substantial lower cost of production [29].

Strains of the Rhodococcus genus, belonging to the Mycolata group of Actinomycetes, are aerobic non-sporulating bacteria with a high G+C content. They are ideal microorganisms for bioremediation and industrial uses due to their remarkable capacity to catalyze a very wide range of compounds and their environmental robustness [30]. Although the ability of Rhodococcus spp. to degrade xenobiotics along with their physiological adaptation strategies, i.e. cell membrane composition and intracellular inclusions, were largely reported in the literature [31], much less is known about the Rhodococcus genus capacity to resist to toxic metals/metalloids. In this respect, Rhodococcus aetherivorans BCP1, a hydrocarbon- and chlorinated solvent degrader that was recently described for its unique capacity to overcome stress environmental conditions in the presence of a wide range of antimicrobials and toxic metals/metalloids such as tellurite, arsenate and selenite [32,33,34,35,36] appears to be an interesting candidate to study. Thus, the present work investigates the ability of Rhodococcus aetherivorans BCP1 to survive in the presence of increasing concentrations of tellurite and to produce Te-nanostructures. In particular, we evaluated the capacity of BCP1 strain to grow in the presence of high concentrations of TeO₃²⁻ oxyanions supplied as K₂TeO₃. TeO₃²⁻ consumption rates were also assessed after re-inoculation of pre-exposed cells in fresh medium with new addition of K₂TeO³ (conditioned cells). Finally, the production of Te-nanostructures was investigated through the use of physical-chemical methods.

### Materials and Methods

Bacterial Strain, Growth Media, Culture Conditions

The strain Rhodococcus aetherivorans BCP1 (DSM 44980) was pre-cultured in 250 mL Erlenmeyer Baffled Flask for 2 days, containing 25 mL of Luria-Bertani medium (here indicated as LB) [composed of (g/L) NaCl, 10; Yeast Extract, 5; Tryptone, 10]. When necessary, the medium was solidified by adding 15 g/L of Agar. BCP1 cells were then inoculated (1% v/v) and grown for 5 days in 50 mL of LB medium supplied with either 100 (0.4 mM) or 500 (2 mM) μg/mL of K₂TeO₃. Here we refer to this first bacterial growth as unconditioned. After this growth step, BCP1 cells were re-inoculated (1% v/v) and cultured for other 5 days in 50 mL of fresh LB medium and 100 or 500 μg/mL of K₂TeO₃. This secondary bacterial growth is here defined as conditioned. Each culture was incubated aerobically at 30° C. with shaking (150 rpm). In order to evaluate the bacterial growth rate, every 24 h an aliquot (100 μL) of BCP1 cells was collected from each culture and serially diluted in sterile saline solution (NaCl 0.9% w/v). The cells were recovered on LB agar plates for 48 h at 30° C. The number of growing cells is reported as average of the Colony Forming Unit per milliliter (CFU mL⁻¹) counted for each biological trial (n=3) with standard deviation. All the reagents were purchased from Sigma-Aldrich®.

Evaluation of TeO₃²⁻ Minimal Inhibitory Concentration (MIC)

In order to establish the Minimal Inhibitory Concentration (MIC) of tellurite, i.e. as the concentration of K₂TeO₃ at which no bacterial growth was observed, the BCP1 strain was exposed to concentrations of K₂TeO₃ ranging from 100 to 3000 μg/mL (0.4 to 12 mM). After 24 h of incubation the number of viable cells was determined by spot plates count on LB agar recovery plates. The assay was conducted in triplicate and the data are reported as average of the CFU mL-1 counted with standard deviation. The established MIC and corresponding kill curve was used to choose the best concentration of K₂TeO₃ to use for nano-material production.

TeO₃²⁻ Consumption Assay

The residual concentration of TeO₃²⁻ oxyanions in the culture broth was estimated as described elsewhere [37]. Briefly, 1 mL of BCP1 cells grown as unconditioned or conditioned in the presence of K₂TeO₃ was collected every 12 h up to 120 h. The sample was centrifuged at 14,000 rpm for 2 min in order to separate the bacterial cell pellet from the supernatant, and a 10- to 100 μL aliquot was mixed with 600 μL of 0.5 M Tris-HCl buffer pH 7.0 (VWR®), 200 μL of diethyldithiocarbamate (Sigma-Aldrich®), and LB up to a total volume of 1 mL. The absorbance of the mixture was read at 340 nm using a Varian Cary® 50 Bio UV-Visible Spectrophotometer. The residual concentration of TeO₃²⁻ oxyanions was determined using this absorbance values and the calibration curve obtained for known concentrations (0, 10, 20, 30, 40, 50 and 60 μg/mL) of K₂TeO₃ in LB (R2=0.99). The data are reported as average values (n=3) with standard deviation.

Preparation, Extraction, and Purification of TeNRs

In order to extract and purify TeNRs produced by the BCP1 strain grown as unconditioned or conditioned cells, biomasses were collected by centrifugation (3700 rpm) for 20 min after 5 culturing days. The pellets were washed twice with saline solution (NaCl 0.9% w/v) and resuspended in Tris-HCl (1.5 mM) buffer pH 7.4. Bacterial cells were disrupted by ultrasonication at 22 W for 10 min (30 seconds burst interspersed by 30 seconds of pause) on ice (MICROSON™ Ultrasonic Cell Disruptor XL, Qsonica Misonix Inc.). The cellular debris was then separated from TeNRs in the supernatant by a centrifugation step (3700 rpm) for 20 min. Supernatants containing TeNRs were incubated overnight (16 h) at 4° C. with 1-Octanol (Sigma-Aldrich®) in a ratio 4:1 (v/v) and then recovered by centrifugation (16,000 rpm) for 15 minutes. TeNRs pellets were finally suspended in deionized water.

Here we refer to the TeNRs produced by the BCP1 strain as TeNRs₁₀₀ or TeNRs₅₀₀, depending on the initial concentration of K₂TeO₃ present in the growth medium.

Dynamic Light Scattering (DLS) and Zeta Potential Measurements

DLS and Zeta potential measurements of TeNRs produced by BCP1 cells grown as unconditioned or conditioned were performed using a Zen 3600 Zetasizer Nano ZSTM from Malvern Instruments. The samples (1 mL each) were analyzed in a spectrophotometric cuvette (10×10×45 mm Acrylic Cuvettes, Sarstedt) and in a folded capillary Zeta cell (Malvern Instruments) for DLS and Zeta potential measurements, respectively.

Transmission Electron Microscopy (TEM) Analysis

TEM observations of TeNRs extracted from BCP1 cells grown as unconditioned or conditioned were carried out by mounting 5 μL of each sample on carbon-coated copper grids (CF300-CU, Electron Microscopy Sciences), air-drying the samples, and imaging them using a Hitachi H7650 TEM. The distribution of TeNRs length was calculated by measuring the length of 100 randomly chosen nanorods through the use of ImageJ software. The distribution was fitted to a Gaussian function to yield the average length. In order to image BCP1 cells grown in the presence of 100 or 500 μg/mL K₂TeO₃ for 5 days, the cells were negatively stained using a 1% phosphotungstic acid solution (pH 7.3).

Scanning Electron Microscopy (SEM) and Energy-Dispersed X-ray Spectroscopy (EDX) Analysis

The samples were prepared by depositing TeNRs suspensions onto Crystal Silicon wafers (type N/Phos, size 100 mm, University Wafer) and air-drying. Imaging and EDX analysis were performed on a Zeiss Sigma VP scanning electron microscope and an Oxford Instruments INCAx-act system, respectively.

### Results

Minimal Inhibitory Concentration (MIC) assay of Rhodococcus sp. BCP1 Strain

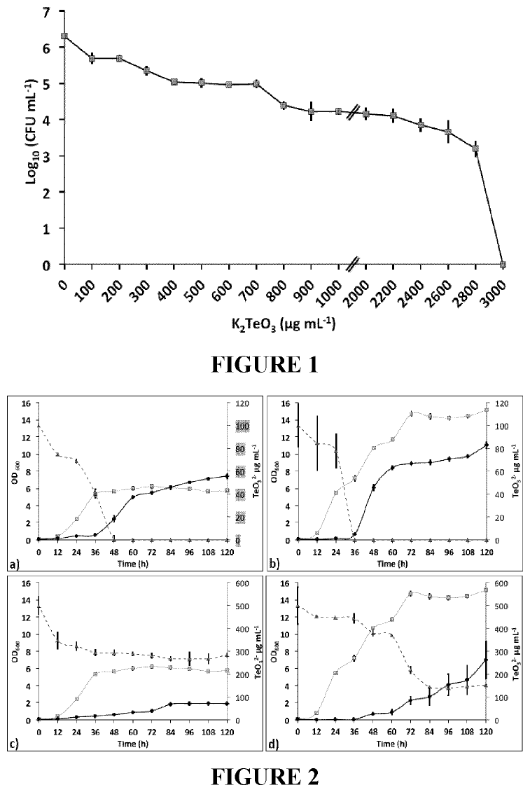

In order to evaluate the BCP1 strain's ability to tolerate TeO₃²⁻ oxyanions present in the growth medium (LB), the MIC was established by exposing the cells for 24 h to different K₂TeO₃ concentrations, ranging from 0 to 3000 μg/mL (0-12 mM). The data are plotted in FIG. 1 as a kill curve displaying the number of BCP1 viable cells against the K₂TeO₃ concentration values. As a result, the MIC value of TeO₃²⁻ was estimated at 2800 μg/mL (11.2 mM) that corresponded to 3 log reduction as compared to the number of viable cells counted at the time of inoculation, while only 1 and 2 log reduction of BCP1 viable cells was observed when the K₂TeO₃ was varied from 100 to 1000 μg/mL (0.4-4 mM) and from 100 to 2000 μg/mL (0.4-8 mM), respectively.

Growth and Consumption of TeO32- by the BCP1 Strain, and Localization of TeNRs

Since the number of BCP1 viable cells decreased by less than 1 log after 24 h exposure to 100 μg/mL (5.00·10⁵ CFU/mL) or 500 μg/mL (1.00·10⁵ CFU/mL) of K₂TeO₃, the growth and consumption of TeO₃²⁻ at these concentrations by the BCP1 strain were evaluated for both unconditioned and conditioned grown cells (FIG. 2). Unconditioned BCP1 cells grown in the presence of 100 μg/mL of K₂TeO₃ showed an initial consumption of the oxyanions during their lag phase (24 h), while a complete reduction occurred in the early exponential growth phase (48 h), showing a stationary phase after 60 h of growth (FIG. 2a). In the case of conditioned BCP1 cells the reduction of the same amount of TeO₃²⁻ was 12 h faster (36 h) as compared to those grown as unconditioned, occurring in the early exponential growth phase. As for unconditioned cells, the conditioned ones reached the stationary phase after 60 h of incubation and any lag phase of growth was observed (FIG. 2b). By contrast, considering unconditioned BCP1 cells growing in the presence of 500 μg/mL of K₂TeO₃, the consumption/reduction of the oxyanions was not complete over the incubation time (120 h), resulting in the reduction of about 45% (218 μg) of the initial amount of TeO₃²⁻ (FIG. 2c). Particularly, the initial amount of the oxyanions decreased by 153 μg during the lag phase of growth (24 h), reaching the maximum extent of reduction after 72 h of incubation (282 μg), and it remained constant over the stationary growth phases (FIG. 2c). Regarding conditioned BCP1 K₂TeO₃-grown cells in the presence of 500 μg/mL, we did not observe a complete reduction of the initial TeO₃²⁻ concentration, although the amount of residual oxyanions present in the medium was lower (152 μg) as compared to unconditioned grown cells. Specifically, a reduction of 56 μg of TeO₃²⁻ oxyanions during the initial 36 h of incubation was observed, which corresponds to the lag phase of growth, while after 84 h TeO₃²⁻ oxyanions concentration dropped down to its minimal value, along with an actual growth of the biomass (FIG. 2d).

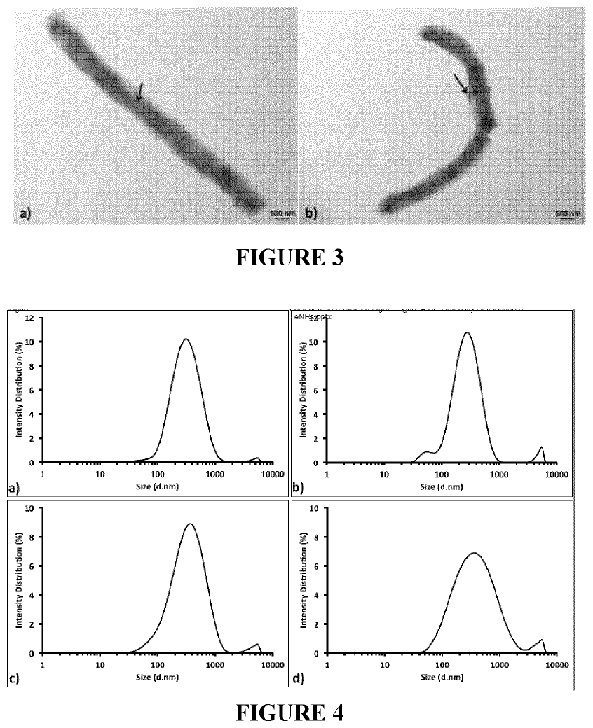

To detect the production of tellurium nanostructures by BCP1, either 100 or 500 μg/mL K₂TeO₃-grown cells for 5 days were negatively stained and analyzed by TEM (FIG. 3). In both cases, the presence of intracellular TeNRs was detected (FIG. 3a and b).

Dynamic Light Scattering (DLS) Analyses

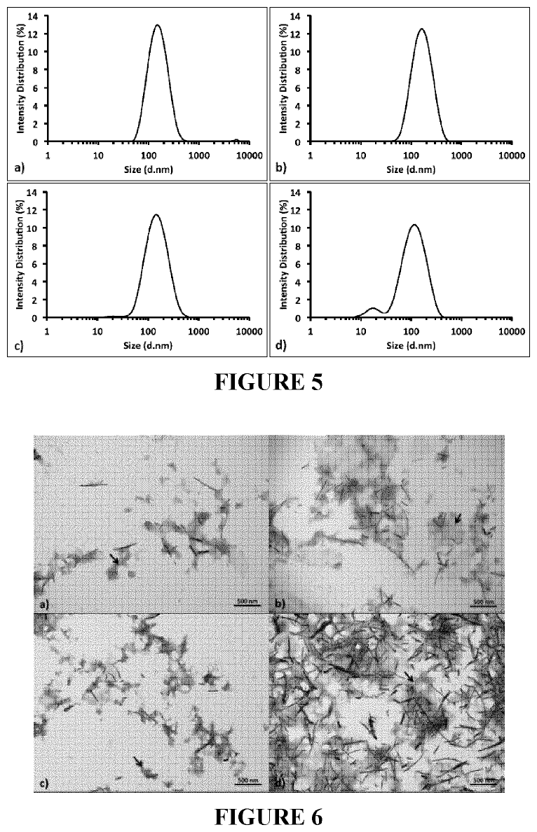

DLS experiments were performed on TeNRs extracted from BCP1 unconditioned and conditioned grown cells (FIG. 4). The measurements yielded distributions of sizes centered at 295 nm (FIG. 4a and b) for the samples of TeNRs₁₀₀ produced by BCP1 strain grown as unconditioned or conditioned cells, with a standard deviation of ±61 nm (unconditioned) and ±22 nm (conditioned). TeNRs₅₀₀ isolated from unconditioned and conditioned grown cells were featured by a size distribution centered at 342 nm (FIG. 4c and d), with a standard deviation of ±64 nm and ±86 nm, respectively. The TeNRs populations were found to be polydisperse as indicated by the values of the measured polydispersity index, being 0.398 (TeNRs₁₀₀) and 0.395 (TeNRs₅₀₀) for Te-nanostructures generated by unconditioned BCP1 cells, and 0.384 (TeNRs₁₀₀) and 0.381 (TeNRs₅₀₀) for those isolated from conditioned cells. Additional DLS experiments were performed on the supernatants containing TeNRs, which were recovered by removing TeNRs from the samples through centrifugation at 8000 rpm for 10 minutes. The DLS measurements performed on the supernatants (FIG. 5) produced distributions shifted towards smaller sizes compared to the ones obtained from the samples containing the nanorods (FIG. 4): 142±14 nm and 164±9 nm (FIG. 5a and b) for the supernatants recovered after removing TeNRs₁₀₀ produced by BCP1 grown as unconditioned or conditioned cells, and 142±17 nm and 122±12 nm (FIG. 5c and d) for the supernatants obtained after removing TeN RS₅₀₀ generated by the cells grown as unconditioned or conditioned, respectively. As a control, DLS analysis of the supernatant derived from the BCP1 culture grown for 120 h on rich medium (LB) showed a peak centered at 1±0.48 nm (FIG. 5e), which is likely due to the presence of peptides in the culture broth.

Transmission Electron Microscopy (TEM) Analysis and Size Distribution of TeNRs

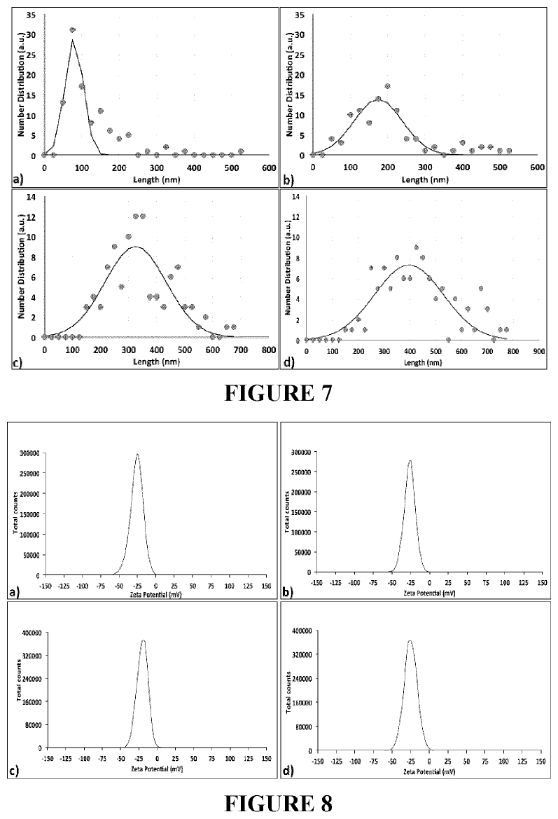

TEM observations were carried out on extracted TeNRs in order to study the size and morphology of TeNRs produced by both unconditioned and conditioned cells (FIG. 6). TeNRs from unconditioned cells revealed the presence of electron-dense and not aggregated NRs showing variability in length (FIG. 6a and b). Particularly, the length measurements using ImageJ software of 100 randomly chosen NRs yielded an average size of 148±104 nm and 223±116 nm for TeNRs₁₀₀ and TeN RS₅₀₀, respectively (FIG. 6a and b). High electron-density was observed in TeNRs extracted from conditioned cells as well (FIG. 6c and d). TeNRs₁₀₀ or TeNRs₅₀₀ isolated from BCP1 conditioned cells were longer compared to those generated by unconditioned cells, with a broader length distribution. In this case, the evaluated average size of NRs is 354±125 nm and 463±147 nm for TeNRs₁₀₀ and TeNRs₅₀₀, respectively (FIG. 7c and d). Furthermore, the TEM analyses of TeNRs extracted from either unconditioned or conditioned cells revealed the presence of an electron-dense material surrounding the nanorods (FIG. 6, indicated by arrows).

Zeta Potential Measurement

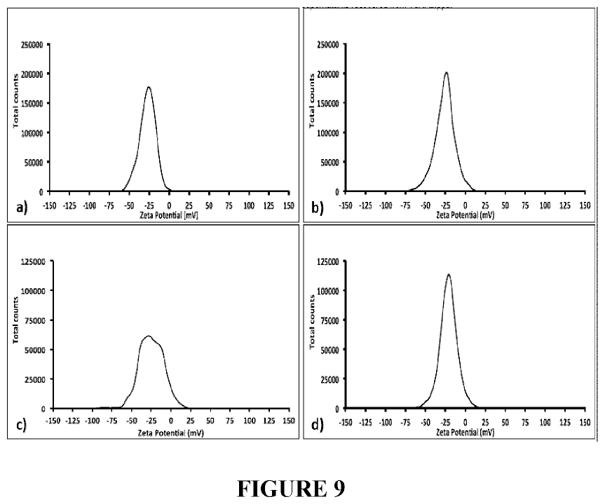

Zeta potential measurements were conducted to evaluate whether the surface of TeNRs was charged (FIG. 8). A single peak at -25 mV was detected in Zeta potential plots for both unconditioned generated TeNRs₁₀₀ and TeN RS₅₀₀ (FIG. 8a and b). The Zeta potential results obtained for TeNRs produced by conditioned BCP1 cells indicated the presence of a less negative potential (−20 mV) in the case of TeNRs₁₀₀, while TeN RS₅₀₀ were featured by the same potential value of unconditioned NRs (−25 mV) (FIG. 8c and d). Similarly to the DLS analysis, additional Zeta potential measurements were performed on the supernatants recovered after removing TeNRs through centrifugation (FIG. 9), resulting in similar surface potential values as compared to those obtained for TeNRs suspensions.

Particularly, the supernatants recovered from TeNRs produced by unconditioned cells grown in the presence of either 100 or 500 μg/mL of K₂TeO₃ were featured by a surface potential of −26 and −22 mV (FIG. 9a and b), while those obtained from TeNRs₁₀₀ and TeNRs₅₀₀ generated by conditioned cells had a charge of −29 and −21 mV (FIG. 9c and d), respectively.

Scanning Electron Microscopy (SEM) and Energy-Dispersed X-Ray Spectroscopy (EDX) Analyses

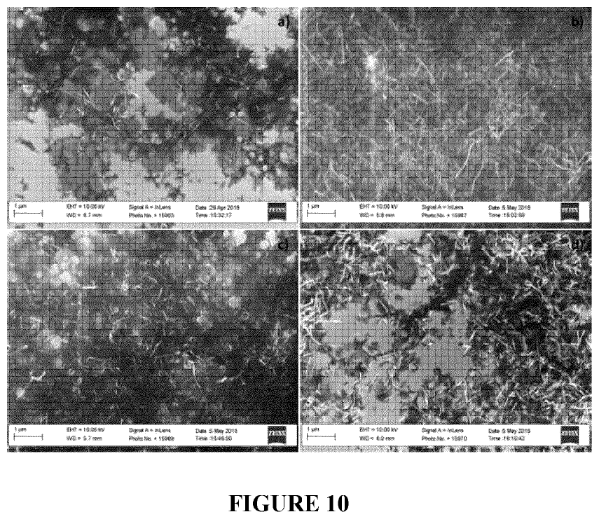

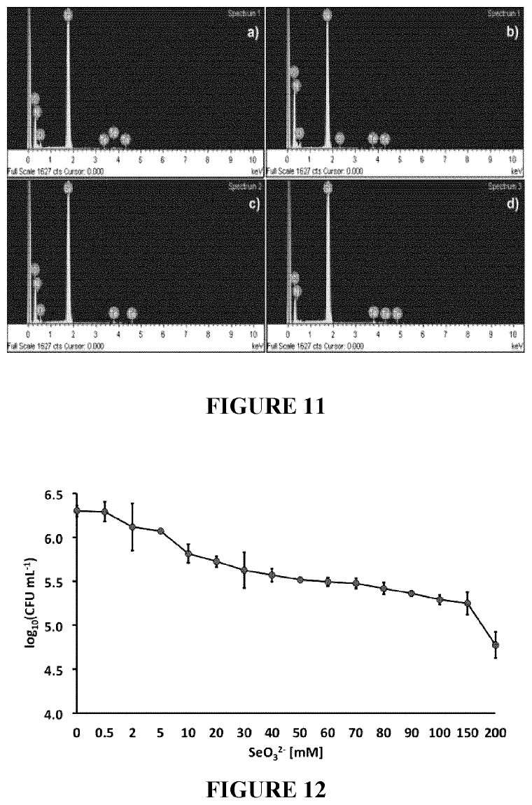

Morphology of TeNRs extracted from BCP1 unconditioned and conditioned cells was evaluated by performing SEM observations (FIG. 10), while the elemental analysis of NRs was performed using Energy-Dispersed X-Ray Spectroscopy (EDX) (FIG. 11 and Table 1). SEM images showed the presence of not aggregated TeNRs surrounded by a dark grey colored material in background (FIG. 10) similarly to TEM observations. In particular, TeNRs₁₀₀ recovered from unconditioned cells underlined the evidence of some NRs forming circular structures around the edge of the surrounding material, while the TeNRs₅₀₀ were homogeneously distributed and had a rod-shaped morphology (FIG. 10a and b). Elemental analysis of TeNRs showed the presence of the same chemical elements for different initial concentrations of the precursor (K₂TeO₃): carbon, nitrogen, oxygen and tellurium (FIG. 11a and b). However, the relative percentage ratios of these elements differed between the TeNRs₁₀₀ and TeNRs₅₀₀. The presence of silicon in the elemental analysis was due to the silicon stubs the samples were mounted onto. Excluding the silicon signal, carbon had the highest percentage value in both TeNRs extracted from unconditioned cells, being 39% (TeNRs₁₀₀) and 49.7% (TeNRs₅₀₀) EDX quantification data showed a higher amount of nitrogen for TeNRs₅₀₀ (9%) as compared to TeNRs₁₀₀ (5%), while oxygen percentage values were comparable for unconditioned TeNRs, yielding 4% (TeNRs₅₀₀) and 3% (TeNRs₁₀₀). Similarly, tellurium amounts were comparable between TeNRs₁₀₀ (4%) and TeNRs₅₀₀ (3%). Moreover, low content of sulfur (0.3%) was detected only in the case of _{TeNRs500} (Table 1). SEM observations of TeNRs produced by conditioned cells revealed morphologies analogous to those seen in unconditioned cells, with the presence of circular organized NRs in the case of TeNRs₁₀₀ and the typical rod-morphology for TeNRs₅₀₀ (FIG. 10c and d). Chemical composition detected by EDX analyses of these nanostructures recovered from conditioned cells indicated the presence of carbon, nitrogen and tellurium (FIG. 11c and d). Carbon showed the highest relative percentage value, being 42% (TeNRs₁₀₀) and 34% (TeNRs₅₀₀), while nitrogen amounts were higher in TeNRs₁₀₀ (7%) than TeNRs₅₀₀ (3%). Moreover, tellurium percentages underlined a relative value of 6% and 3% in TeNRs₅₀₀ and TeNRs₁₀₀, respectively. Finally, only in the case of TeNRs500, EDX data showed the absence of the oxygen signal, which was detected in low content (3%) in TeNRs₁₀₀ (Table 1).

TABLE 1
Elemental Quantification (as Weight Relative Percentage) of
naïve and conditioned TeNRs₁₀₀ and TeNRs₅₀₀.

| | Unconditioned | | Conditioned | |
|---|---|---|---|---|
| | TeNRs₁₀₀ | TeNRs₅₀₀ | TeNRs₁₀₀ | TeNRs₅₀₀ |
| | Weight | Weight | Weight | Weight |
| Element | (Rel. %) | (Rel. %) | (Rel. %) | (Rel. %) |
| Silicon (Si) | 49 | 34 | 45 | 57 |
| Tellurium (Te) | 4 | 3 | 3 | 6 |
| Carbon (C) | 39 | 49.7 | 42 | 34 |
| Oxygen (O) | 3 | 4 | 3 | N.D. |
| Nitrogen (N) | 5 | 9 | 7 | 3 |
| Sulfur (S) | N.D. | 0.3 | N.D. | N.D. |

Elemental quantification is expressed as Weight Relative Percentage of the element detected in the TeNRs samples.
Element not detected are indicated as N.D.

### Discussion

Although Te is a rare natural element in the Earth crust (0.027 ppm) [12], the widespread use of Te-containing compounds in electronics, optics, production of batteries, petroleum refining and mining [38,12,39,40] has led to an increase in its presence in the environment as soluble and toxic oxyanion TeO₃²⁻, causing serious threats to the ecosystem and human health [28]. Interestingly, a large number of Gram-negative [10,11,12,13] and Gram-positive bacteria [16,17,18] were reported to be tolerant and/or resistant towards tellurite. A common strategy used by microorganisms to overcome the toxicity of TeO₃²⁻, relies on the reduction of this oxyanion to its less available/toxic elemental form (Te⁰), producing either intracellular metalloid deposits or nanostructures [12]. In this present study, we have evaluated the capacity of an aerobic Gram-positive Rhodococcus strain, Rh. aetherivorans BCP1, to grow in the presence of high amounts of tellurite (supplied as K₂TeO₃). The results show that under this extreme growth condition, BCP1 cells are able not only to grow significantly but they also reduce TeO₃²⁻ generating intracellular Te-nanostructures, which were isolated and characterized. This result is of some importance since in the past it was reported that oxygen greatly enhances the TeO₃²⁻ toxicity to bacterial cells, i.e. from MIC^{Te} of 250 to 2 μg/mL under anaerobic and aerobic growth, respectively [41]. Conversely, the tolerance of aerobically grown BCP1 strain towards TeO₃²⁻ oxyanions was very high, with a MIC^{Te} value of 2800 μg/mL (11.2 mM). A comparison between BCP1 strain and Gram-positive bacteria described in literature for their ability to grow aerobically in the presence of K₂TeO₃ underlines the high tolerance of Rhodococcus aetherivorans BCP1 strain to this oxyanion. Specifically, bacterial strains such as Lysinibacillus sp. ZYM-1, Bacillus sp. BZ, Corynebacterium difteriaes, Bacillus sp. STG-83, Paenibacillus TeW, and Salinicoccus sp. QW6 were described for their ability to tolerate TeO₃²⁻, with an MIC^{Te} values ranging from 0.8 to 12 mM [19,20,18,21,22,23] (Table 2).

TABLE 2
Comparison of the Minimal Inhibitory Concentration of tellurite
(MIC^{Te}) supplied as potassium tellurite (K₂TeO₃) to rich medium
among Gram-positive bacteria grown under aerobic conditions.

| | MIC^{Te} | |
|---|---|---|
| Strain | [mM] | Literature |
| Salinicoccus sp. QW6 | 12 | Amoozegar et al. (2008) |
| Rhodococcus aetherivorans BCP1 | 11.2 | This study |
| Lysinibacillus sp. ZYM-1 | 2 | Zao et al. (2016) |
| Bacillus sp. STG-83 | 1.25 | Soudi et al.(2009) |
| Corynebacterium difteriaes | 1 | Tucker et al. (1961) |
| Paenibacillus TeW | 1 | Chien et al. (2009) |
| Bacillus sp. BZ | 0.8 | Zare et al. (2012) |

Among the species of Actinomycetales order, BCP1 strain tolerance is therefore ten times higher than the MIC^{Te} (1 mM) of Corynebacterium difteriaes [18]. Conversely, the MIC^{Te} of BCP1 strain was comparable to that obtained with Salinicoccus sp. QW6, which is equal to 12 mM [23]. In this respect, the high tolerance of the BCP1 cells towards TeO₃²⁻ oxyanions under aerobic conditions suggests that this microorganism might play a key role in the in situ and/or ex-situ decontamination procedures of TeO₃²⁻ polluted environments.

In order to evaluate differences in the growth, in the reduction of TeO₃²⁻, as well as in the production of TeNRs by BCP1 strain, unconditioned and conditioned cells were exposed to either 100 or 500 μg/mL (0.4 or 2 mM) K₂TeO₃. The complete reduction of 100 μg/mL TeO₃²⁻ to elemental Te⁰ within 36 h was observed for conditioned BCP1 grown cells as compared to the unconditioned ones (48 h). Similarly, Amoozegar et al. (2008) observed that Salinicoccus sp. QW6 was able to completely reduce 0.5 mM (125 μg/mL) of K₂TeO₃ within 72 h under aerobic conditions. There was no increased removal detected by the QW6 strain at greater concentrations, even after 144 h of incubation [23]. Additionally, an incomplete reduction of TeO₃²⁻ was described by Zare et al. (2012) in the case of Bacillus sp. BZ incubated in Nutrient Broth medium supplemented with 50 or 100 μg/mL (0.2 or 0.4 mM) of K₂TeO₃ within 50 h of exposure [20]. By contrast, when the BCP1 strain was incubated in the presence of 500 μg/mL of K₂TeO₃, the reduction of the initial concentration of TeO₃²⁻ oxyanions resulted to be higher in the case of BCP1 conditioned grown cells (348 μg) rather than the unconditioned ones (218 μg), within 5 culturing days. Nevertheless, an incomplete reduction of the TeO₃²⁻ added (500 μg/mL) was observed. Although cellular thiols (RSH) and glutathione (GSH) molecules are likely to reduce TeO₃²⁻ oxyanions [5] with a consequence of a strong cytoplasmic redox unbalance of the glutathione/glutaredoxin and thioredoxin pool [42,43], it is noteworthy that glutathione molecules are not commonly present in Actinobacteria, except in the case of horizontal gene transfer [44]. In Actinomycetes strains, analogous functions to glutathione (GSH) molecules are performed by mycothiols (MSH; also designated AcCys—GlcN—Ins), which are the major species of thiols present [45]. Similarly to GSHs, MSHs are able to reduce metals and toxic compounds thanks to the presence of thiol groups in cysteine moieties [45], which provide three possible metal ligands (—S—, —NH₂, —COO—). The result of these oxidation-reduction reactions is the production of Reactive Oxygen Species (ROS) e.g. hydrogen peroxide, which cause cellular death [46]. On the other hand, both GSH and MSH molecules are less prone to the oxidation when amino and carboxylic groups are blocked by γ-glutamyl and glycine residues or acetyl and GlcN—Ins, respectively [47,48]. In this respect, the capacity of BCP1 cells to grow aerobically and tolerate high concentrations of tellurite might be due to the greater redox stability of MSHs as compared to GSHs [49], under oxidative stress conditions generated by the simultaneous presence of oxygen and TeO₃²⁻. Moreover, catalase, which is a key enzyme that overcomes cellular oxidative stress, is able to reduce tellurite to its elemental form (Te⁰), conferring the resistance to aerobic microorganisms towards this oxyanion [50]. However, the mechanism of tellurite resistance for Gram-positive bacteria belonging to the order of Actinomycetales is scarcely studied. Nevertheless, it is noteworthy to mention the study of Terai and coworkers (1958), in which a cell free extract of Mycobacterium avium was able to reduce tellurite with a non-specific interaction [51]. Furthermore, among tellurite-resistant Gram-positive bacteria, Bacillus sp. STG-83 was characterized for its ability to reduce these oxyanions using a cytoplasmic tellurite reductase [52], while the product of the genes cysK (cysteine synthase), cobA (uroporphyrinogen-III C-methyltransferase), iscS (cysteine desulfurase) of Geobacillus stearothermophilus V conferred resistance to the Escherichia coli K-12 strain towards potassium tellurite [53,54,55].

The production of intracellular Te-deposits as a consequence of TeO₃²⁻ reduction was earlier described in Gram-positive bacteria such as Paenibacillus TeW and Salinicoccus sp. QW6 [22,23], while Baesman and coworkers reported on the presence of Te-nanostructures in the form of clusters/rosettes accumulated on the outer cell surfaces of B. beveridgei and B. selenitireducens [16,17]. In detail, the Te-nanostructures produced by

Bacillus strains clustered together after their synthesis, forming larger and thicker shard-like structures, which were able to adhere each other and to collapse into bigger rosettes [16,17]. Conversely, our present TEM images of BCP1 unconditioned cells grown in the presence of either 100 or 500 μg/mL of K₂TeO₃ revealed the presence of intracellular stable Te-nanorods (TeNRs), similar to those described by Zare and colleagues in Bacillus sp. BZ [20]. Moreover, TeNRs isolated from unconditioned or conditioned BCP1 cells as seen by TEM and SEM analyses, still appeared in the form of individual and not clustered rod-shaped nanostructures (FIGS. 6 and 10). Isolated TeNRs were embedded into a slightly electron-dense surrounding material, whose organic nature was revealed by signals corresponding to carbon, oxygen, nitrogen and sulfur as detected by EDX spectroscopy. Similar observations were recently obtained by Zonaro and coworkers studying Te-nanoparticles (TeNPs) produced by the Gram-negative Ochrobactrum sp. MPV1 strain [26]. The Zeta potential measurements highlighted a similar negative potential of either studied TeNRs suspensions or the supernatants recovered from Te-nanostructures (FIGS. 7 & 8), reinforcing the indication of an organic material associated with the BCP1 TeNRs, possibly involved in stabilizing these nanostructures, since tellurium does not have a net charge in its elemental state (Te⁰). Our conclusion is also in line with the study by Wang et al. (2006), who ascribed the strong negative surface potential of chemically synthetized Te-nanowires to carboxylic groups of L-cysteine ligands in solution [56]. Moreover, DLS analyses of all studied TeNRs samples showed size distributions that were virtually indistinguishable for TeNRs extracted from BCP1 unconditioned and conditioned grown cells. The only factor that appeared to have an effect on the measured sizes was the initial concentration of TeO₃²⁻ (100 or 500 μg/mL).

Additionally, the size distributions of the analyzed supernatants recovered after removing TeNRs showed peaks slightly shifted towards smaller sizes. These results suggest that the size distributions obtained by DLS for all TeNRs suspensions do not depend only on the presence of the nanorods in the samples. Nanostructures are known to have a high surface energy and may be thermodynamically unstable in suspension [57]. The stability of nano-suspensions is increased if there is an electrostatic repulsion between the particles due to the presence of charges on the surface or if the surface is coated with molecules that prevent the particles to come into close contact with each other and collapse into aggregates [58,59]. The latter form of stabilization, so called steric stabilization, is widely used in chemical synthesis of nanoparticles and nanorods [60]. In the case of TeNRs produced by the BCP1 strain, both electrostatic and steric stabilization seem to play a role. The organic matter surrounding TeNRs is charged as confirmed by Zeta potential measurements. It is important to mention that the presence of the organic surrounding material in solution is essential to the stability of TeNRs. Our attempts to remove it from the nanorods suspensions by several rounds of centrifugation resulted in an irreversible aggregation of the TeNRs. This result combined with the DLS and Zeta potential data suggest that (i) the organic surrounding material is not covalently attached to the surface of TeNRs, and (ii) it is adsorbed on the surface and also present in solution in equilibrium, playing a crucial role in the colloidal stability of TeNRs. We have not been able to confirm the identity of these organic molecules. However, there is a strong possibility that hydrophobic molecules, either lipids or a secreted biosurfactant may be the major constituents of the mixture. There are at least two arguments in favor of this hypothesis. First, due its amphiphilic properties lipids are known to form nanosized aggregates when suspended in aqueous solution. Such nanostructures were observed by DLS even after the nanorods were removed from solution. Second, chemical synthesis of nanorods typically requires the presence of a surfactant at high concentrations to drive their synthesis to one direction [61]. In this regard, Rhodococcus species are known to produce biosurfactant molecules such as trehalose mycolates and glycolipids under physiological and nitrogen limiting growth conditions [62,63], respectively. Therefore, it is reasonable to suggest that the nanorod formation may be mediated by the biosurfactant co-produced by the BCP1 strain.

Due to the presence of TeNRs embedded in an undefined organic material, the actual length of the nanorods was established using ImageJ software based on TEM images. As a result, an incremented length of TeNRs was observed as function of the tellurite concentration (100 or 500 μg/mL of K₂TeO₃), as well as the condition of growth as unconditioned or conditioned cells. In this regard, the dependence of TeNRs length on the initial concentration of the available precursor (TeO₃²⁻) was reported for the production of chemically synthesized nanostructures [64], while the variation of nanorods size as function of the growth conditions (unconditioned or conditioned cells) may be explained by the LaMer mechanism of nanomaterials formation. According to this mechanism, when the reduction of the precursor to its elemental form occurs, a high concentration of monomers in solution is produced, leading to the formation of nucleation seeds that subsequently grow as nanostructures [65]. Most likely, the reduction of the precursor (TeO₃²⁻) by unconditioned BCP1 cells led to the production of a high concentration of monomers (Te⁰ inside the cells, followed by the formation of Te-seeds of nucleation, which finally grew as TeNRs. As a consequence of the unconditioned growth, some Te-seeds of nucleation were still present inside the cells re-inoculated to perform the conditioned growth, which might be used by conditioned cells to produce longer TeNRs.

Several Rhodococcus strains were previously described for their ability to generate metal nanostructures i.e. gold (AuNPs) [66], silver (AgNPs) [67], and zinc oxide (ZnONPs) [68] nanoparticles; however, these rhodococci were scarcely investigated as cell factories for the production of metalloid nanostructures. To the best of our knowledge, this is the first report on the synthesis of rod-shaped nanostructures made of elemental tellurium (TeNRs) by a bacterial strain belonging to the Rhodococcus genus.

### Conclusions

The capacity of the BCP1 strain belonging to Rhodococcus genus to grow aerobically in the presence of high amounts of the toxic oxyanion tellurite and to reduce it into elemental tellurium) (Te⁰) was assessed. In particular, conditioned BCP1 cells were able to reduce a greater amount of TeO₃²⁻ oxyanions at a faster rate as compared to unconditioned cells. The estimated MIC value (2800 μg/mL or 11.2 mM) of TeO₃²⁻ for aerobic growth of BCP1 strain underlined its feature to tolerate high concentration of this toxic oxyanion, as compared to other Gram-positive bacteria previously described as tellurite-tolerant and/or resistant microorganisms. Additionally, the BCP1 strain was able to produce intracellular rod-shaped nanostructures, which did not aggregate. These TeNRs were embedded in an organic surrounding material, showing an increasing length as function of tellurite concentration (100 or 500 μg/mL of K₂TeO₃) and the growth condition such as unconditioned or conditioned cells.

Since tellurium is a versatile narrow band-gap p-type semiconductor [69], this element exhibits unique properties such as photoconductivity, high piezoelectricity, thermoelectricity [70], non-linear optical response [71]. In this respect, TeNRs have found applications as optoelectronic, thermoelectric, piezoelectric devices, as well as gas sensors and infrared detectors [72,73,74,75,76]. Moreover, TeNRs have been investigated for their antibacterial, antioxidant and anticancer properties [77]. Although further investigations are required in order to evaluate the potential use of TeNRs synthetized by Rhodococcus aetherivorans BCP1, the present study demonstrated that aerobically grown BCP1 strain can be utilized as a cell factory for metalloid nanostructure production.

### REFERENCES

[1] Dittmer D C. Tellurium. Chem Eng News. 2003;81:128.

[2] Cairnes D D. Canadian-containing ores. J Can Min Inst. 1911;14:185-202.

[3] Haynes W M. Section 4: Properties of the elements and inorganic compounds. In CRC Handbook of Chemistry and Physics 95th Edition. CRC Press/Taylor and Francis. 2014;p. 115-120.

[4] Cooper W C. Tellurium. Van Nostrand Renhod Co, New York. 1971.

[5] Turner R J. Tellurite toxicity and resistance in Gram-negative bacteria. Rec Res Dev Microbiol. 2001;5: 69-77.

[6] Taylor D E. Bacterial tellurite resistance. Trends Microbiol. 1999;7:111-115.

[7] Nies D. Microbial heavy-metal resistance. Appl Microbiol Biot. 1999;51:730-750.

[8] Harrison J J, Ceri H, Stremick C A, Turner R J. Biofilm susceptibility to metal toxicity. Environ Microbiol. 2004;6:1220-1227.

[9] Jobling M G & Ritchie D A. Genetic and physical analysis of plasmid genes expressing inducible resistance to tellurite in Escherichia coli. Mol Gen Genet. 1987;208:288-293.

[10] Borghese R., Brucale M., Fortunato G., Lanzi M., Mezzi A., Valle F., Cavallini M., Zannoni D. Extracellular Production of Tellurium Nanoparticles by the Photosynthetic Bacterium Rhodobacter capsulatus. Journal of Hazardous Materials, 2016; 309:202-209.

[11] Klonowska A., Heulin T., Vermeglio A. Selenite and Tellurite Reduction by Shewanella oneidensis. Applied and Environmental Microbiology. 2005;71:5607-5609.

[12] Di Tommaso G., Fedi S., Carnevali M., Manegatti M., Taddei C., Zannoni D. The membrane-bound respiratory chain of Pseudomonas pseudoalcaligenes KF707 cells grown in the presence or absence of potassium tellurite. Microbiology. 2002;148:1699-1708.

[13] Turner R J., Weiner J H., Taylor D E. Tellurite-mediated thiol oxidation in Escherichia coli. Microbiology. 1999;145:2549-2557.

[14] Yurkov V V., Beatty J T. Aerobic anoxygenic phototrophic bacteria. Microbiol Mol Bio Rev. 1998; 62(3):695-724.

[15] Yurkov V V., Jappe J., Vermeglio A. Tellurite resistance and reduction by obligately aerobic photosynthetic bacteria. Applied and Environmental Microbiology. 1996;62:4195-4198.

[16] Baesman S M., Stolz J F., Kulp T R., Oremland R S. Enrichment and isolation of Bacillus beveridgei sp. nov., a facultative anaerobic haloalkaliphile from Mono Lake, Calif., that respires oxyanions of tellurium, selenium, and arsenic. Extremophiles. 2009;13:695-705.

[17] Baesman S M., Bullen T D., Dewald J., Zhang D H., Curran S., Islam F S., et al. Formation of tellurium nanocrystals during anaerobic growth of bacteria that use Te oxyanions as respiratory electron acceptors. Applied and Environmental Microbiology. 2007;73:2135-2143.

[18] Tucker F L., Thomas J W., Appleman M D., Donohue J. Complete reduction of tellurite to pure tellurium metal by microorganisms. J. Bacteriology. 1962;83:1313-1314.

[19] Zhao Y., Dong Y., Zhang Y., Che L., Pan H., Zhou H. Draft Genome Sequence of a Selenite- and Tellurite-Reducing Marine Bacterium, Lysinibacillus sp. strain ZYM-1. Genome Announcements. 2016;4:1.

[20] Zare B., Faramarzi M A., Sepehrizadeh Z., Shakibaie M., Rezaie S., Shahverdi A R. Biosynthesis and recovery of rod-shaped tellurium nanoparticles and their bactericidal activities. Mat. Res. Bull. 2012;47:3719-3725.

[21] Soudi M R., Ghazvini P T M., Khajeh K., Gharavi S. Bioprocessing of seleno-oxyanions and tellurite in a novel Bacillus sp. strain STG-83: a solution to removal of toxic oxyanions in presence of nitrate. Journal of Hazardous Materials. 2009;165:71-77.

[22] Chien C C., Han C T. Tellurite restitance and reduction by a Paenibacillus sp. isolated from heavy metal-contaminated sediment. Environmental Toxicology and Chemistry. 2009;28:1627-1632.

[23] Amoozegar M A., Ashengroph M., Malekzadeh F., Razavi M R., Naddaf S., Kabiri M. Isolation and initial characterization of the tellurite reducing moderately halophilic bacterium, Salinicoccus sp. strain QW6. Microbiological Research. 2008;163:456-465.

[24] Turner R J., Borghese R., Zannoni D. Microbial processing of tellurium as a tool in biotechnology. Biotechnology Advances. 2012;30:954-963.

[25] Kim D H., Kanaly R A., Hur H G. Biological accumulation of tellurium nanorod structures via reduction of tellurite by Shewanella oneidensis MR-1. Bioresource Technology. 2012;125:127-131.

[26] Zonaro E., Lampis S., Turner R J., Qazi S J S., Vallini G. Biogenic selenium and tellurium nanoparticles synthetized by environmental microbial isolates efficaciously inhibit bacterial planktonic cultures and biofilms. Frontiers in Microbiology. 2015;6:584.

[27] Ingale A G., Chaudhari A N. Biogenic Synthesis of Nanoparticles and Potential Applications: an EcoFriendly Approach. Journal of Nanomedicine and Nanotechnology. 2013; 4:165.

[28] Das S., Dash H R., Chakraborty J. Genetic basis and importance of metal resistant genes in bacteria for bioremediation of contaminated environments with toxic metal pollutants. Applied Microbiology and Biotechnology. 2016;100:2967-2984.

[29] Lampis S., Zonaro E., Bertolini C., Bernardi P., Butler CS., Vallini G. Delayed formation of zero-valent selenium nanoparticles by Bacillus mycoides SelTE01 as a consequence of selenite reduction under aerobic conditions. Microbial Cell Factories 2014;13:35.

[30] Martinková L., Uhnáková B., Pátek M., Nesvera J., Kren V. Biodegradation potential of the genus Rhodococcus. Environmental International. 2009;35:162-177.

[31] Alvarez H M., Steinbuchel A. Physiology, biochemistry and molecular biology of triacylglycerol accumulation by Rhodococcus. In Biology of Rhodococcus volume 16 of the series Microbiology monographs. Springer Verlag; Heidelberg. 2010;pp 263-290.

[32] Cappelletti M., Presentato A., Milazzo G., Turner R J., Fedi S., Frascari D., Zannoni D. Growth of Rhodococcus sp. strain BCP1 on gaseous n-alkanes: new metabolic insights and transcriptional analysis of two soluble di-iron monooxygenase genes. Frontiers in Microbiology. 2015;6:393.

[33] Frascari D., Pinelli D., Nocentini M., Fedi S., Pii Y., Zannoni D. Chloroform degradation butane-grown cells of Rhodococcus aetherovorans BCP1. Applied Microbiology Biotechnology. 2006;73:421-428.

[34] Cappelletti M., Fedi S., Frascari D., Ohtake H., Turner R J., Zannoni D. Analyses of both the alkB gene transcriptional start site and alkB promoter-inducing properties of Rhodococcus sp. strain BCP1 grown on n-alkanes. Applied Environmental Microbiology. 2011;77:1619-1627.

[35] Orro A., Cappelletti M., D'Ursi P., Milanesi L., Di Canito A., Zampolli J., Collina E., Decorosi F., Viti C., Fedi S., Presentato A., Zannoni D., Di Gennaro P. Genome and Phenotype Microarray Analyses of Rhodococcus sp. BCP1 and Rhodococcus opacus R7: Genetic Determinants and Metabolic Abilities with Environmental Relevance. PLoS ONE. 2015;10(10).

[36] Cappelletti M., Fedi S., Zampolli J., Di Canito A., D'ursi P., Orro A., Viti C., Milanesi L., Zannoni D., Di Gennaro P. Phenotype microarray analysis may unravel genetic determinants of the stress response by Rhodococcus aetherivorans BCP1 and Rhodococcus opacus R7. Research in Microbiology. 2016;1-8.

[37] Turner R J., Weiner J H., Taylor D E. Use of Diethyldithiocarbamate for Quantitative Determination of Tellurite Uptake by Bacteria. Analytical Biochemistry. 1992;204:292-295.

[38] Tang Z., Zhang Z., Wang Y., Glotzer S C., Kotov N A. Self-assembly of CdTe nanocrystals into free-floating sheets. Science. 2006;314:274-278.

[39] Graf C., Assoud A., Mayeasree O., Kleinke H. Solid state polyselenides and polytellurides: a large variety of Se—Se and Te—Te interactions. Molecules. 2009;14:15-

[40] Sen S., Sharma M., Kumar V., Muthe K P., Satyam P V., Bhatta V M., Roy M., Gaur N K., Gupta S K., Yakhmi J V. Chlorine gas sensors using one-dimensional tellurium nanostructures. Talanta. 2009;77:1567-1572.

[41] Borghese R., Borsetti F., Foladori P., Ziglio G., Zannoni D. Effects of the Metalloid Oxyanion Tellurite (TeO32-) on Growth Characteristics of the Phototrophic Bacterium Rhodobacter capsulatus. Applied Environmental Microbiology. 2004;70:6595-6602.

[42] Carmel-Harel 0., Storz G. Roles of the glutathione- and thioredoxin dependent reduction systems in the Escherichia coli and Saccharomyces cerevisiae responses to oxidative stress. Ann. Rev. Microbiol. 2000;54:439-461.

[43] Aslund F., Beckwith J. Bridge over troubled waters: sensing stress by disulfide bond formation. Cell. 1999;96:751-753.

[44] Newton G L., Buchmeier N., Fahey R C. Biosynthesis and Functions of Mycothiol, the Unique Protective Thiol of Actinobacteria. Microbiology and Molecular Biology Reviews. 2008;471-494.

[45] Newton G. L., Arnold K., Price M S., Sherrill C., delCardayre' S B., Aharonowitz Y., Cohen G., Davies J., Fahey R. C., Davis C. Distribution of thiols in microorganisms: mycothiol is a major thiol in most actinomycetes. J. Bacteriol.

1996;178:1990-1995.

[46] Held K D., Biaglow J E. Mechanisms for the oxygen radical mediated toxicity of various thiol-containing compounds in cultured mammalian cells. Radiat. Res. 1994;139:15-23.

[47] Sundquist A R., Fahey R C. The function of gamma-glutamylcysteine and bis-gamma-glutamylcystine reductase in Halobacterium halobium. J. Biol. Chem.

1989;264:719-725.

[48] Newton G L., Bewley C A., Dwyer T J., Horn R., Aharonowitz Y., Cohen G., Davies J., Faulkner D J., Fahey R C. The structure of U17 isolated from Streptomyces clavuligerus and its properties as an antioxidant thiol. Eur. J. Biochem. 1995;230:821-825

[49] Newton G L., Ta P., Fahey R C. A mycothiol synthase mutant of Mycobacterium smegmatis produces novel thiols and has an altered thiol redox status. J. Bacteriol. 2005;187:7309-7316.

[50] Calderón I L., Arenas F A., Pérez J M., Fuentes D E., Araya M A., Saavedra C P., Tantaleán J C., Pichuantes S E., Youderian P A., Vásquez C C. Catalases Are NAD(P)H-Dependent Tellurite Reductases. PLoS one. 2006;1:1-8.

[51] Terai, T., Kamahora, T., Yamamura, Y. Tellurite reductase from Mycobacterium avium. J. Bacteriol. 1958;75:535-539.

[52] Etezad S M., Khajeh K., Soudi M., Ghazvini P T M., Dabirmanesh B. Evidence on the presence of two distinct enzymes responsible for the reduction of selenate and tellurite in Bacillus sp. STG-83. Enzyme and Microbial Technology. 2009;45:1-6.

[53] Vásquez C C., Saavedra C P., Loyola C A., Araya M A., Pichuantes S. The Product of the cysK Gene of Bacillus stearothermophilus V Mediates Potassium Tellurite Resistance in Escherichia coli. Current Microbiology. 2001;43:418-423.

[54] Araya M A., Tantaleán J C., Pérez J M., Fuentes D E., Calderón I L., Saavedra C P., Burra R., Chasteen T G., Vásquez C C. Cloning, purification and characterization of Geobacillus stearothermophilus V uroporphyrinogen-III C-methyltransferase: evaluation of its role in resistance to potassium tellurite in Escherichia coli. Research in Microbiology. 2009;160:125:133.

[55] Tantaleán J C., Araya M A., Saavedra C P., Fuentes D E., Perez J M., Calderón I L., Youderian P., Vasquez C C. The Geobacillus stearothermophilus V iscS Gene, Encoding Cysteine Desulfurase, Confers Resistance to Potassium Tellurite in Escherichia coli K-12. Journal of Bacteriology. 2003;185(9):5831-5737.

[56] Wang Y., Tang Z., Podsiadlo P., Elkasabi Y., Lahann J., Kotov N A. Mirror-like Photoconductive Layer-by-Layer Thin Films of Te Nanowires: The Fusion of Semiconductor, Metal, and Insulator Properties. Advanced Materials. 2006;18:518-522.

[57] Claus P., Hofmeister H. Electron Microscopy and Catalytic Study of Silver Catalysts: Structure Sensitivity of the Hydrogenation of Crotonaldehyde. J. Phys. Chem. B. 1999;103:2766-2775.

[58] Kraynov A., Müller T E. Chapter 12: Concepts for the Stabilization of Metal Nanoparticles in Ionic Liquids. In: Applications of Ionic Liquids in Science and Technology. Edited by Prof. Scott Handy. 2011;p. 235-260.

[59] Aiken J D., Finke R G. A review of modern transition-metal nanoclusters: their synthesis, characterization, and applications in catalysis. Journal of Molecular Catalysis A: Chemical. 1999;145(1):1-44.

[60] Starkey Ott L., Finke R G. Transition-metal nanocluster stabilization for catalysis: A critical review of ranking methods and putative stabilizers. Coordination Chemistry Reviews. 2007; 251:1075-1100.

[61] Rao C N R., Deepak F L., Gundiah G., Govindaraj A. Inorganic Nanowires. Progr. Solid State Chem. 2003;31:5-147.

[62] Rapp P., Bock H., Wray V., Wagner F. Formation, isolation and characterization of trehalose dimycolates from Rhodococcus erythropolis grown on n-alkanes. J. Gen. Microbiol. 1979;115:491-503.

[63] Kim J S., Powalla M., Lang S., Wagner F., Lunsdorf H., Wray V. Microbial glycolipid production under nitrogen limitation and resting cell condition. J. Bacteriol. 1990;13:257-266.

[64] Gautam U K., Rao C N R. Controlled synthesis of crystalline tellurium nanorods, nanowires, nanobelts and related structures by a self-seeding solution process. Journal of Materials Chemistry. 2004;14:2530-2535.

[65] Thanh N T K., Maclean N., Mahiddine S. Mechanisms of Nucleation and Growth of Nanoparticles in Solution. Chem. Rev. 2014;114:7610-7630.

[66] Ahmad A., Senapati S., Khan M I., Kumar R., Ramani R., Srinivas V., Sastry M. Intracellular synthesis of gold nanoparticles by a novel alkalotolerant actinomycete, Rhodococcus species. Nanotechnology. 2003;14:824-828.

[67] Otari S V., Patil R M., Nadaf N H., Ghosh S J., Pawar S H. Green biosynthesis of silver nanoparticles from an actinobacteria Rhodococcus sp. Materials Letters. 2012;72:92-94.

[68] Kundu D., Hazra C., Chatterjee A., Chaudhari A., Mishra S. Extracellular biosynthesis of zinc oxide nanoparticles using Rhodococcus pyridinivorans NT2: Multifunctional textile finishing, biosafety evaluation and in vitro drug delivery in colon carcinoma. Journal of Photochemistry and Photobiology B: Biology. 2014;140:194-204.

[69] Zhao A., Zhang L., Yang Y., Ye C. Ordered tellurium nanowire arrays and their optical properties. Appl. Phys. A. 2005;80:1725-1728.

[70] Araki K., Tanaka T. Piezoelectric and Elastic Properties of Single Crystalline Se—Te Alloys. Japanese Journal of Applied Physics. 1972;11(4).

[71] Tangney P., Fahy S. Density-functional theory approach to ultrafast laser excitation of semiconductors: Application to the A1 phonon in tellurium. Phys Rev B. 2002;14:279.

[72] Suchand Sandeep C S., Samal A K., Pradeep T., Philip R. Optical limiting properties of Te and Ag2Te nanowires. Chemical Physics Letters. 2010;485:326-330.

[73] Sharma Y C., Purohit A. Tellurium based thermoelectric materials: New directions and prospects. J Integr Sci Technol. 2016;4(1):29-32.

[74] MokhtarPanahi-Kalamuei, Mousavi-Kamazani M., Salavati-Niasari M. Facile Hydrothermal Synthesis of Tellurium Nanostructures for Solar Cells. JNS. 2014;4:459-465.

[75] Tsiulyanua D., Marian S., Miron V., Liess H D. High sensitive tellurium based NO2 gas sensor. Sensors and Actuators B. 2001;73:35-39.

[76] Baghchesara M A., Yousefi R., Cheraghizadec M., Jamali-Sheinid F., Saáedi A., Mahmmoudiane M R. A simple method to fabricate an NIR detector by PbTe nanowires in a large scale. Materials Research Bulletin. 2016;77:131-137.

[77] Huang W., Wu H., Li X., Chen T. Facile One-Pot Synthesis of Tellurium Nanorods as Antioxidant and Anticancer Agents. Chem. Asian J. 2016;11:2301-2311.

### Example II

Selenium (Se) was discovered by Jons Jacob Berzelius in 1817 as red-brown precipitate in association with sulfuric acid [1]. It is naturally present in our earth crust as rare element in native rocks and ores, soils, sediments or as association in rare minerals (e.g., crooksite and calusthalite), with concentration ranging from 0.01 to 1200 mg/kg [2,3,4]. Se is an essential micronutrient for living systems as part of the structure of important enzymes, such as glutathione peroxidases and thioredoxin reductases [5,6]. It has multiple beneficial effects due to its presence in the substituted amino acid cysteine as seleno-cysteine, leading in humans to the regulation of at least 25 selenoproteins [7].

Se is a member of the chalcogen family and it belongs to the Group 16 of the periodic table along with Oxygen, Sulfur, Tellurium and Polonium [1]. Since it shares physical-chemical properties with metals and non-metals, Se is normally defined as a metalloid element [1]. The excess presence of Se in the environment is due to anthropogenic activities such as the anode muds produced during the electrolytic refining of copper, the oil refining, and phosphate and metal ore mining [8]. Thanks to its physical-chemical properties (e.g., relatively low melting point, high photo- and semi-conductivity, optical responses and catalytic activity), Se is used in several applications fields: electronic and glass industries, animal feeds and food supplements, metal alloys for batteries, production of pigments and plastics [9,10]. Se is present in the environment in four inorganic forms: Selenate (SeO₄²⁻) and Selenite (SeO₃²⁻) oxyanions, Selenide (Se²⁻), and elemental Selenium (Se⁰) [10]. Among these, SeO⁴⁻ and SeO₃²⁻ are the most toxic and biologically available forms due to their association with Oxygen, which is able to mobilize Se in soils and water, while both Se²⁻ and Se⁰ show lower toxicity levels [11,12]. Selenium is toxic at doses higher than the dietary one (25-30 μg/day), Se-containing compounds represent an important public health concern and efforts have been made to find useable remediation and detoxification approaches [10]. In this sense, it has already been established the existence of several Selenate and/or Selenite-reducing microorganisms able to reduce Se-oxyanions to the less toxic and less bioavailable form of elemental Selenium (SeO) [13], as a bioremediation strategy for the decontamination of Se-polluted environments [14]. Gram-positive bacteria belonging to the genus Bacillus have been largely described for their ability to grow and reduce either SeO₄²⁻ or SeO₃²⁻, such as Bacillus mycoides SelTE01, Bacillus cereus CM100B and Bacillus selenitireducens MLS10 [10, 15, 16]. Pantoea agglomerans UC-32, Stenotrophomonas maltophilia SelTE02 and Shewanella oneidensis MR-1 have been characterized as some of Gram-negative Selenate/Selenite-reducing bacteria [17,18,19]. In several microorganisms, the reduction of SeO₃²⁻ to elemental Selenium)(Se° leads to the formation of metalloid precipitates and/or nanostructures, such as nanoparticles (NPs) or nanorods (NRs) [20]. In general, nanostructures have unique physical and chemical properties, which differ from bulk material, due to their large surface-volume ratio, large surface energy, spatial confinement and reduced imperfections [21]. Thanks to their properties, nanomaterials have been applied in different fields, namely: biomedicine, environmental engineering and agricultural industries [22]. In particular, SeNPs/NRs possess adsorptive ability, antioxidant functions and marked biological reactivity, including anti-hydroxyl radical efficacy and protective effect against DNA oxidation [23,24]. It has been shown that Se-nanostructures can also exert high antimicrobial activity against human pathogenic bacteria, such as Staphilococcus aureus [25]. Se-nanostructures are mostly synthesized using physical or chemical methods, which involve the use of toxic and harsh chemicals, high costs of production and the formation of hazardous wastes that must be disposed [22]. Furthermore, chemically synthesized SeNPs/NRs could be easily subject to photocorrosion [26]. By contrast, the use of biological systems such as Selenate/Selenite reducing bacteria has been seen as a safe, inexpensive and eco-friendly approach to produce Se-nanomaterials [27], allowing at the same time the decontamination of metalloid-polluted environments.

Bacteria strains belonging to the Rhodococcus genus are aerobic non-sporulating microorganisms of particular interest concerning their remarkable capacity to catalyze a very wide range of toxic compounds, as well as their environmental robustness and persistence [28]. Despite the ability of Rhodococcus spp. to degrade xenobiotics along with their physiological adaptation strategies, i.e. cell membrane composition and intracellular inclusions, were largely reported in the literature [29], very little is known about the capability of these microorganisms to resist to toxic metals/metalloids. In this sense, Rhodococcus aetherivorans BCP1 strain, which has been described as hydrocarbon- and chlorinated solvent degrader, as well as for its unique capacity to overcome stress environmental conditions in the presence of a wide range of antimicrobials and toxic metals/metalloids such as tellurite, arsenate and selenite [30,31,32,33,34], it is likely to be an interesting candidate to study. Thus, the present work is aimed to investigate the ability of Rhodococcus aetherivorans BCP1 to survive in the presence of increasing concentrations of selenite and to produce Se-nanostructures. We evaluated the capacity of BCP1 strain to grow and reduce high concentrations of SeO₃²⁻ oxyanions supplied as Na₂SeO₃. SeO₃²⁻ reduction was also assessed after re-inoculation of pre-exposed cells in fresh medium with new addition of Na₂SeO₃ (conditioned cells). Finally, the produced and isolated Se-nanostructures from BCP1 SeO₃²⁻-grown cells were studied through the use of physical-chemical methods.

### Materials and Methods

Bacterial Strain, Growth Media, Culture Conditions

Rhodococcus aetherivorans BCP1 strain (DSM 44980) was pre-cultured in 250 mL Erlenmeyer Baffled Flask for 48 h in 25 mL of Luria-Bertani medium (indicated as LB) [containing (g/L) NaCl, 10; Yeast Extract, 5; Tryptone, 10]. When necessary the medium was solidified by adding 15 g/L of Agar. BCP1 cells were then inoculated (1% v/v) and grown for 120 h in 50 mL of LB broth to which was added either 0.5 mM or 2 mM of Na₂SeO₃. This first bacterial growth is here defined as unconditioned. After the unconditioned growth, BCP1 cells were re-inoculated (1% v/v) and cultured for other 120 h in 50 mL of fresh LB medium supplied with a new addition of either 0.5 or 2 mM of Na₂SeO₃. Here we refer to this bacterial growth as conditioned. Unconditioned or conditioned cultures were incubated aerobically at 30° C. with shaking (150 rpm). Bacterial growth rate was evaluated by spot plate count method every 24 h over the incubation time. LB agar plates containing the spotted cells were recovered for 48 h at 30° C. The number of growing cells is reported as mean the Colony forming Unit (log10[CFU/mL]) with standard deviation. All the reagents were purchased from Sigma-Aldrich®.

Tolerance of the BCP1 Strain Towards SeO32— Oxyanions

The BCP1 strain has been exposed to different concentrations of Na₂SeO₃, ranging from 0.5 to 200 mM as initial concentration to evaluate its tolerance towards these toxic oxyanions. Briefly, after 24 h of exposure to each concentration of Na₂SeO₃ tested, BCP1 cells were serially diluted in sterile saline solution (NaCl 0.9% w/v) and the number of viable cells were determined by spot plates count on LB agar recovery plates. The assay was conducted in triplicate and the viable cell numbers are indicated as the average of the log10[CFU/mL] with standard deviation.

SeO₃²⁻ Consumption Assay

The residual concentration of SeO₃²⁻ oxyanions over the incubation time of BCP1 cells grown in the presence of either 0.5 or 2 mM of Na₂SeO₃ has been evaluated as published elsewhere [20]. Briefly, the reaction mixture was prepared by adding 10 mL of 0.1 M HCl, 0.5 mL of 0.1 M EDTA, 0.5 mL of 0.1 M NaF, and 0.5 mL of 0.1 M disodium oxalate in a 25- to 30 mL glass tube. A 50- to 250 μL of culture broth containing 100 to 200 nmol of SeO₃²⁻ was added to the above-described mixture, along with 2.5 mL of 0.1% 2,3-diaminonaphthalene in 0.1 M HCl. After all the reagents were mixed, the mixture was incubated at 40° C. for 40 min and then it was cooled down to room temperature. The selenium-2,3-diaminonaphthalene complex was extracted in 6 mL of cyclohexane by shaking the reaction mixture for 1 min. The absorbance of the organic phase was read at 377 nm by using a 1 cm path length quartz cuvette (Helima®) and a Varian Cary® 50 Bio UV-Visible Spectrophotometer. Calibration curve was performed using 0, 50, 100, 150, and 200 nmol of SeO₃²⁻ in LB (R2=0.99). The data are reported as mean values (n=3) with standard deviation. All the manipulations were done in the dark and the reagents were purchased from Sigma-Aldrich®.

Preparation, Extraction, and Purification of SeNRs

Se-nanostructures produced by BCP1 cells grown as unconditioned or conditioned in the presence of 0.5 or 2 mM Na₂SeO₃ were extracted as follow: (i) biomasses were collected by centrifugation (3700 rpm) for 20 min after 5 culturing days; the bacterial cell pellets were washed twice with saline solution (NaCl 0.9% W/v) and resuspended in Tris-HCl (1.5 mM) buffer pH 7.4; (ii) bacterial cells were disrupted by ultrasonication at 22 W for 10 min (30 seconds burst interspersed by 30 seconds of pause) on ice (MICROSON™ Ultrasonic Cell Disruptor XL, Qsonica Misonix Inc.); (iii) cellular debris were then separated from Se-nanostructures in the supernatant by a centrifugation step (3700 rpm) for 20 min; (iv) supernatants containing Se-nanostructures were incubated overnight (16 h) at 4° C. with 1-Octanol (Sigma-Aldrich®) in a ratio 4:1; (v) Se-nanostructures were finally recovered by centrifugation (16,000 rpm) for 15 minutes and resuspended in deionized water.

Here we refer to the selenium nanoparticles and/or nanorods produced by the BCP1 strain as SeNPs_{0.5} or SeNPs₂, and SeNRs_{0.5}or SeNRs₂ depending on either the initial concentration of Na₂SeO₃ present in the growth medium or the morphology and shape of these nanostructures.

Dynamic Light Scattering (DLS) and Zeta Potential Measurements

DLS and Zeta potential measurements of SeN Ps and SeNRs generated by BCP1 cells grown as unconditioned or conditioned have been performed using Zen 3600

Zetasizer Nano ZSTM from Malvern Instruments. The hydrodynamic diameter of these Se-nanostructures was established by analyzing 1 mL of each sample in a spectrophotometric cuvette (10×10×45 mm Acrylic Cuvettes, Sarstedt). Zeta potential measurements have been performed using Folded Capillary Zeta Cell (Malvern Instruments), in which 1 mL of each nanomaterial preparation was dispensed, in order to evaluate their surface charge.

Transmission Electron Microscopy (TEM) Analysis

TEM observations of Se-nanostructures isolated from BCP1 cells grown as unconditioned or conditioned have been carried out by mounting 5 μL of each sample on carbon-coated copper grids (CF300-CU, Electron Microscopy Sciences). Then, samples were air dried and observed using Hitachi H7650 TEM. The actual diameter of SeNPs and length of SeNRs was calculated analyzing with ImageJ software 100 randomly chosen nanoparticles and/or nanorods, respectively. BCP1 cells grown in the presence of 0.5 or 2 mM of Na₂SeO₃ for 120 h were negatively stained using a 1% phosphotungstic acid solution (pH 7.3).

Scanning Electron Microscopy (SEM) and Energy-Dispersed X-ray Spectroscopy (EDX) Analysis

Specimen Aluminum stubs (TED PELLA, INC.) were used as supports to mount Crystal Silicon slides (type N/Phos, size 100 mm, University WAFER), in order to perform SEM (Zeiss Sigma VP) and EDX (INCAx-act Oxford Instruments) analyses of 5 μL of each Se-nanostructures preparation extracted from BCP1 cells grown as unconditioned or conditioned in the presence of 0.5 or 2 mM of Na₂SeO₃. In order to perform elemental quantification of selenium nanostructures a single point selection analysis of either selenium nanoparticles (SeNPs) or selenium nanorods (SeNRs) was carried out.

### Results

Tolerance of Rhodococcus aetherivorans BCP1 Towards SeO₃²⁻ Oxyanions

The capacity of the BCP1 strain to tolerate increased concentrations of SeO₃²⁻ oxyanions present in the growth medium (LB), was established by exposing the cells for 24 h to different Na₂SeO₃ concentrations, ranging from 0.5 to 200 mM. The data summarized in FIG. 12 showed the high tolerance of the BCP1 strain towards SeO₃²⁻ oxyanions. Since 2 log reduction in the number of viable cells counted was observed when BCP1 was incubated in the presence of 0.5 mM (2.00·10⁶ CFU/mL) or 200 mM (6.17·10⁴ CFU/mL) of Na₂SeO₃, no Minimal Inhibitory Concentration of selenite (MIC^{Se}) was established within the range of tested concentrations.

Growth and Consumption of SeO32— by BCP1, and Localization of Selenium Nanostructures

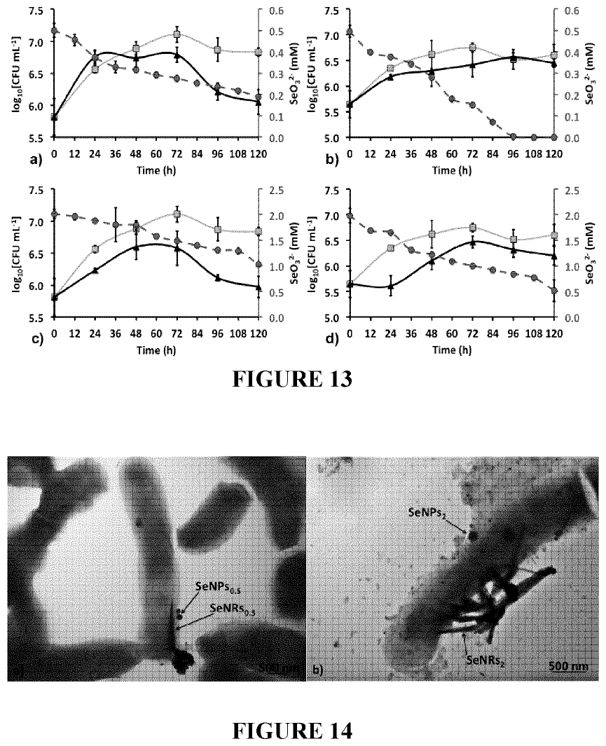

The growth and the consumption rates under either 0.5 or 2 mM of Na₂SeO₃ stress were evaluated for two different physiological states of the BCP1 strain, which are indicated as unconditioned or conditioned grown cells (FIG. 13). Unconditioned BCP1 cells grown in the presence of 0.5 mM Na₂SeO₃ did not show any extended lag phase in growth as compared to the control culture. The consumption of SeO₃²⁻ oxyanions began during the early hours of BCP1 cells incubation (12 h), reducing ≈9% of the initial amount of Na₂SeO₃. BCP1 grown for 72 h reached the highest number of live cells counted (6.33·10⁶ CFU/mL), which corresponded to the stationary phase of growth, while the maximum extent of SeO₃²⁻ consumption (62% of its initial concentration) was observed within 120 h of incubation, although it resulted in an evidence of cell death, being 1.2·10⁶ CFU/mL the number of viable cells (FIG. 13a). By contrast, in the case of conditioned BCP1 cells the reduction of the same amount of SeO₃²⁻ was completed within 96 h of incubation, occurring in the late exponential growth phase. As for unconditioned cells, the conditioned ones did not show any lag phase of growth, entering in the stationary phase after 96 h of incubation (FIG. 13b). In the case of unconditioned BCP1 cells growing in the presence of 2 mM of Na₂SeO₃, no evidence of extended lag phase of growth was observed, and the number of growing cells decreased of 1 log between 72 h (4.24·10⁶ CFU/mL) and 120 h (9.83·10⁵CFU/mL) of incubation, similarly to unconditioned cells grown in the presence of 0.5 mM of Na₂SeO₃.

The consumption/reduction of the oxyanions was not completed over the incubation time (120 h), resulting in the reduction of 50% of the initial amount of SeO₃²⁻. Particularly, the initial concentration of SeO₃²⁻ oxyanions was reduced slowly and constantly by decreasing of 4% every 12 h (FIG. 13c). Considering the BCP1 strain grown as conditioned cells in the presence of 2 mM of SeO₃²⁻ oxyanions, we observed a lag phase of growth of about 24 h and an incomplete reduction of the initial SeO₃²⁻ concentration, although the percentage of residual oxyanions present in the medium was lower (26%) as compared to unconditioned grown cells (50%). Specifically, the initial concentration of SeO₃²⁻ oxyanions was reduced by a 17% and 50% during the lag (24 h) and late exponential (72 h) growth phases, respectively, while at 120 h of incubation the residual oxyanions concentration dropped down to its minimal percentage value (26%) (FIG. 13d).

To detect the production of selenium nanostructures by BCP1, either 0.5 or 2 mM Na₂SeO₃-grown cells for 5 days were negatively stained and analyzed by TEM (FIG. 14). In both cases, the presence of electron-dense selenium nanoparticles (SeNPs) and nanorods (SeNRs) was localized to the outside surface of BCP1 cells.

Dynamic Light Scattering (DLS) Analyses

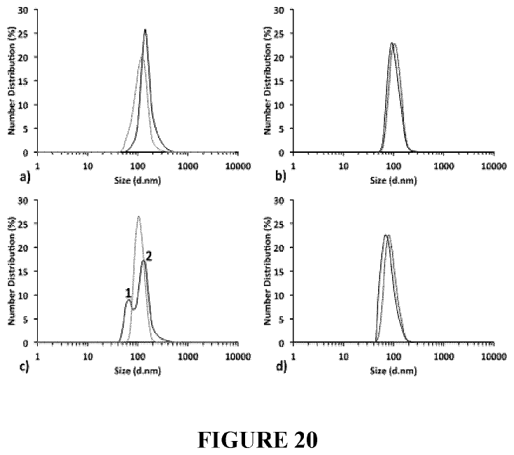

DLS experiments were performed on selenium nanostructures extracted from BCP1 unconditioned and conditioned grown cells (FIG. 20). The measurements yielded distributions of sizes centered at 136±13 nm and 110±24 nm for selenium nanostructures produced and recovered from the BCP1 strain grown as unconditioned cells in the presence of 0.5 mM or 2 mM of Na₂SeO₃, respectively (FIG. 20a). Additional DLS experiments were performed on the supernatants containing selenium nanostructures (FIG. 20b), which were recovered by removing the nanomaterial from the samples through centrifugation at 8000 rpm for 10 minutes. The resulting DLS measurements showed distributions shifted towards smaller sizes compared to the ones obtained from the samples containing the selenium nanostructures, being 101±8 nm (0.5 mM) and 87±7 nm (2 mM). Selenium nanostructures isolated from conditioned BCP1 cells exposed to 0.5 mM of Na₂SeO₃ were featured by two different and discrete peaks centered in size around 80±16 nm (peak 1) and 120±33 nm (peak 2) (FIG. 20c), while a single peak was observed in the case of selenium nanostructures produced by exposing BCP1 cells to 2 mM of SeO₃²⁻ oxyanions, yielding a size distribution of 103±6 nm (FIG. 20c). Supernatants obtained from selenium nanostructures produced by BCP1 conditioned grown cells in the presence of 0.5 or 2 mM of Na₂SeO₃ were investigated performing DLS analyses (FIG. 20d), which resulted in smaller size distributions such as 75±6 nm and 59±9 nm, respectively.

The selenium nanostructure populations were found to be polydisperse as indicated by the values of the measured polydispersity index, being 0.312 or 0.365 for nanomaterial isolated from unconditioned BCP1 cells, and 0.272 or 0.334 for those produced by conditioned ones exposed to 0.5 or 2 mM of SeO₃²⁻, respectively.

Transmission Electron Microscopy (TEM) analysis and Size Distribution of Selenium Nanostructures

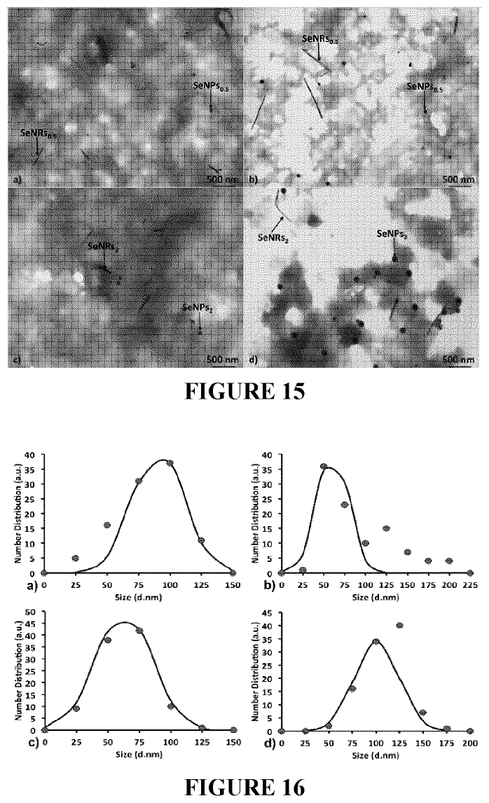

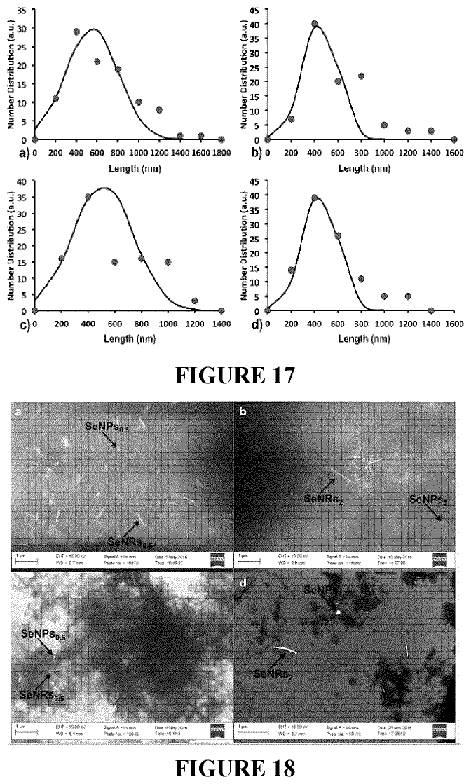

TEM observations were carried out on extracted selenium nanostructures in order to study the size and morphology of the nanomaterials isolated from both unconditioned and conditioned BCP1 cells as product of SeO₃²⁻ reduction (FIG. 15). For each growth mode (unconditioned and/or conditioned grown cells) and concentration of Na₂SeO₃ tested (0.5 and 2 mM), the BCP1 strain was able to synthesize both selenium nanoparticles and nanorods (indicated as SeNPs_{0.5}, SeNPs₂, SeNRs_{0.5} and SeNRs₂ by arrows in FIG. 15a, b, c and d). These nanostructures were not aggregated, polydisperse in solution and surrounded by a slightly electron dense material. The actual diameter and/or length of selenium nanostructures were measured by ImageJ software taking into account 100 randomly chosen SeNPs or SeNRs, respectively. As a result, all SeNPs preparations showed broader distributions (FIG. 16) and those ones recovered from unconditioned BCP1 cells were featured by an average size of 71±24 nm (SeNPs_{0.5}) and 78±42 nm (SeNPs₂), while SeNPs isolated from conditioned cells showed average sizes of 53±20 nm and 97±21 nm for SeNPs_{0.5} and SeNPs₂, respectively. Similarly to SeNPs, SeNRs produced by BCP1 unconditioned grown cells were characterized by broader distributions regarding the measured nanorod lengths (FIG. 17), yielding an average size centered at 555±308 nm and 494±261 nm for SeNRs_{0.5} and SeNRs₂, respectively, while those extracted from conditioned cells were 474±279 nm (SeNRs_{0.5}) and 444±253 nm (SeNRs₂) in length.

Zeta Potential Measurement

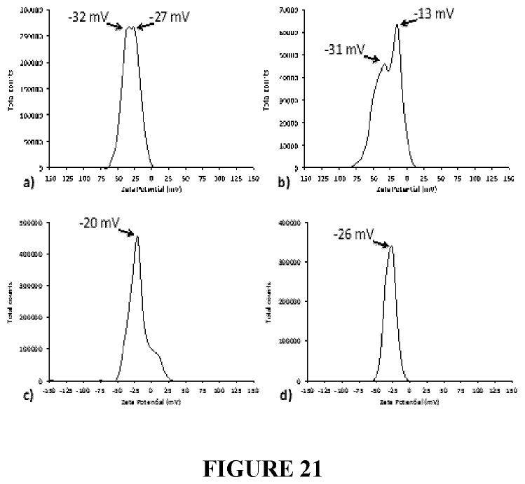

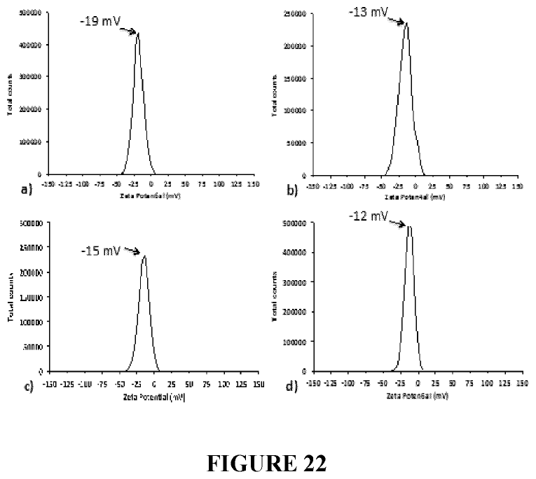

Zeta potential measurements were conducted to evaluate whether the surface of selenium nanostructures was charged (FIG. 21). Two different peaks were detected in Zeta potential plots for both unconditioned selenium nanostructures generated by BCP1 grown in the presence of 0.5 mM (−32 and −27 mV) or 2 mM (−31 and −13 mV) of SeO₃²⁻ oxyanions (FIG. 21a and b). The selenium nanostructures recovered from conditioned BCP1 cells were featured by less negative Zeta potential values, being -20 mV in the case of SeNPs/SeNRS_{0.5} and −26 mV for SeNPs/SeNRs₂ (FIG. 21c and d). Similarly to the DLS analysis, Zeta potential measurements were performed on the supernatants recovered after removing selenium nanostructures by centrifugation (FIG. 22), resulting in less negative surface potentials as compared to those obtained for selenium nanostructure suspensions. The supernatants recovered from the nanomaterials produced by unconditioned cells grown in the presence of either 0.5 or 2 mM of Na₂SeO₃ revealed a surface potential of -19 and -13 mV (FIG. 22a and b), while those obtained from the nanostructures generated by conditioned cells had a surface charge of −15 and −12 mV (FIG. 22c and d), respectively.

Scanning Electron Microscopy (SEM) and Energy-Dispersed X-Ray Spectroscopy (EDX) Analyses

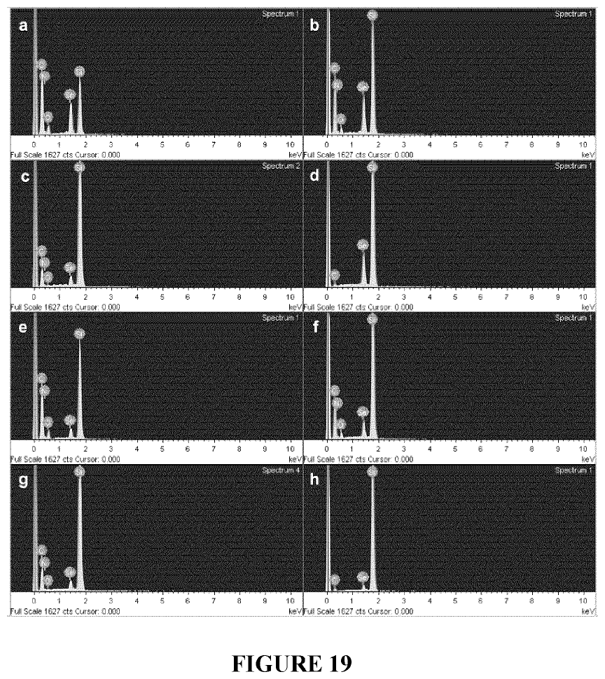

Morphology of selenium nanostructures extracted from BCP1 unconditioned and conditioned cells was evaluated by performing SEM observations (FIG. 18), while the elemental analysis of selenium nanomaterial was performed by Energy-Dispersed X-Ray Spectroscopy (EDX) (FIG. 19; Table 4 and 5). SEM micrographs showed the presence of not aggregated selenium nanostructures isolated from both unconditioned and conditioned BCP1 cells, which were surrounded by material in background similarly to TEM observations. The SEM detected selenium nanomaterials either in the shape of spheres (SeNPs) or rods (SeNRs) homogeneously distributed (indicated by arrows in FIG. 18a, b, c and d). Elemental analysis of SeNPs showed the presence of the same chemical elements for different initial concentrations of the precursor (Na₂SeO₃), namely: carbon, nitrogen, oxygen and selenium (FIG. 19a and b). However, the relative percentage ratios of these elements differed among the SeNPs classes (Table 4). The presence of silicon in the elemental analysis was due to the silicon stubs the samples were mounted onto. Excluding the silicon signal, carbon had the highest percentage value in both SeNPs extracted from unconditioned cells, being 51% (SeNPs_{0.5}) and 48% (SeNPs₂). EDX quantification data showed a higher amount of oxygen (9%) and nitrogen (8%) for SeN PS_{0.5} as compared to SeNPs₂, being 4% and 6%, respectively, while selenium content was comparable between SeNPs_{0.5} (14%) and SeNPs₂ (13%). The chemical composition detected by EDX analyses of

SeNPs recovered from conditioned cells indicated the presence of carbon, nitrogen, oxygen and selenium only for SeN PS_{0.5} (FIG. 19c), while oxygen and nitrogen were not detected in the case of SeNPs₂ (FIG. 19d). Carbon showed a higher relative percentage value for SeNPs_{0.5}(31%) as compared to SeNPs₂ and (11%); on the opposite, selenium amounts were higher in the case of SeNPs₂ (15%) compared to SeNPs_{0.5} (4%). Finally, low contents of oxygen and nitrogen, being 1% and 4%, respectively, were detected for SeNPs_{0.5} (Table 3).

TABLE 3
Elemental Quantification (as Weight Relative Percentage)
of unconditioned and conditioned SeNPs₀₅ and SeNPs₂

| | Unconditioned | | Conditioned | |
|---|---|---|---|---|
| | SeNPs_{0.5} | SeNPs₂ | SeNPs_{0.5} | SeNPs₂ |
| | Weight | Weight | Weight | Weight |
| Element | (Rel. %) | (Rel. %) | (Rel. %) | (Rel. %) |
| Silicon (Si) | 18 | 29 | 60 | 74 |
| Selenium (Se) | 14 | 13 | 4 | 15 |
| Carbon (C) | 51 | 48 | 31 | 11 |
| Oxygen (O) | 8 | 4 | 1 | N.D. |
| Nitrogen (N) | 9 | 6 | 4 | N.D. |

Elemental quantification is expressed as Weight Relative Percentage of the element detected in the TeNRs samples.

Element not detected are indicated as N.D

EDX analysis was also carried out for SeNRs produced by unconditioned and conditioned BCP1 cells. As a result, these nanostructures were featured by the same elements as detected for SeNPs, exception made for SeNRs₂, which showed peaks corresponding only to carbon and selenium (FIG. 19e, f, g and h). SeNRS_{0.5} yielded higher carbon (50%) and nitrogen (7%) relative percentage values as compared to those obtained for SeNRs₂, resulting in 42% and 4%, respectively. Moreover, SeNRs_{0.5} showed similar selenium (5%) and oxygen (7%) contents to those detected in the case of SeNRs2, which were represented by a 6% for both elements (Table 4). Regarding SeNRs isolated from conditioned cells, oxygen and nitrogen were only detected with a low content in the case of SeNRs_{0.5}, resulting in 1% and 5%, respectively. The relative percentage value of selenium was roughly equal between SeNRs_{0.5}(3%) and SeNRs₂ (4%), while high carbon content (34%) featured SeNRs_{9.5} as compared to the 13% recorded for SeNRs₂ (Table 4).

TABLE 4
Elemental Quantification (as Weight Relative Percentage)
of unconditioned and conditioned SeNRs_{0.5} and SeNRs_{2.}

| | Unconditioned | | Conditioned | |
|---|---|---|---|---|
| | SeNRs_{0.5} | SeNRs₂ | SeNRs_{0.5} | SeNRs₂ |
| | Weight | Weight | Weight | Weight |
| Element | (Rel. %) | (Rel. %) | (Rel. %) | (Rel. %) |
| Silicon (Si) | 31 | 42 | 56 | 83 |
| Selenium (Se) | 5 | 6 | 3 | 4 |
| Carbon (C) | 50 | 42 | 35 | 13 |
| Oxygen (O) | 7 | 6 | 1 | N.D. |
| Nitrogen (N) | 7 | 4 | 5 | N.D. |

Elemental quantification is expressed as Weight Relative Percentage of the element detected in the TeNRs samples.

Element not detected are indicated as N.D.

### Discussion

Although a large number of microorganisms have been described for their ability to adsorb and accumulate metals, only few genera of either Gram-positive or -negative bacteria were investigated for their potential in the reduction of metal ions along with the production of nanosized structures [35]. SeNPs production was extensively investigated on anaerobic microorganisms such as Geobacter sulfurreducens, Shewanella oneidensis, Veillonella atypica, Rhodospirillum rubrum, Sulfurospirillum bamesii, Bacillus selenitireducens and Selenihalanerobacter shriftii [36,37,16], to name a few. However, the anaerobic mode of SeNPs production has limitations such as culture conditions, which found biosynthesis optimization processes a very difficult challenge; on the other hand, aerobic bacteria able to tolerate toxic selenium compounds overcome these limitations concerning the biogenically produced selenium-based nanostructures [38]. Strictly aerobic bacteria being part of the Rhodococcus genus have been scarcely investigated regarding both their resistance towards toxic metals/metalloids and the possibility to produce biogenic nanomaterials as product of their hazardous oxyanions reduction. In this respect, the present study highlights the capacity of Rhodococcus aetherivorans BCP1 strain not only to tolerate and grow significantly in the presence of the toxic selenite (SeO₃²⁻) oxyanions under the aerobic growth conditions tested, but also its ability to reduce SeO₃²⁻ generating Se-nanostructures in the form of nanoparticles (SeNPs) and nanorods (SeNRs). The biological significance of these evidences is of some importance considering the enhanced toxicity exerted by SeO₃²⁻ oxyanions upon aerobically grown bacterial cells, i.e. from MIC^{Se} of 4.6 to 1.3 mM under anaerobic and aerobic growth, respectively [39]. Conversely, BCP1 cells grown under aerobic condition showed a high tolerance towards SeO₃²⁻ oxyanions, with a MIC^{Se} value greater than 200 mM (FIG. 12). A comparison between BCP1 and Gram-positive bacteria described in literature for their ability to grow aerobically in complex medium supplied with Na₂SeO₃ underlines its high tolerance to this oxyanion. Specifically, the strain Salinicoccus sp. QW6, several bacterial strains belonging to the Bacillus genus such as, B. sp. STG-83, B. cereus, B. mycoides SelTE01, B. sp. MSh-1, two different B. subtilis strains, B. licheniformis, B. megaterium, as well as three different Actinobacteria being part of the Streptomyces genus named S. bikiniensis strain Ess_amA-1, S. microflavus strain FSHJ31 and S. sp ES2-5 were described for their ability to tolerate SeO₃²⁻, with MIC^{Se} values ranging from 0.8 to 800 mM [5,15,40-49] (Table 5).

TABLE 5
Comparison of the Minimal Inhibitory Concentration of selenite
(MIC^{Se}) supplied as sodium selenite (Na₂SeO₃) to rich medium
among Gram-positive bacteria grown under aerobic conditions.

| | MIC^{Se} | |
|---|---|---|
| Strain | [mW] | Literature |
| Salinicoccus sp. QW6 | 800 | Amoozegar et al. (2008) |
| Bacillus sp. STG-83 | 640 | Soudi et al. (2009) |
| Rhodococcus aetherivorans | >200 | This study |
| BCP1 | | |
| Streptomyces sp. ES2-5 | 50 | Tan et al. (2016) |
| Bacillus mycoides SelTE01 | 15 | Vallini et al. (2005) |
| Bacillus licheniformis | >10 | Dhanjal et al. (2011) |
| Bacillus cereus | >10 | Dhanjal et al. (2010) |
| Bacillus subtilis | >5 | Garbisu et al. (1995) |
| Bacillus sp. MSh-1 | <3.16 | Shakibaie et al. (2010) |
| Streptomyces microflavus | 2.53 | Forootanfar et al. (2014) |
| FSHJ31 | | |
| Bacillus megaterium | 2 | Mishra et al. (1995) |
| Streptomyces bikiniensis | Not | Ahmad et al. (2015) |
| Ess_amA-1 | Determined | |
| Bacillus subtilis | Not | Wang et al. (2010) |
| | Determined | |

Among the species of Actinomycetes listed in Table 5, BCP1 showed tolerance towards SeO₃²⁻ oxyanions of 4 or 80 times higher than the MIC^{Se} evaluated for Streptomyces sp. ES2-5 (50 mM) [49] and Streptomyces microflavus FSHJ31 (2.53 mM) [5], respectively. Moreover, the tolerance of BCP1 towards SeO₃²⁻ oxyanions was of the same order of magnitude to those obtained for Salinicoccus sp. QW6 and Bacillus sp. STG-83 [40,41], suggesting that this microorganism might play a key role in the in situ and/or ex-situ decontamination approaches of SeO₃²⁻ polluted environments.

The growth, the reduction of SeO₃²⁻, as well as the production of Se-nanostructures were evaluated by analyzing two different physiological states of the BCP1 strain i.e. unconditioned or conditioned cells, which were exposed to 0.5 or 2 mM of Na₂SeO₃ over 120 h of incubation, based on three different considerations: (i) there was not significant difference between the number of viable cells counted after 24 h exposure to 0.5 mM (2·10⁶ CFU/mL) or 2 mM (1.72·10⁶ CFU/mL) of SeO₃²⁻ oxyanions; (ii) the highest oxyanion concentration supplied to the growth medium (2 mM corresponds to 223 mg Kg⁻¹) is far above than those evaluated in three different highly contaminated sites, i.e. the southwestern area of Ireland and the San Joaquin Valley in US (above 100 mg Kg⁻¹) [50,51], and the northeastern part of Punjab in India (3.6 mg Kg⁻¹ as mean value) [52]; (iii) as Actinomycetes are known to be slow growing strains, the present study was conducted according to a previous report about the characterization of SeNPs within 120 h of Streptomyces microflavus strain FSHJ31 growth [5], which is phylogenetically correlated to BCP1. The complete reduction of SeO₃²⁻ (0.5 mM) was observed only in the case of BCP1 conditioned growing cells over an incubation time of 96 h, while unconditioned 0.5 mM SeO₃²⁻-grown cells and those unconditioned and conditioned grown in the presence of the highest SeO₃²⁻ concentration tested (2 mM), resulted in an incomplete reduction of the initial SeO₃²⁻ amount, with a higher percentage of reduction regarding the conditioned growth mode (FIG. 13). Most likely, SeO₃²⁻ oxyanions are reduced by cellular thiols [53], leading to a strong cytoplasmic redox unbalance of the glutathione/glutaredoxin and thioredoxin pool [54,55]. The result of the oxidation-reduction reactions mediated by thiol groups is the production of Reactive Oxygen Species (ROS) e.g. hydrogen peroxide, which causes cellular death [56]. This process becomes even more challenging in the case of aerobic bacteria as compared to anaerobic ones, due to the presence of oxygen as electron acceptor instead of SeO₃²⁻, resulting in an enhanced oxidative stress under oxic growth conditions [49]. It is noteworthy that glutathione (GSH) and bacillithiol (BSH) molecules are commonly present in Proteobacteria and Firmicutes, respectively, while Actinobacteria are mainly featured by mycothiols (MSH) [57]. In this respect, the greater redox stability of MSHs as compared to GSHs [58] might explain the capacity of BCP1 cells to grow aerobically and tolerate high concentrations of selenite under oxidative stress conditions, as described for Streptomyces sp. ES2-5 [49]. Since SeO₃²⁻reduction generally does not support the aerobic bacterial growth, the great level of resistance towards SeO₃²⁻ along with their incomplete reduction over the 120 h of incubation may suggest a detoxification mechanism for these oxyanions [59], which is in line with previous studies focused on Streptomyces sp. ES2-5 and Comamonas testosteroni S44 [49,60].

Both anaerobic and aerobic bacterial strains investigated for the production of Se-nanostructures were described to produce mostly spherical polydisperse SeNPs, ranging in size between 50 and 500 nm [61]. The production of smaller SeNPs is a common feature among aerobic bacteria due to the presence of oxygen, which may promote the oxidation of the elemental selenium (Se⁰) with a backward reaction, leading to a slower rate of SeO₃²⁻ oxyanions reduction compared to anaerobic strains [61,62]. On the other hand, the synthesis of SeNRs was reported in the case of Bacillus subtilis, Streptomyces bikiniensis strain Ess_amA-1, Pseudomonas alcaliphila and Ralstonia eutropha [44,48,63,64]. Particularly, a variation in the temperature, in the incubation time or growth mode (i.e., growing or resting cells) led to the production and conversion of SeNPs to SeNRs. In the case of B. subtilis, SeNPs were produced by SeO₃²⁻-grown cells at 35° C. for 48 h, while the synthesis was tuned towards rod-shaped nanostructures by incubating the same batch of cells for further 24 h at room temperature [44]. Streptomyces bikiniensis strain Ess_amA-1 and Pseudomonas alcaliphila were able to synthesize SeNPs after 6 h exposure to SeO₃²⁻, while the transformation to SeNRs was detected after 24 and 48 h of incubation, respectively [48,63]. Regarding Ralstonia eutropha, Srivastava and co-workers (2015) reported its ability, as resting cells, to simultaneously produce both selenium NPs and NRs [64]. Similarly to the aforementioned literature, both SeNPs and SeNRs were detected by TEM observations mainly on the outside surfaces of BCP1 negatively stained cells grown in the presence of either 0.5 or 2 mM of SeO₃²⁻ oxyanions over 120 h of incubation (FIG. 14). TEM and SEM micrographs of Se-nanostructures isolated from unconditioned and conditioned BCP1 cells revealed the presence of polydisperse and stable SeNPs and SeNRs surrounded by a slightly electrondense material (FIG. 15), which was of organic nature as detected by EDX spectroscopy (FIG. 19; Table 3 and 4). Since the natural stability of Se-nanomaterials produced by microorganisms as cell factories was earlier ascribed to the presence of an organic layer with a complex molecular composition (i.e., proteins, peptides, enzymes, reducing cofactors) [65,66], the detected organic material surrounding both SeNPs and SeNRs might play a key role in their stabilization in suspension. Due to peculiar properties of nanomaterials (i.e., high surface-to-volume ratio, high surface area and energy), nanostructures in suspension are featured by a high thermodynamic instability [67]. To overcome such instability, nanomaterials tend to reduce their free energy (AG), leading to their aggregation in suspension [67], which needs to be prevented to take advantages of the singular chemical-physical properties of nanomaterials [68]. The stabilization of chemically synthesized nanostructures is generally achieved through (i) the development of an electrostatic interaction between charged nanomaterials, (ii) the adsorption of polymers on their surfaces acting as spacers (steric interaction), or (iii) a combination of the two aforementioned approaches (electrosteric interaction) [69]. In this regard, the negative Zeta potential values of Se-nanostructures produced by BCP1 (FIG. 21) suggested the existence of an electrostatic repulsion interaction between both SeNPs and SeNRs and, therefore, their stability in suspension [70]. Additionally, DLS analyses and Zeta potential measurements of supernatants recovered from Se-nanostructures highlighted similar size distribution (FIG. 20b and d) and surface potential values (FIG. 22), respectively, compared to those whole nanomaterial samples isolated from both unconditioned and conditioned BCP1 cells. These observations reinforced the indication of an organic material associated with both SeNPs and SeNRs produced by Rhodococcus aetherivorans BCP1, probably involved in their stabilization through the development of an electrosteric interaction. Our results are in line with previous studies, which ascribed a key role to enzymes and proteins in both the production and stabilization of biogenic SeNPs, acting simultaneously as reducing and capping agents [53,71-73]. Moreover, since several bacterial strains are able to produce biological surfactants (biosurfactants) under stress condition of growth [74] and since surfactants act as steric or electrosteric stabilizers of nanomaterials in suspension, the potential involvement of biosurfactants in the natural stability of biogenic nanostructures was recently suggested [75]. In this regard, considering that Rhodococcus species are described as biosurfactants producers (i.e., trehaole mycolates and glycolipids) [76,77], the stabilization of Se-nanostructures might be mediated by biosurfactant molecules co-produced by BCP1.

The formation of SeNPs by unconditioned or conditioned BCP1 cells can be explained by the LaMer mechanism of nanoparticles formation. According to this mechanism, the bacterial cells reduce SeO₃²⁻ oxyanions (precursor) into their elemental forms (Se⁰) with the production of a high concentration of monomers, which led to the formation of Se-nucleation seeds [78]. In order to overcome the high instability, several Se-nucleation seeds collapsed each other (Ostwald ripening mechanism), resulting in the production of bigger SeNPs compared to the Se-seeds [78,79]. Additionally, since SeNPs are featured by high free energy and, therefore, low stability in suspension, they can spontaneously dissolve, leading to the release of Se atoms [80], which might precipitate as nanocrystallinites assembling together in one direction with the formation of SeNRs [81]. According to the actual measured average size and length of SeNPs and SeNRs (FIGS. 16 and 17), respectively, the increase in SeO₃²⁻ oxyanions concentration from 0.5 to 2 mM led to the production of bigger SeNPs, which suggested a direct dependency between their size and the concentration of provided precursor. As a consequence, since the Ostwald ripening mechanism is based on the growth of larger nanostructures at the expense of smaller ones, which are featured by a higher solubility [82], smaller SeNPs dissolve faster in suspension, resulting in a greater number of available Se-atoms and, therefore, in an increased length of the assembled SeNRs. On the opposite, bigger SeNPs are more stable and less prone to dissolve in suspension, leading to the production of shorter SeNRs. In this regard, SeNPs produced by BCP1 cells grown in the presence of the lowest SeO₃²⁻ oxyanions concentration (0.5 mM) are featured by a smaller average size (FIG. 16a and c), corresponding to the growth of longer SeNRs (FIG. 17a and c), while bigger SeNPs (FIG. 16b and d), which resulted from the exposure of BCP1 cells to 2 mM SeO₃²⁻, led to the formation of shorter SeNRs (FIG. 17b and d).

Conclusion

Although bacterial strains belonging to the Rhodococcus genus were previously investigated for the production of gold, silver, zinc oxide, and tellurium nanostructures [83-86], the synthesis of selenium-based nanomaterials was scarcely evaluated among the members of this group. Here, we assessed the capacity of Rhodococcus aetherivorans BCP1 to overcome the toxicity of SeO₃²⁻ oxyanions growing aerobically and reducing them into their less toxic elemental form (Se0). Since the evaluated MIC value of the BCP1 strain towards SeO₃²⁻ oxyanions was high (MIC^{Se}>200 mM), this microorganism may play a potential role in the decontamination of selenite-polluted environments. In all the different tested BCP1 growth modes, the rate of SeO₃²⁻ reduction was higher in the case of conditioned growing cells as compared to those unconditioned. Overall, BCP1 was able to produce spherical and rod-shaped Se-nanostructures (SeNPs/NRs), which were featured by a polydisperse size distribution and stability in suspension, due to the presence of an organic surrounding material. Moreover, the concentration of provided precursor was a crucial parameter influencing the SeNPs size and, therefore, the SeNRs length. Indeed, BCP1 cells grown in the presence of the lowest SeO₃²⁻ concentration tested (0.5 mM) produced smaller SeNPs, which led to the growth of longer SeNRs and vice versa, according to the Ostwald ripening mechanism of nanoparticles formation.

Since BCP1 simultaneously produced both SeNPs and SeNRs, further investigations need to be performed evaluating whether the synthesis of Se-nanostructures can be systematically tuned toward one morphology, along with their potential applications in optics, electronics and nanomedicine (i.e., antimicrobial or anticancer agents).

### References

[1] Haynes W M. Section 4: Properties of the elements and inorganic compounds. In CRC Handbook of Chemistry and Physics 95th Edition. CRC Press/Taylor and Francis. 2014;p.115-120.

[2] Ralston N V C., Ralston C R., Blackwell J L. III, Raymond L J. Dietary and tissue selenium in relation to methylmercury toxicity. Neurotoxicology. 2008;29:802-811.

[3] Keller E A. Chapter 2: Earth Materials and Processes. In Environmental Geology 9th Edition. Prentice Hall. 2000.

[4] Reilly C. Chapter 1: Introduction. In Selenium in Food and Health 2nd Edition. Springer Science Media. 2006; 1-18.

[5] Forootanfar H., Zare B., Fasihi-Bam H., Amirpour-Rostami S., Ameri A., Shakibaie M. Biosynthesis and Characterization of Selenium Nanoparticles Produced by Terrestial Actinomycete Streptomyces microflavus Strain FSHJ31. Research and Reviews: Journal of Microbiology and Biotechnology. 2014;3:47-53.

[6] Messarah M., Klibet F., Boumendjel A., Abdennour C., Bouzerna N., Boulakoud M., El Feki A. Hepatoprotective role and antioxidant capacity of selenium on arsenic-induced liver injury in rats. Exp Toxicol Pathol. 2012;64:167-174.

[7] Mehdi Y., Hornick J., Istasse L., Dufrasne I. Selenium in the Environment, Metabolism and Involvement in Body Functions. Molecules. 2013;18:3292-3311.

[8] Whitten K W., Davis R E., Peck M L. Chapter 6: Chemical Periodicity. In General Chemistry 6th Edition. Saunders College Publishing. 2000; p.927-930.

[9] Craig P J., Maher W. Chapter 10: Organoselenium compounds in the environment. In Organometallic Compounds in the Environment 2nd edition. Jon Wiley and Sons. 2003;p.391-398.

[10] Lampis S., Zonaro E., Bertolini C., Bernardi P., Butler C S., Vallini G. Delayed formation of zero-valent selenium nanoparticles by Bacillus mycoides SelTE01 as a consequence of selenite reduction under aerobic conditions. Microbial Cell Factories. 2014;13:35.

[11] Barceloux D G. Selenium. J Toxicol Clin Toxicol. 1999; 37(2):145-172.

[12] Martens D A., Suarez D L. Selenium speciation of soil/sediment determinated with sequential extractions and hydride generation atomic absorption spectrophotometry. Environ Sci Technol. 1996;31:133-139.

[13] Doran J W. Microorganisms and the biological cycling of selenium. Advanced in Microbial Ecology. 1982; 1-32.

[14] Stolz J F., Basu P., Santini J M., Oremland R S. Arsenic and selenium in microbial metabolism. Annu Rev Microbiol. 2006;60:107-130.

[15] Dhanjal S., Cameotra S S. Aerobic biogenesis of selenium nanosphere by Bacillus cereus isolated from coal mine soil. Microbial Cell Factories. 2010;5(9):52.

[16] Oremland R S., Herbel M J., Blum J S., Langley S., Beveridge T J., Ajayan P M., Sutto T., Ellis A V., Curran S. Structural and spectral features of selenium nanospheres produced by Se-respiring bacteria. Applied and Environmental Microbiology. 2004;70(1):52-60.

[17] Torres S K., Campos V L., Leon C G., Rodriguez-Llamazares S M., Rojas S M., Gonzalez M., Smith C., Mondaca M A. Biosynthesis of selenium nanoparticles by Pantoea agglomerans and their antioxidant activity. J Nanopart Res. 2012; 14:1236-1239.

[18] Antonioli P., Lampis S., Chesini I., Vallini G., Rinalducci S., Zolla L., Righetti P G. Stenotrophomonas maltophilia SelTE02 a new bacterial strain suitable for bioremediation of Selenite-Contaminated Environmental Matrices. Applied and Environmental Microbiology. 2007;6854-6863.

[19] Klonowska A., Heulin T., Vermeglio A. Selenite and Tellurite reduction by Shewanella oneidensis. Applied and Environmental Microbiology. 2005;5607-5609.

[20] Kessi J., Ramuz M., Wehrli E., Spycher M., Bachofen R. Reduction of Selenite and detoxification of elemental selenium by the phototropic bacterium Rhodospirillum rubrum. Applied and Environmental Microbiology. 1999;65(11):4734-4740.

[21] Turner R J., Borghese R., Zannoni D. Microbial processing of tellurium as a tool in biotechnology. Biotechnology Advances. 2012;30:954-963.

[22] Ingale A G., Chaudhari A N. Biogenic Synthesis of Nanoparticles and the Potential Applications: an EcoFriendly Approach. Journal of Nanomedicine and Nanotechnology. 2013;4:165.

[23] Zhang W., Chen Z., Liu H., Zhang L., Gao P., Li D. Biosynthesis and structural characteristics of selenium nanoparticles by Pseudomonas alcaliphila. Colloid Surface B. 2011;88:196-201.

[24] Wang H., Zhang J., Yu H. Elemental selenium at nano size possesses lower toxicity without compromising the fundamental effect on selenoenzymes: comparison with selenomethionine in mice. Free Radic Biol Med. 2007;42:1524-1533.

[25] Tran P A., Webster T J. Selenium nanoparticles inhibit Staphylococcus aureus growth. Int J Nanomed. 2011;6:1553-1558.

[26] Dobias J., Suvorova E I., Bernier-Latmani R. Role of proteins in controlling selenium nanoparticles size. Nanotechnology 2011;22(9pp):195605.

[27] Wadhwani S A., Shedbalkar U., Singh R., Chopade B A. Biogenic selenium nanoparticles: current status and future prospects. Applied Microbiology and Biotechnology. 2016;100:2555-2566.

[28] Martinková L., Uhnáková B., Pátek M., Nesvera J., Kren V. Biodegradation potential of the genus Rhodococcus. Environmental International. 2009;35:162-177.

[29] Alvarez H M., Steinbuchel A. Physiology, biochemistry and molecular biology of triacylglycerol accumulation by Rhodococcus. In Biology of Rhodococcus volume 16 of the series Microbiology monographs. Springer Verlag; Heidelberg. 2010;pp 263-290.

[30] Cappelletti M., Presentato A., Milazzo G., Turner R J., Fedi S., Frascari D., Zannoni D. Growth of Rhodococcus sp. strain BCP1 on gaseous n-alkanes: new metabolic insights and transcriptional analysis of two soluble di-iron monooxygenase genes. Frontiers in Microbiology. 2015;6:393.

[31] Frascari D., Pinelli D., Nocentini M., Fedi S., Pii Y., Zannoni D. Chloroform degradation butane-grown cells of Rhodococcus aetherovorans BCP1. Applied Microbiology Biotechnology. 2006;73:421-428.

[32] Cappelletti M., Fedi S., Frascari D., Ohtake H., Turner R J., Zannoni D. Analyses of both the alkB gene transcriptional start site and alkB promoter-inducing properties of Rhodococcus sp. strain BCP1 grown on n-alkanes. Applied Environmental Microbiology. 2011;77:1619-1627.

[33] Orro A., Cappelletti M., D'Ursi P., Milanesi L., Di Canito A., Zampolli J., Collina E., Decorosi F., Viti C., Fedi S., Presentato A., Zannoni D., Di Gennaro P. Genome and Phenotype Microarray Analyses of Rhodococcus sp. BCP1 and Rhodococcus opacus R7: Genetic Determinants and Metabolic Abilities with Environmental Relevance. PLoS ONE. 2015;10(10).

[34] Cappelletti M., Fedi S., Zampolli J., Di Canito A., D'ursi P., Orro A., Viti C., Milanesi L., Zannoni D., Di Gennaro P. Phenotype microarray analysis may unravel genetic determinants of the stress response by Rhodococcus aetherivorans BCP1 and Rhodococcus opacus R7. Research in Microbiology. 2016;1-8.

[35] Oremland R S., Herbal M J., Blum J S., Langely S., Beveridge T J., Ajayan P M., Sutto T., Ellis A V. Structural and spectral features of selenium nanospheres produced by Se-respiring bacteria. Appl Environ Microbiol. 2004;70:52-60.

[36] Pearce C I., Pattrick R A D., Law N., Charnock J M., Coker V S., Fellowes J W., Oremland R S., Lloyd R. Investigating different mechanisms for biogenic selenite transformations: Geobacter sulfuureducens, Shewanella oneidensis and Veillonella atypica. Environ Technol 2009, 30:1313-1326.

[37] Kessi J., Ramuz M., Wehrli M., Spycher M., Bachofen R. Reduction of selenite and detoxification of elemental selenium by the phototrophic bacterium Rhodospirillum rubrum. Appl Environ Microb 1999, 65:4734-4740.

[38] Prakash N T., Sharma N., Prakash R., Raina K K., Fellowes J., Pearce C I., Lloyd J R., Pattrick R A D. Aerobic microbial manufacture of nanoscale selenium: exploiting nature's bio-nanomineralization potential. Biotechnol Lett. 2009;31:1857-1862.

[39] Bebien M., Chauvin J P., Adriano J M., Grosse S., Vermeglio A. Effect of Selenite on Growth and Protein Synthesis in the Phototrophic Bacterium Rhodobacter sphaeroides. Applied and Environmental Microbiology. 2001;67:4440-4447.

[40] Amoozegar M A., Ashengroph M., Malekzadeh F., Razavi M R., Naddaf S., Kabiri M. Isolation and initial characterization of the tellurite reducing moderately halophilic bacterium, Salinicoccus sp. strain QW6. Microbiological Research. 2008;163:456-465.

[41] Soudi M R., Ghazvini P T M., Khajeh K., Gharavi S. Bioprocessing of seleno-oxyanions and tellurite in a novel Bacillus sp. strain STG-83: a solution to removal of toxic oxyanions in presence of nitrate. Journal of Hazardous Materials. 2009;165:71-77.

[42] Vallini G., Di Gregorio S., Lampis S. Rhizosphere-induced Selenium Precipitation for Possible Applications in Phytoremediation of Se Polluted Effluents. Z. Naturforsch. 2005;60:349-356.

[43] Shakibaie M., Khorramizadeh M R., Faramarzi M A., Sabzevari O., Shahverdi A R. Biosynthesis and recovery of selenium nanoparticles and the effects on matrix metalloproteinase-2 expression. Biotechnol. Appl. Biochem. 2010;56:7-15.

[44] Wang T., Yang L., Zhang B., Liu J. Extracellular biosynthesis and transformation of selenium nanoparticles and application in H2O2 biosensor. Colloids and Surfaces B: Biointerfaces. 2010;80:94-102.

[45] Garbisu C., Gonzalez S., Yang W H., Yee B C., Carlson D. E., Yee A., Smith N., Otero R., Buchanan B B., Leighton T. Physiological mechanisms regulating the conversion of selenite to elemental selenium by Bacillus subtilis. BioFactors. 1995;5:29-37.

[46] Dhanjal S., Cameotra S S. Selenite Stress Elicits Physiological Adaptation, in Bacillus sp. (Strain JS-2). Journal of Microbiology and Biotechnology. 2011;21(11):1184-1192.

[47] Mishra R R., Prajapati S., Das J., Dangar T K., Das N., Thatoi H. Reduction of selenite to red elemental selenium by moderately halotolerant Bacillus megaterium strains isolated from Bhitarkanika mangrove soil and characterization of reduced product. Chemosphere. 2011;84:1231-1237.

[48] Ahmad M S., Yasser M M., Sholkamy E N., Ali A M., Mehanni M M. Anticancer activity of biostabilized selenium nanorods synthesized by Streptomyces bikiniensis strain Ess_amA-1. International Journal of Nanomedicine. 2015;10:3389-3401.

[49] Tan Y., Yao R., Wang R., Wang D., Wang G., Zheng S. Reduction of selenite to Se(0) nanoparticles by filamentous bacterium Streptomyces sp. ES2⊐5 isolated from a selenium mining soil. Microb Cell Fact. 2016;15:157.

[50] Rogers P., Arora S P., Fleming G A., Crinion R., McLaughlin J G. Selenium Toxicity in Farm-Animals—Treatment and Prevention. Irish Vet. J. 1990;43:151-153.

[51] Presser T S., Ohlendorf H M. Biogeochemical cycling of selenium in the San Joaquin Valley, California, USA. Environ. Manage. 1987;11:805-821.

[52] Sharma N., Prakash R., Srivastava A., Sadana U S., Acharya R., Prakash N T., Reddy A V R. Profile of selenium in soil and crops in seleniferous area of Punjab, India by neutron activation analysis. J. Radioanal. Nucl. Chem. 2009;281:59-62.

[53] Kessi J., Hanselmann K W. Similarities between the abiotic reduction of selenite with glutathione and the dissimilatory reaction mediated by Rhodospirillum rubrum and Escherichia coli. J. Biol. Chem. 2004;279:50662-50669.

[54] Carmel-Harel O., Storz G. Roles of the glutathione- and thioredoxin dependent reduction systems in the Escherichia coli and Saccharomyces cerevisiae responses to oxidative stress. Ann. Rev. Microbiol. 2000;54:439-461.

[55] Aslund F., Beckwith J. Bridge over troubled waters: sensing stress by disulfide bond formation. Cell. 1999;96:751-753.

[56] Held K D., Biaglow J E. Mechanisms for the oxygen radical mediated toxicity of various thiol-containing compounds in cultured mammalian cells. Radiat. Res. 1994;139:15-23.

[57] Fahey R C. Glutathione analogs in prokaryotes. BBA-Bioenergetics. 2013;1830(5):3182-3198.

[58] Newton G L., Ta P., Fahey R C. A mycothiol synthase mutant of Mycobacterium smegmatis produces novel thiols and has an altered thiol redox status. J. Bacteriol. 2005;187:7309-7316.

[59] Lovley D R. Dissimilatory metal reduction. Ann. Rev. Microbiol. 1993; 47:263-290.

[60] Zheng S., Su J., Wang L., Yao R., Wang D., Deng Y., Wang R., Wang G., Rensing C. Selenite reduction by the obligate aerobic bacterium Comamonas testosteroni S44 isolated from a metal-contaminated soil. BMC Microbial. 2014;14:204-16.

[61] Shirsat S., Kadam A., Naushad M., Mane R S. Selenium nanostructures: microbial synthesis and applications. RSC Advances. 2015;5:92799-92811.

[62] Husen A., Siddiqi K S. Plants and microbes assisted selenium nanoparticles: characterization and application. Journal of Nanobiotechnology. 2014;12:28.

[63] Zhang W., Chen Z., Liu H., Zhang L., Gao P., Li D. Biosynthesis and structural characteristics of selenium nanoparticles by Pseudomonas alcaliphila. Colloids and Surfaces B: Biointerfaces. 2011;88:196-201.

[64] Srivastava N., Mukhopadhyay M. Green synthesis and structural characterization of selenium nanoparticles and assessment of their antimicrobial property. Bioprocess Biosyst Eng. 2015;38:1723-1730.

[65] Singh P., Kim Y J., Zhang D., Yang D C. Biological Synthesis of Nanoparticles from Plants and Microorganisms. Trend in Biotechnology. 2016;34(7):588-599.

[66] Winkel L H E., Johnson C A., Lenz M., Grundl T., Leupin O X., Amini M., Charlet L. Environmental selenium research: from microscopic processes to global understanding. Environ. Sci. Technol. 2012;46:571-579.

[67] Goldstein A N., Echer C M., Alivisatos A P. Melting in Semiconductor Nanocrystals. Science. 1991;256:1425.

[68] Roco M C. Nanoparticles and nanotechnology research. Journal of nanoparticle research. 1999;1-6.

[69] Segets D., Marczak R., Chaufer S., Paula C., Gnichwitz J F., Hirsch A., Peukert W. Experimental and Theoretical Studies of the Colloidal Stability of Nanoparticles: a General Intrepretation based on Stability Maps. ACS NANO. 2011;5(6):4658-4669.

[70] Hiemenz P C., Rajagolapan R. Chapter 12: Electrophoresis and Other Electrokinetic Phenomena. In: Principles of Colloid and Surface Chemistry 3rd edition. Marcel Dekker Inc. 1997;534-571.

[71] Yee N., Ma J., Dalia A., Boonfueng T., Kobayashi D Y. Se (IV) reduction and the precipitation of Se (0) by the facultative bacterium Enterobacter cloacae SLD1a-1 are regulated by FNR. Appl. Environ. Microbiol. 2007;73:1914-1920.

[72] Ma J., Kobayashi D Y. Yee N. Role of menaquinone biosynthesis genes in selenite reduction by Enterobacter cloacae SLD1a-1 and Escherichia coli K12. Environ. Microbial. 2009;11:149-158.

[73] Lenz M., Klovenbach B., Gygax B., Moes S., Corvini P F X. Shedding light on selenium biomineralization: proteins associated with bionanomaterials. Appl. Environ. Microbiol. 2011;77:4676-4680.

[74] Banat I M., Franzetti A., Gandolfi I., Bestetti G., Martinotti M G., Fracchia L., Smyth T J., Marchant R. Microbial biosurfactants production, applications and future potential. Appl. Microbiol. Biotechnol. 2010;87:427-444.

[75] Singh B R., Dwivedi S., Al-khedhairy A A., Musarrat J. Synthesis of stable cadmium sulfide nanoparticles using surfactin produced by Bacillus amyloliquifaciens strain KSU-109. Colloids Surf. B Biointerfaces. 2011;85:207-213.

[76] Rapp P., Bock H., Wray V., Wagner F. Formation, isolation and characterization of trehalose dimycolates from Rhodococcus erythropolis grown on n-alkanes. J. Gen. Microbiol. 1979;115:491-503.

[77] Kim J S., Powalla M., Lang S., Wagner F., Lunsdorf H., Wray V. Microbial glycolipid production under nitrogen limitation and resting cell condition. J. Bacteriol. 1990;13:257-266.

[78] Thanh N T K., Maclean N., Mahiddine S. Mechanisms of Nucleation and Growth of Nanoparticles in Solution. Chem. Rev. 2014;114:7610-7630.

[79] Gates B., Mayers B., Cattle B., Xia Y. Synthesis and characterization of uniform nanowires of trigonal selenium, Advanced Functional Materials. 2002;12:219-227.

[80] Gates B., Yin Y., Xia Y. A solution-phase approach to the synthesis of uniform nanowires of crystalline selenium with lateral dimensions in the range of 10-30 nm, Journal of the American Chemical Society. 2000;122:12582-12583.

[81] Jeong U., Camargo P H C., Lee YH., Xia Y. Chemical transformation: a powerful route to metal chalcogenide nanowires, Journal of Materials Chemistry. 2006;16:3893-3897.

[82] Cao G. Chapter 2: Physical Chemistry of Solid Surface. In: Nanostructures and Nanomaterials, synthesis, properties and applications. Imperial College Press. 2004;15-48.

[83] Ahmad A., Senapati S., Khan M I., Kumar R., Ramani R., Srinivas V., Sastry M. Intracellular synthesis of gold nanoparticles by a novel alkalotolerant actinomycete, Rhodococcus species. Nanotechnology. 2003;14:824-828.

[84] Otari S V., Patil R M., Nadaf N H., Ghosh S J., Pawar S H. Green biosynthesis of silver nanoparticles from an actinobacteria Rhodococcus sp. Materials Letters. 2012;72:92-94.

[85] Kundu D., Hazra C., Chatterjee A., Chaudhari A., Mishra S. Extracellular biosynthesis of zinc oxide nanoparticles using Rhodococcus pyridinivorans NT2: Multifunctional textile finishing, biosafety evaluation and in vitro drug delivery in colon carcinoma. Journal of Photochemistry and Photobiology B: Biology. 2014;140:194-204.

[86] Presentato A., Piacenza E., Anikovskiy M., Cappelletti M., Zannoni D., Turner R J. Rhodococcus aetherivorans BCP1 as Cell Factory for the Production of Intracellular Tellurium Nanorods under Aerobic Conditions. Microbial Cell Factories. 2016;

### Example III

### Materials and Methods

Bacterial Strain, Growth Media, Exposure Conditions

Rhodococcus aetherivorans BCP1 strain (DSM 44980) was cultured as described elsewhere⁴² whose details are indicated in the Supporting Information. The number of viable cells is reported as average of the Colony Forming Unit (log₁₀[CFU mL⁻¹) for 103 each biological trial (n=3) with standard deviation. All the reagents were purchased from Sigma-Aldrich®.

TeO₃²⁻ Bioconversion Assay

The extent of TeO₃²⁻ removal by BCP1 resting cells during the exposure timeframe was estimated as published elsewhere⁴³ and described in detail in the Supporting Information. The data are reported as mean (n=3) of the percentage value corresponding to TeO₃²⁻ removal over the incubation time with standard deviation. Further, since any statistical difference was observed between the CFU mL⁻¹ counted at the earliest stages of BCP1 resting cells incubation to each oxyanion concentration tested, the specific rate of TeO₃²⁻ bioconversion (expressed as μg mL⁻¹ h⁻¹) was calculated using a linear regression of the data collected over 3 h.

Preparation and Recovery of Te-nanostructure Extracts

To prepare and recover Te-nanostructure extracts produced by BCP1 resting cells, for each exposure time the biomasses were collected by centrifugation (3,700 rpm) for 20 minutes, which were then washed twice with saline solution (NaCl 0.9% w/v) and resuspended in Tris-HCl (1.5 mM) buffer pH 7.4. Bacterial cells were then disrupted by ultrasonication at 22 W for 10 minutes (30 seconds of burst interspersed by 30 seconds of pause) on ice (MICROSON™ Ultrasonic Cell 121 Disruptor XL, Qsonica Misonix Inc.). The cellular debris was separated from Te-nanostructure extracts in the supernatant by a centrifugation step (3,700 rpm) for 20 minutes. Supernatants containing the Te-nanomaterial extracts were incubated overnight (16 h) at 4° C. with 1-Octanol⁴⁴ (Sigma-Aldrich®) in a ratio 4:1 (v/v) and then recovered by centrifugation (16,000 rpm) for 15 minutes. Te-nanostructure extracts were finally suspended in deionized water.

Transmission Electron Microscopy (TEM) Characterization of Te-nanostructure Extracts Generated by BCP1 Resting Cells

TEM images of TeO₃²⁻ -exposed BCP1 resting cells, as well as all Te-nanostructure extracts, were captured using a Hitachi H7650 TEM. Additionally, both Bright Field (BF) and High-Resolution (HR) TEM, as well as the corresponding Selected Area Electron Diffraction (SAED) pattern of TeNRs, were collected by FEI Tecnai F20 TEM at an acceleration voltage of 200 kV. TEM samples were prepared by mounting 5 μL of either cellular suspensions or Te-nanostructure extracts on carbon coated copper grids (CF300-CU, Electron Microscopy Sciences), which were then air-dried prior the imaging. TEM micrographs were analyzed through ImageJ software to measure the actual TeNRs length, which was calculated taking 100 randomly chosen nanorods contained in each extract. The distribution was fitted to a Gaussian function to yield TeNRs average length.

Fluorescence Correlation Spectroscopy (FCS) Analysis

The lipophilic tracer 3,3′-dioctadecyloxacarbocyanine perchlorate (DiOC18(3) Invitrogen™)⁴⁵ dissolved in methanol was used as the probe in all experiments. The TeNRs extract recovered from BCP1 resting cells exposed for 16 h to 1000 μg mL⁻¹ was labeled with the dye previously dried under Argon flow, incubating 3 mL of the sample for 30 minutes at room temperature with shaking. FCS experiments were carried out with an ISS Alba IV Confocal Spectroscopy & Imaging Workstation coupled with a Nikon Eclipse Ti-U microscope. The lipophilic tracer was diluted to a final concentration of 2 nM, and 400 μl of this dilution were used to perform FCS. The autocorrelation curves corresponding to both the samples were obtained from 15 independent runs by exiting the dye with a single photon CW Ar-laser (λex=488 nm). The autocorrelation functions were built by the VistaVision ISS software and fitted according to a 3D Gaussian theoretical model of free diffusion⁴⁶ to extract the diffusion coefficients.

Measurement of Electrical Resistance

The evaluation of TeNRs extract's electrical properties, as well as the one of the material surrounding them, which was recovered by a centrifugation step performed at 8,000 rpm for 10 minutes, was carried out by air drying 800 μL of sample onto a 2×1 cm Crystal Silicon wafer (type N/Phos, size 100 mm, University Wafer), which was then used for a Four Probe electrical conductance experiment⁴⁷ at room temperature. The obtained resistance values were recorded using a 5492B Digit Multimeter (BK PRECISION®159), which correspond to the average of 6 independent measurements with standard deviation. The resistance (R) values are expressed as Ohm (Ω), while the electrical conductivity is reported as Siemens per meter (S m⁻¹).

Results and Discussion

The exploitation of bacteria bioconverting chalcogen oxyanions¹¹ is now recognized as a valuable approach to develop green-synthesis strategies to produce unique nanoscale materials⁴⁸. In a previous study, the capability of aerobically BCP1 cells grown in the presence of TeO₃²⁻ to produce

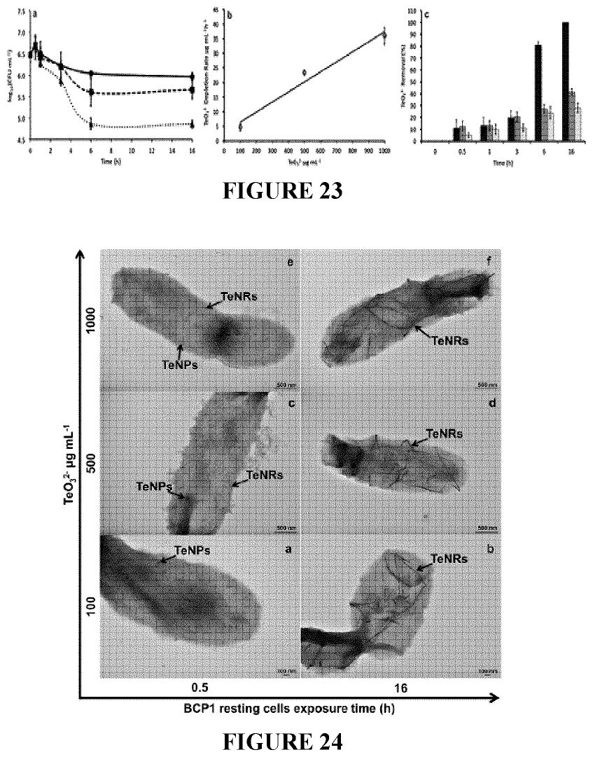

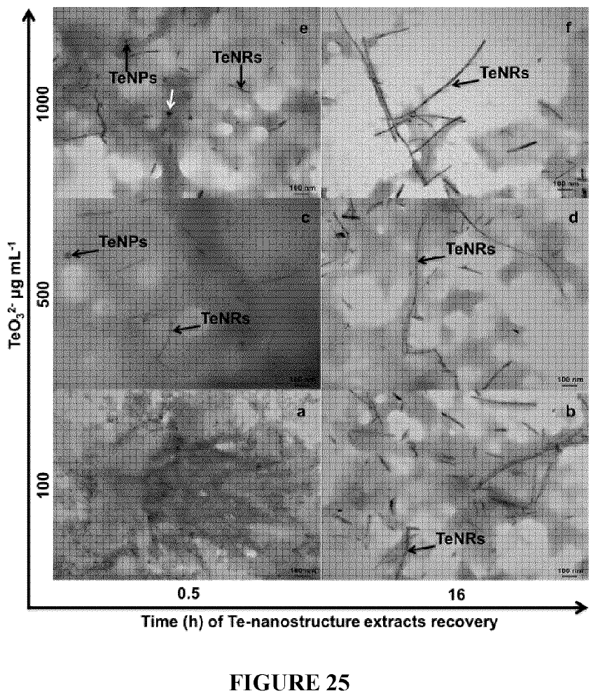

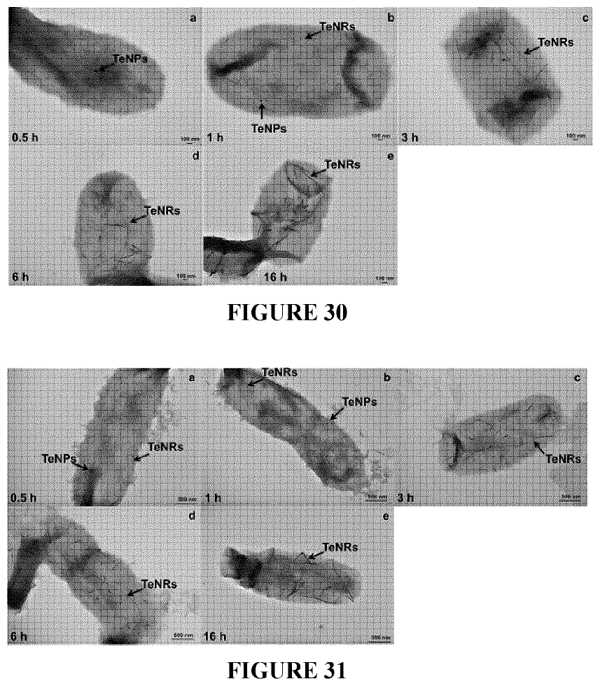

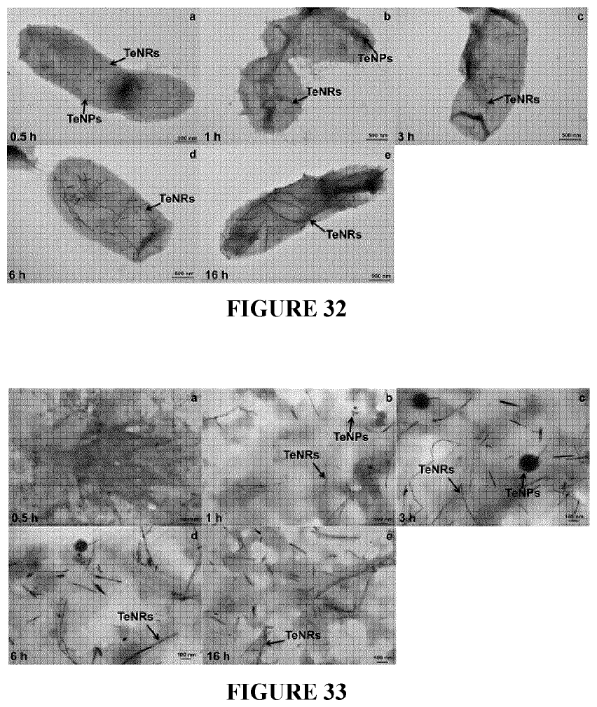

TeNRs upon TeO₃²⁻ bioconversion was observed⁴². Here, the suitability of this strain to generate biogenic TeNRs upon a change of its physiological state (i.e. resting cells) was assessed, underling the greater performance of non-growing cells to produce extremely long TeNRs as compared to actively growing cultures⁴². Indeed, despite the toxicity exerted by TeO₃²⁻that led to a cell death directly proportional to the initial concentration of oxyanions (FIG. 23 1a), 100 μg mL-1 of TeO₃²⁻ were bioconverted 20 h faster by BCP1 resting cells (1.04×10⁶ CFU/mL) (FIG. 23c) as compared to the growing ones (2.67×10⁶ CFU/mL)⁴². Similar conclusions can be drawn in the case of BCP1 resting cells exposed to 500 μg mL⁻¹ of TeO₃²⁻, even though 16 h exposure did not lead to 100% bioconversion (42±3%). BCP1's tolerance towards TeO₃²⁻ was further highlighted by its capability to remove 28±4% (corresponding to 280±40 μg mL⁻¹) when exposed to 1000 μg mL⁻¹ tellurite over 16 h (FIG. 23 c). In comparison, Escherichia coli K12 strain showed a similar TeO₃²⁻ bioconversion trend under anoxic conditions and upon addition of the quinone mediator lawsone⁴⁹, while highly resistant Gram-positive aerobic bacteria such as Bacillus sp. BZ and Salinicoccus sp. QW6 did not bioconvert more than 100 or 125 μg mL⁻¹ of tellurite within 50 or 72 h exposure, respectivelyl^{15,17}. Since the survival extent of BCP1 resting cells for each experimental condition was comparable within 3 h of TeO₃²⁻ exposure, its removal rate was calculated considering the earliest stages of cells incubation, which was 4.6±1.3 μg mL⁻¹h⁻¹ (100 μg mL⁻¹), 23.4±0.7 μg mL⁻¹ h⁻¹ (500 μg mL-1183) and 36±3.0 μg mL⁻¹ h⁻¹ (1000 μg mL⁻¹) (FIG. 23 b), showing a linear correlation as function of the initial TeO₃²⁻ concentration. Finally, no abiotic TeO₃²⁻ removal was observed over the same timeframe, as shown in FIG. 29 the highest TeO₃²⁻ concentration (1000 μg mL-1) tested in this study has been incubated at 30° C. with shaking over a specific timeframe (0, 0.5, 1, 3, 6 and 16 h) either in Phosphate Buffer Saline (PBS) or PBS containing autoclaved biomass (sterile control), to evaluate whether during the exposure time an abiotic loss of the oxyanion supplied to BCP1 resting cells occurred. BCP1's remarkable potential in removing TeO₃²⁻ 187 was also coupled to its proficiency to generate intracellular Te-nanostructures in the form of NPs and NRs in all experimental conditions tested (i.e., TeO₃²⁻ concentration and exposure time) (FIG. 24; FIGS. 30, 31 and 32). In FIGS. 30, 31, and 32, the complete time course of formation and growth of Te-nanostructures is shown, which occurred within BCP1 resting cells as function of either the initial oxyanion concentration tested (100, 500 and 1000 μg mL⁻¹) or the cell exposure time (0, 0.5, 1, 3, 6 and 16 h). In this regard, although several Gram-positive bacterial strains were recently described for their capability to form Te-nanomaterials as NRs, they mostly appeared as needle-like structures and either intra- or extra-cellular clusters or rosettes constituted by TeNRs adhering to each other^{13,50}. Conversely, the production of not aggregated intracellular TeNRs was only observed in the case of Bacillus sp. BZ17, BCP1 growing⁴² and resting cells (FIG. 24). Remarkably, TeNRs within the extracts recovered from BCP1 cells either grown⁴² or exposed to TeO₃²⁻ still maintained their strong thermodynamic stability, even after mounting and air-drying on a carbon-coated copper grid for TEM imaging (FIG. 25; FIGS. 33, 34 and 35).

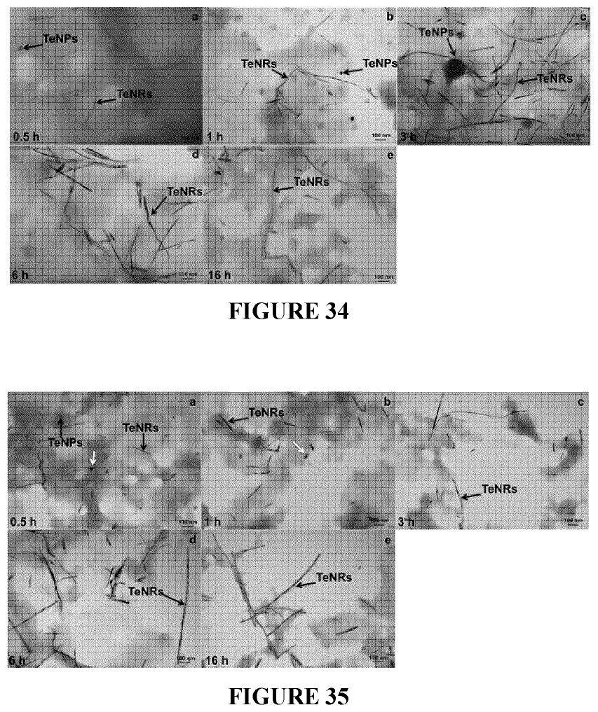

Under resting cell experimental conditions, a progressive shift in the Te-nanostructure morphologies generated by BCP1 was observed. Indeed, during the earliest stage of incubation (0.5 h), the BCP1 strain exposed to the lowest TeO₃²⁻ concentration (100 μg mL⁻¹) displayed primarily TeNPs (FIG. 24a), while at higher initial TeO₃²⁻ concentrations (i.e., 500 and 1000 μg mL⁻¹), both TeNPs and TeNRs were detected within the cells (FIG. 24c and e). TeNPs were still observed up to 1 h of BCP1 resting cells exposure to TeO₃²⁻ precursor (FIGS. 30 b, 31 b and 32 b); while the production of Te-nanomaterials shifted towards a one-dimensional (1D) nanomorphology (TeNRs) when BCP1 cells were exposed to TeO₃²⁻ for more than 3 h (FIG. 24b, d and f; FIGS. 30, 31 and 32.). Similarly, TEM micrographs of Te-nanostructure extracts recovered from BCP1 resting cells exposed over for 0.5 h to 100 μg mL⁻¹ of TeO₃²⁻ displayed the presence of undefined electron-dense nanomaterials resembling NPs (FIG. 25a), while defined TeNPs and TeNRs (FIG. 25c and e) were observed as the concentration of TeO₃²⁻ precursor increased (500 and 1000 μg mL⁻¹). Shard-like NPs were also detected along with TeNRs within Te-nanostructure extracts isolated from BCP1 cells exposed for either 0.5 or 1 h to 1000 μg mL¹ of TeO₃²⁻, as indicated by white arrows in TEM micrographs (FIG. 25e; FIG. 35a and b). Furthermore, after 3 h exposure to either 100 or 500 μg mL⁻¹ of TeO₃²⁻ , Te-nanostructure extracts derived from BCP1 resting cells were featured by the presence of not only TeNRs, but also larger TeNPs as compared to the ones present within the extracts recovered from the cells at their earliest incubation stage (FIGS. 33c and 34c). Finally, although different shapes of Te-nanomaterials within the extracts were detected by TEM, the biosynthesis was tuned towards TeNRs as the main product after either 6 h of cell exposure to 100 and/or 500 μg mL-1 of TeO₃²⁻or 3 h incubation with 1000 μg mL⁻¹ of oxyanion (FIGS. 33, 34 and 35). FIGS. 33, 34, and 35 show a time course experiment carried out on the recovered biogenic Te-nanomaterial extracts from BCP1 resting cells exposed to the different concentration of TeO₃²⁻ tested for different times, in order to evaluate changes in the nanomorphology of Te-nanostructures as function of both TeO₃²⁻ concentration and cell exposure time.

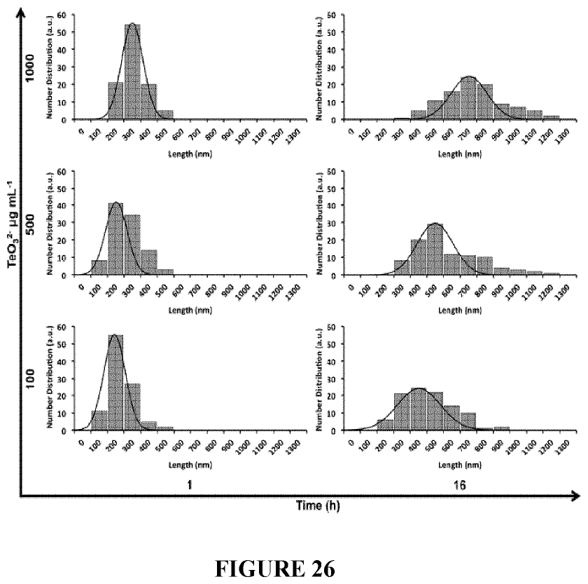

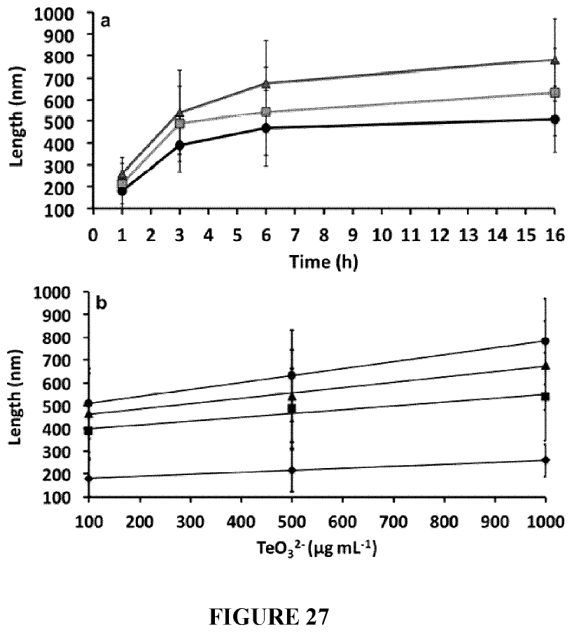

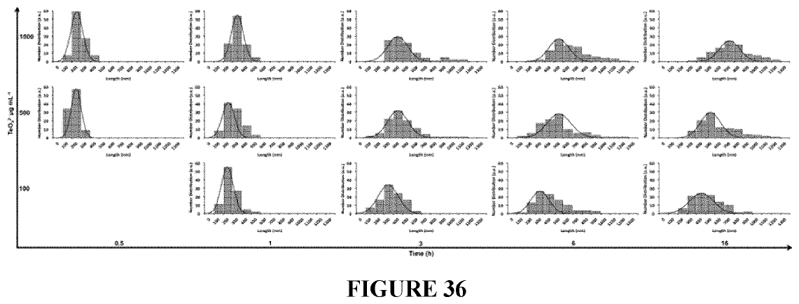

Since TeNRs were the predominant morphology of Te-nanostructures detected by TEM, the measurement of their average length and diameter has been evaluated as function of both BCP1 resting cells exposure time and initial TeO32-concentration (FIG. 26; FIG. 36; Table 6 and 7). TeNRs appeared to be polydisperse in dimension, showing a shift of the NRs length distribution from short to very long ones, as both the exposure time of BCP1 resting cells and the initial TeO₃²⁻ concentration increased. Overall, the average TeNRs length grew exponentially over the time (FIG. 27a), while a linear correlation between the TeNRs average length and the initial TeO₃²⁻ precursor concentration was observed (FIG. 27b). On the other hand, neither the initial TeO₃²⁻concentration nor the exposure time to the oxyanions influenced the measured TeNRs average diameter (Table 7). Although TeNRs average diameter doubled in size from 5±2 nm to 10±3 nm (Table7), there was not a spread generating a two-dimensional (2D) sheet, but instead TeNRs growth primarily was maintained in 1D, producing very long rod- or ribbon-like structures. Further, BCP1 resting cells generated extremely long TeNRs (781±189 nm) as compared to those produced by growing cells (463±147 nm)⁴² , as well as other bacterial strains, such as Rhodobacter capsulatus (369±131 nm)¹³, Bacillus selenitireducens (200 nm)⁵° and Shewanella oneidensis MR-1 (100-200 nm)⁵¹, confirming the greater potentiality of BCP1 non-growing cells as biofactory for TeNRs production.

TABLE 6
TeNRs average length (nm) produced by
Rhodococcus aetherivorans BCP1 resting cells.
TeNRs average length (nm) per initial TeO₃²⁻
concentration [μg mL⁻¹]

| Time (h) | 100 | 500 | 1000 |
|---|---|---|---|
| 0.5 | N.M. | 123 ± 49 | 185 ± 66 |
| 1 | 181 ± 79 | 214 ± 92 | 260 ± 72 |
| 3 | 388 ± 120 | 488 ± 174 | 539 ± 192 |
| 6 | 468 ± 174 | 543 ± 201 | 677 ± 195 |
| 16 | 509 ± 153 | 632 ± 201 | 781 ± 189 |

Average length not measured is indicated as N.M.

TABLE 7
TeNRs average diameter (nm) produced by
Rhodococcus aetherivorans BCP1 resting cells.
TeNRs average diameter (nm) per initial TeO₃²⁻
concentration [μg mL⁻¹]

| Time (h) | 100 | 500 | 1000 |
|---|---|---|---|
| 0.5 | N.M. | 5 ± 2 | 6 ± 2 |
| 1 | 7 ± 2 | 6 ± 2 | 8 ± 2 |
| 3 | 9 ± 3 | 8 ± 2 | 8 ± 2 |
| 6 | 9 ± 3 | 9 ± 2 | 9 ± 3 |
| 16 | 10 ± 3 | 10 ± 3 | 10 ± 3 |

Average length not measured is indicated as N.M.

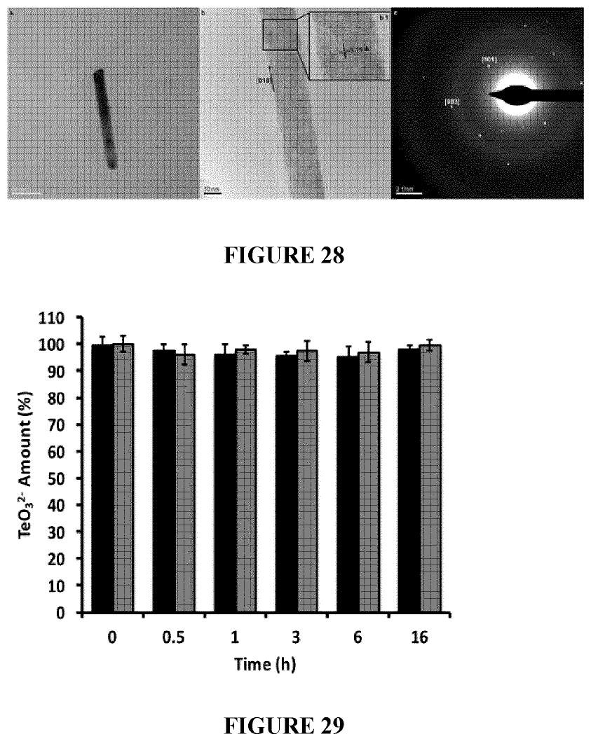

Te⁰ tendency to form 1D nanostructures relies on the high thermodynamic stability of trigonal tellurium (t-Te), which is responsible for the anisotropic growth of Te-nanocrystallinities along one axis52. In this respect, the biogenically synthesized TeNRs analyzed 239 performing BF- and HR-TEM imaging, as well as SAED revealed individual, regular NRs without any defects or dislocations along the longitudinal c-axis, indicating their uniform and single-crystalline nature (FIG. 28a and b). The electron diffraction (ED) patterns collected from different regions of a single TeNR were the same, confirming the unique nature of such biogenic nanomaterials, which resembled the one described for chemical TeNRs⁵³. The periodic fringe spacing of ca. 3.79 A was determined by HR TEM image (FIG. 28b), which is consistent with the established interplanar distance of ca. 3.90 A for the separation between the [010] lattice planes of t-Te [space group P3₁21(152)]⁵³ . Further, the TeNR ED pattern was indexed as pure t-Te phase with calculated lattice constants a=4.38 Å and c=5.83 Å (FIG. 28 c), whose values are in good agreement with the ones reported in the literature (a=4.45 Å; c=5.92 Å; JCPDS 36-1452).

The nanomorphological change observed for Te-nanostructures generated by BCP1 resting cells exposed to TeO₃²⁻ suggested a specific intracellular mechanism of NRs assembly/growth exploited by this bacterial strain, which firstly involved TeNPs formation. According to the established chemical models of TeNRs synthesis⁵⁴⁻⁵⁶, the formation of such 1D nanostructures is preceded by the generation of TeNPs generally featured by an amorphous crystalline structure (a-Te), which confers to these nanoscale materials a high surface energy, resulting in their rapid dissolution and in the availability of Te⁰ atoms in the reaction system⁵⁷. Thus, to overcome their thermodynamic instability, Te⁰ atoms organize themselves depositing as trigonal crystalline (t-Te) Te-nucleation seeds, which then grow in one direction forming NRs through a ripening process^{67,68}. Transposing this chemical model of TeNRs formation to the biological system analyzed in this study, the process resulted to be emphasized, as the TeO₃²⁻ bioconversion occurred in the cytoplasm, leading to a large Te⁰ atom content restricted to the small cellular volume, which will be then available for TeNRs production. During TeNRs chemical synthesis process, the transformation of a-Te within TeNPs into t-Te usually occurs right after the formation of NPs, even though the kinetics of this event is directly dependent on the concentration of TeO₃²⁻ precursor supplied, resulting in a faster dissolution of TeNPs as the initial amount of oxyanion increases^{57,58}. These observations are in line with the results obtained in our study, where the presence of TeNRs was already detected within BCP1 resting cells exposed for 0.5 h to either 500 or 1000 μg mL-1 of TeO₃²⁻ (FIG. 24c and e), while only TeNPs were observed within bacterial cells incubated for the same timeframe to the lowest oxyanion concentration (100 μg mL⁻¹) (FIG. 24a). As a consequence, the fast TeNPs dissolution at high concentration of TeO₃²⁻ precursor led to the production of longer TeNRs when BCP1 resting cells were exposed to increasing TeO₃²⁻ concentrations (FIG. 26 and FIG. 27 a; Table 6). Similarly, the average length of biogenically produced TeNRs increased as BCP1 exposure time to TeO₃²⁻ increased (FIG. 26 and FIG. 27b; Table 6), providing evidence that the process followed a first-order kinetics relative to the number of Te⁰ atoms available, which would elongate pre-existent NRs. This follows closely the growth mechanism of chemically synthesized TeNRs proposed by Liu et al. (2003), where the evolution from TeNPs present at the earliest reaction stage to a mixture of both NPs and NRs (3 h of incubation), and to pure TeNRs after 24 h synthesis was observed⁵⁸.

Chemical synthesis of NRs is mostly reliant on the addition to the reaction system of surfactant molecules⁵⁹⁻⁶¹, which strongly bind and adsorb onto the surface of the nanomaterials⁵⁸. According to the TeNRs surfactant-assisted growth proposed by Liu and co-authors (2003), during the first stage of the reaction surfactant molecules interact with TeNPs limiting the aggregation of Te⁰ atoms and, therefore, mediating the production of stabilized NPs. Once the transition from a-Te to t-Te takes place, t-Te atoms grow generating single-crystalline NRs, whose formation is driven by the surfactant molecules present in solution facilitating one directional growth of the nanomaterial⁵⁸. Thus, the strong interaction between surfactants and TeO 286 atoms in a nanoparticle confines the growth of TeNRs only in one plane, allowing their deposition along one axis, which results in the formation of nanostructures featured by a constant diameter^{57,58,62,63}. As for chemically synthesized TeNRs, those produced by BCP1 resting cells showed average diameters that did not drastically change as function of TeO₃²⁻ precursor concentration or exposure time, ranging from 5±2 nm to 10±3 nm, which is in line with those calculated by Liu and co-workers for TeNRs synthesized by using chemical surfactants⁵⁸. Moreover, surfactants used in TeNRs chemical synthesis act also as their stabilizing agents, providing both the steric effect arising from their alkyl chains^{64,65} and the binding strength between them and the TeNRs⁵⁸. Considering the dependencyof TeNRs growth on surfactants as driving force, the presence of amphiphilic molecules within TeNRs extract that might act as surfactant-like molecules, facilitating the 1D growth of the TeNRs, was evaluated with FCS exploiting the lipophilic tracer DiOC₁₈(3). The tracer does not emit fluorescence in aqueous solution⁶⁶, but its emission is enhanced when it is bound to a hydrophobic environment45. In this regard, FCS analysis was performed on the lipophilic tracer either dissolved in methanol or added to the aqueous extract containing TeNRs to evaluate the diffusion coefficients of the DiOC₁₈(3) tracer in different environments. FCS data showed a higher diffusion coefficient (D) value of DiOC₁₈(3) dissolved in methanol (D=345 μm² s⁻¹)⊐ as compared to the one obtained in the case of the TeNRs extract labeled with the lipophilic tracer (D=3.79 μm^{t} s⁻¹). Hence, the calculated DiOC₁₈(3) diffusion time (T_{Δ}) was lower in methanol (T_{Δ}=0.22 μs) than that in the context of the TeNRs extract (T_{Δ}=19.8 μs), indicating the amphiphilic nature of the molecules present within TeNRs extract, which slowed DiOC₁₈(3) diffusion. FCS results strongly suggested the presence of amphiphilic molecules within TeNRs extracts, which can both mediate NRs formation and stabilization. In this regard, Rhodococcus strains have been described for their ability to produce surfactant-like molecules (i.e., trehalose mycolates) under physiological conditions of growth⁶⁷. Thus, it results reasonable to suggest a possible surfactant-assisted growthof TeNRs within BCP1 cells.

Considering the crystalline nature of TeNRs within the extract, as well as semiconductive properties of tellurium²⁷, we explored the conductive properties of the biogenetically produced TeNRs, measuring their resistance (R) through the Four Probe technique⁴⁷. The TeNRs extract suspension air dried on the silicon support gave a low resistance value (R=8±1 Ω), as compared to the one of the silicon chip itself (R=281±7 0), and the material surrounding TeNRs (R=145±2 Ω), corresponding to an electrical conductivity (a) of 3.0±0.5, 0.08±0.002 and 0.16±0.02 S m⁻¹, respectively. Thus, TeNRs within the extract were able to reduce the resistance of the sample and, therefore, were shown to be electrically conductive, approaching the electrical conductivity values of those chemically synthesized, with a ranging between 8 and 10 S m^{-1 68,69}.

Additional details of materials and methods regarding bacterial cultures and TeO₃²⁻ -exposed cells, as well as TeO₃²⁻ bioconversion assays are described in this section. FIG. 29 represent control experiments to test abiotic loss of TeO₃²⁻ over the incubation time, while FIGS. 30-32 and FIGS. 33-35 show the complete Time Course performed by TEM of BCP1 resting cells exposure to the different initial concentration of TeO₃²⁻ and biogenic Te-nanostructure extracts production. FIG. 36 displays the length distribution of TeNRs produced by the BCP1 strain, considering the initial TeO₃²⁻ concentration and cellular exposure time.

### References

[1] Di Tommaso, G.; Fedi, S.; Carnevali, M.; Manegatti, M.; Taddei, C.; Zannoni, D. The membrane-bound respiratory chain of Pseudomonas pseudoalcaligenes KF707 cells grown in the presence or absence of potassium tellurite. Microbiology. 2002, 148, 1699-1708.

[2] Haynes, W. M. Section 4: properties of the elements and inorganic compounds. In CRC Handbook of Chemistry and Physics 95th ed., Haynes W. M.; Lide D. R.; Bruno, T. J., Eds; CRC Press/Taylor and Francis: Boca Raton, 2014, pp. 115-120.

[3] Cairnes, D. D. Canadian-containing ores. J. Can. Min. Inst. 1911, 14,185-202.

[4] Cooper W. C., Ed. Tellurium, Van Nostrand Reinhold Co: New York, 1971.

[5] Taylor, D. E. Bacterial tellurite resistance. Trends. Microbiol. 1999, 7,111-115.

[6] Turner, R. J. Tellurite toxicity and resistance in Gram-negative bacteria. Rec. Res. Dev. 352 Microbiol. 2001, 5,69-77.

[7] Harrison, J. J.; Ceri, H.; Stremick, C. A.; Turner, R. J. Biofilm susceptibility to metal toxicity. Environ. Microbiol. 2004, 6,1220-1227.

[8] Carmel-Harel, O.; Storz, G. Roles of the glutathione- and thioredoxin dependent reduction systems in the Escherichia coli and Saccharomyces cerevisiae responses to oxidative stress. Ann. Rev. Microbiol. 2000, 54,439-461.

[9] Held, K. D.; Biaglow, J. E. Mechanisms for the oxygen radical mediated toxicity of various thiol-containing compounds in cultured mammalian cells. Radiat. Res. 1994, 139,15-23.

[10] Morales, E. H.; Pinto, C. A.; Luraschi, R.; Munoz-Villagran, C. M.; Cornejo, F. A. S.; Simpkins, W.; Nelson, J.; Arenas, F. A.; Piotrowski, J. S.; Myers, C. L.; Mori, H.; Vasquez, C. C. Accumulation of heme biosynthetic intermediates contributes to the antibacterial action of the metalloid tellurite. Nat. Commun. 2017, 8,15320.

[11] Turner, R. J.; Borghese, R.; Zannoni, D. Microbial processing of tellurium as a tool in biotechnology. Biotechnol. Adv. 2012, 30,954-963.

[12] Borghese, R.; Borsetti, F.; Foladori, P.; Ziglio, G.; Zannoni, D. Effects of the Metalloid Oxyanion Tellurite (TeO32-) on growth characteristics of the phototrophic Bacterium Rhodobacter capsulatus. Appl. Environ. Microbiol. 2004, 70,6595-602.

[13] Borghese, R.; Brucale, M.; Fortunato, G.; Lanzi, M.; Mezzi, A.; Valle, F.; Cavallini M.; Zannoni, D. Extracellular Production of Tellurium Nanoparticles by the Photosynthetic Bacterium Rhodobacter capsulatus. J. Hazard. Mater. 2016, 309,202-209.

[14] Klonowska, A.; Heulin, T.; Vermeglio, A. Selenite and Tellurite Reduction by Shewanella oneidensis. Appl. Environ. Microbiol. 2005, 71,5607-5609.

[15] Amoozegar, M. A.; Ashengroph, M.; Malekzadeh, F.; Razavi, M. R.; Naddaf, S.; Kabiri, M. Isolation and initial characterization of the tellurite reducing moderately halophilic bacterium, Salinicoccus sp. strain QW6. Microbiol. Res. 2008, 163,456-465.

[16] Tucker, F. L.; Thomas, J. W.; Appleman, M. D.; Donohue, J. Complete reduction of tellurite to pure tellurium metal by microorganisms. J. Bacteriol. 1962, 83,1313-1314.

[17] Zare, B.; Faramarzi, M. A.; Sepehrizadeh, Z.; Shakibaie, M.; Rezaie, S.; Shahverdi, A. R. Biosynthesis and recovery of rod-shaped tellurium nanoparticles and their bactericidal activities. Mat. Res. Bull. 2012, 47,3719-3725.

[18] Morton, H. E.; Anderson, T. F. Electron microscopic studies of biological reactions. I. Reduction of potassium tellurite by Corynebacterium diphtheriae. Proc. Soc. Exptl. Biol. Med. 1941, 46,272-384 276.

[19] Terai, T.; Kamahora, Y.; Yamamura, Y. Tellurite reductase from Mycobacterium avium. J. Bacteriol. 1958, 75,535-539.

[20] Kim, D. H.; Kanaly, R. A.; Hur, H. G. Biological accumulation of tellurium nanorod structures via reduction of tellurite by Shewanella oneidensis MR-1. Bioresour. Technol. 2012, 125,127-131.

[21] Zonaro, E.; Lampis, S.; Turner, R. J.; Qazi, S. J. S.; Vallini, G. Biogenic selenium and tellurium nanoparticles synthetized by environmental microbial isolates efficaciously inhibit bacterial planktonic cultures and biofilms. Front. Microbiol. 2015, 6,584.

[22] Ingale, A. G.; Chaudhari, A. N. Biogenic Synthesis of Nanoparticles and Potential Applications: an EcoFriendly Approach. J. Nanomed. Nanotechnol. 2013, 4,165.

[23] Lampis, S.; Zonaro, E.; Bertolini, C.; Bernardi, P.; Butler, C. S.; Vallini G. Delayed formation of zero-valent selenium nanoparticles by Bacillus mycoides SelTE01 as a consequence of selenite reduction under aerobic conditions. Microb. Cell. Fact. 2014, 13,35.

[24] Zhao, A.; Zhang, L.; Yang, Y.; Ye, C. Ordered tellurium nanowire arrays and their optical properties. Appl. Phys. A. 2005, 80,1725-1728.

[25] Araki, K.; Tanaka, T. Piezoelectric and Elastic Properties of Single Crystalline Se—Te Alloys. Appl. Phys. Expr. 1972, 11,472-479.

[26] Tangney, P.; Fahy, S. Density-functional theory approach to ultrafast laser excitation of semiconductors: Application to the A1 phonon in tellurium. Phys Rev B. 2002, 14,279.

[27] Suchand Sandeep, C. S.; Samal, A. K.; Pradeep, T.; Philip, R. Optical limiting properties of Te and Ag2Te nanowires. Chem. Phys Lett. 2010, 485,326-330.

[28] Sharma, Y. C.; Purohit, A. Tellurium based thermoelectric materials: New directions and prospects. J. Integr. Sci. Technol. 2016, 4,29-32.

[29] Panahi-Kalamuei, M.; Mousavi-Kamazani, M.; Salavati-Niasari, M. Facile Hydrothermal Synthesis of Tellurium Nanostructures for Solar Cells. JNS. 2014, 4,459-465.

[30] Tsiulyanua, D.; Marian, S.; Miron, V.; Liess, H. D. High sensitive tellurium based NO2 gas sensor. Sens. Actuators B. 2001, 73,35-39.

[31] Baghchesara, M. A.; Yousefi, R.; Cheraghizadec, M.; Jamali-Sheinid, F.; Saáedi, A.; Mahmmoudiane, M. R. A simple method to fabricate an NIR detector by PbTe nanowires in a large scale. Mater. Res. Bull. 2016, 77,131-137.

[32] Huang, W.; Wu, H.; Li X.; Chen, T. Facile One-Pot Synthesis of Tellurium Nanorods as Antioxidant and Anticancer Agents. Chem. Asian J. 2016, 11,2301-2311.

[33] Martinková, L.; Uhnáková, B.; Pátek, M.; Nesvera, J.; Kren, V. Biodegradation potential of the genus Rhodococcus. Environ. Int. 2009, 35,162-177.

[34] Cappelletti, M.; Presentato, A.; Milazzo, G.; Turner, R. J.; Fedi, S.; Frascari, D.; Zannoni, D. Growth of Rhodococcus sp. strain BCP1 on gaseous n-alkanes: new metabolic insights and transcriptional analysis of two soluble di-iron monooxygenase genes. Front in Microbiol. 2015, 6,393.

[35] Frascari, D.; Pinelli, D.; Nocentini, M.; Fedi, S.; Pii, Y.; Zannoni, D. Chloroform degradation butane-grown cells of Rhodococcus aetherovorans BCP1. Appl Microbiol Biotechnol. 2006, 73,421-424 428.

[36] Cappelletti, M.; Fedi, S.; Frascari, D.; Ohtake, H.; Turner, R. J.; Zannoni, D. Analyses of both the alkB gene transcriptional start site and alkB promoter-inducing properties of Rhodococcus sp. strain BCP1 grown on n-alkanes. Appl. Environ. Microbiol. 2011, 77,1619-1627.

[37] Orro, A.; Cappelletti, M.; D'Ursi, P.; Milanesi, L.; Di Canito, A.; Zampolli, J.; Collina, E.; Decorosi, F.; Viti, C.; Fedi, S.; Presentato, A.; Zannoni D.; Di Gennaro, P. Genome and Phenotype Microarray Analyses of Rhodococcus sp. BCP1 and Rhodococcus opacus R7: Genetic Determinants and Metabolic Abilities with Environmental Relevance. PLoS ONE. 2015, 10,10.

[38] Cappelletti, M.; Fedi, S.; Zampolli, J.; Di Canito, A.; D'Ursi, P.; Orro, A.; Viti, C.; Milanesi, L.; Zannoni D.; Di Gennaro, P. Phenotype microarray analysis may unravel genetic determinants of the stress response by Rhodococcus aetherivorans BCP1 and Rhodococcus opacus R7. Res. Microbiol. 2016, 167,766-773.

[39] Ahmad, A.; Senapati, S.; Islam Khan, M.; Kumar, R.; Ramani, R.; Srinivas, V.; Sastry, M. Intracellular synthesis of gold nanoparticles by a novel alkalotolerant actinomycete, Rhodococcus species. Nanotechnol. 2003, 14,824-828.

[40] Otari, S. V.; Patil, R. M.; Nada, N. H.; Ghosh, S. J.; Pawar, S. H. Green biosynthesis of silver nanoparticles from an actinobacteria Rhodococcus sp. Mater Lett. 2012, 72,92-94.

[41] Kundu, D.; Hazra, C.; Chatterjee, A.; Chaudhari, A.; Mishra, S. Extracellular biosynthesis of zinc oxide nanoparticles using Rhodococcus pyridinivorans NT2: Multifunctional textile finishing, biosafety evaluation and in vitro drug delivery in colon carcinoma. J. Photochem. Photobiol. B. 2014, 140,194-204.

[42] Presentato, A.; Piacenza, E.; Anikovskiy, M.; Cappelletti, M.; Zannoni, D.; Turner, R. J. Rhodococcus aetherivorans BCP1 as Cell Factory for the Production of Intracellular Tellurium Nanorods under Aerobic Conditions. Micro. Cell Fact. 2016, 15,204.

[43] Turner, R. J.; Weiner, J. H.; Taylor, D. E. Use of Diethyldithiocarbamate for Quantitative Determination of Tellurite Uptake by Bacteria. Anal. Biochem. 1992, 204,292-295.

[44] Forootanfar, H.; Zare, B.; Fasihi-Bam, H.; Amirpour-Rostami, S.; Ameri, A.; Shakibaie, M. Biosynthesis and Characterization of Selenium Nanoparticles Produced by Terrestial Actinomycete Streptomyces microflavus Strain FSHJ31. JMB. 2014, 3,47-53.

[45] Yefimova, S. L.; Gural'chuk, G. Y.; Sorokin, A. V.; Malyukin, Y. V.; Borovoy, I. A.; Lubyanaya, A.S. Hydrophobicity effect of interaction between organic molecules in nanocages of surfactant micelle. J. Appl. Spectrosc. 2008, 75,658-663.

[46] Rigler, R.; Mets, U.; Widengren, J.; Kask, P. Fluorescence correlation spectroscopy with high count rate and low background: analysis of translational diffusion. Eur. Biophys. J. 1993, 22,169-458 175.

[47] Smits, F. M. Measurement of Sheet Resistivities with the Four-Point Probe. Bell Labs Tech. J. 1958, 37,711-718.

[48] Singh, P.; Kim, Y. J.; Zhang, D.; Yang, D. C. Biological Synthesis of Nanoparticles from Plants and Microorganisms. Trends Biotechnol. 2016, 34,588-599.

[49] Wang, X.; Liu, G.; Zhou, J.; Wang, J.; Jin, R.; Lv, H. Quinone-mediated reduction of selenite and tellurite by Escherichia coli. Bioresour. Technol. 2011, 102,3268-3271.

[50] Baesman, S. M.; Bullen, T. D.; Dewald, J.; Zhang, D.; Curran, S.; Islam, F. S.; Beveridge T. J.; Oremland, R. S. Formation of tellurium nanocrystals during anaerobic growth of bacteria that use Te oxyanions as respiratory electron acceptors. Appl. Environ. Microbiol. 2007, 73,2135-2143.

[51] Kim, D H.; Kanaly, R A.; Hur, H G. Biological accumulation of tellurium nanorod structures via reduction of tellurite by Shewanella oneidensis MR-1. Bioresour. Technol. 2012, 125,127-131.

[52] Berger, L. I. Chapter 3: Elemental Semiconductors. In Semiconductor Materials, Berger L. I., Ed; CRC Press: Boca Raton, 1997, pp. 88-90.

[53] Xi, B.; Xiong, S.; Fan, H.; Wang, X.; Qian, Y. Shape-Controlled Synthesis of Tellurium 1D Nanostructures via a Novel Circular Transformation Mechanism. Cryst. Growth Des. 2007, 7,1185-1191.

[54] Mayers, B.; Xia, Y. One-dimensional nanostructures of trigonal tellurium with various morphologies can be synthesized using a solution-phase approach. J. Mater. Chem. 2002, 12,1875-1881.

[55] Mo, M.; Zeng, J.; Liu, X.; Yu, W.; Zhang, S.; Quian, Y. Controlled Hydrothermal Synthesis of Thin Single-Crystal Tellurium Nanobelts and Nanotubes. Adv. Mater. 2002, 14,1658-1662.

[56] Li, X. L.; Cao, G. H.; Feng, C. M.; Li, Y. D. Synthesis and magnetoresistance measurement of tellurium microtubes. J. Mater, Chem. 2004, 14,244-247.

[57] Gautam, U. K.; Rao, C. N. R. Controlled synthesis of crystalline tellurium nanorods, nanowires, nanobelts and related structures by a self-seeding solution process. J. Mater. Chem. 2004, 14,2530-2535 (2004).

[58] Liu, Z.; Hu, Z.; Liang, J.; Li, S.; Yang, Y.; Peng S.; Qian, Y. Size-Controlled Synthesis and Growth Mechanism of Monodisperse Tellurium Nanorods by a Surfactant-Assisted Method. Langmiur. 2004, 20,214-218.

[59] Nikoobakht, B.; El-Sayed, M. A. Preparation and Growth Mechanism of Gold Nanorods (NRs) Using Seed-Mediated Growth Method. Chem. Mater. 2003, 15,1957-1962 (2003).

[60] Jana, N. R.; Gearheart, L.; Murphy, C. J. Wet chemical synthesis of silver nanorods and nanowires of controllable aspect ratio. Chem. Commun. 2001, 617-618.

[61] Puntes, V. F.; Krishnan, K. M.; Alivisatos, A. P. Colloidal nanocrystal shape and size control: the case of cobalt. Science. 2001, 291,2115-2117.

[62] Cao, G. S.; Zhang, X. J.; Su, L.; Ruan, Y. Y. Hydrothermal synthesis of selenium and tellurium nanorods. J. Exp. Nanosci. 2011, 6,121-126.

[63] Zhu, Y.; et al. Chapter 7: Hydrothermal synthesis of Inorganic Nanomaterials. In Nanorods,Nanotubes and Nanomaterials Research Progress; Prescott, W. V., Schwartz, A. I., Eds. Nova Science Publishers: New York, 2008, pp. 279-304.

[64] Rosen, M. J. Chapter 1: Characteristic Features of Surfactants. In Surfactants and Interfacial Phenomena 3rd ed.; Rosen, M. J., Ed. Wiley-Interscience: Hoboken, 2004, pp. 1-33.

[65] Zhu, H.; Zhang, H.; Liang, J.; Rao, G.; Li, J.; Liu, G.; Du, Z.; Fan H.; Luo, J. Controlled synthesis of tellurium nanostructures from nanotubes to nanorods and nanowires and their template applications. J. Phys. Chem. C. 2011, 115,6375-6380.

[66] Hauglang, R. P., Ed. Handbook of Fluorescent Probes and Research Products, 9th Ed.; Molecular Probes: Eugene, 2002.

[67] Rapp, P.; Bock, H.; Wray, V.; Wagner, F. Formation, isolation and characterization of trehalose dimycolates from Rhodococcus erythropolis grown on n-alkanes. J. Gen. Microbiol. 1979, 115,491-503.

[68] See, K. C.; Feser, J. P.; Chen, C. E.; Majumdar, A.; Urban, J. J.; Segalman, R. A. Water-Processable Polymer-Nanocrystal Hybrids for Thermoelectrics. Nano Lett. 2010, 10,4664-4667.

[69] Yee, S. K.; Coates, N. E.; Majumdar, A.; Urban, J. J.; Segalman, R. A. Thermoelectric power factor optimization in PEDOT:PSS tellurium nanowire hybrid composites. Phys. Chem. Chem. Phys.2013, 15, 4024-4032.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

What is claimed is:

1. A method of producing tellurium nanostructures, comprising: culturing Rhodococcus aetherivorans (BCP1) bacteria in a medium comprising tellurite.

2. The method of claim 1, wherein said culturing comprises pre-culturing said bacteria in said medium to generate a pre-culture, followed by culturing a portion of said pre-culture in said medium comprising tellurite to form a first culture.

3. The method of claim 1 or 2 further comprising a culturing a portion of said first culture in said medium comprising tellurite to form a second culture.

4. The method of one of claims 1 to 3, wherein said culturing is performed under aerobic conditions.

5. The method of any one of claims 1 to 4, wherein said culturing is performed under aerobic conditions at temperatures 20-40° C..

6. The method of any one of claims 1 to 5, wherein said tellurite comprises TeO₃²⁻, HTeO₃⁻, H₂TeO₃²⁻, K₂TeO₃, or Na₂TeO₃.

7. The method of any one of claims 1 to 6, wherein the concentration of said tellurite is between about 0.4 mM (100 μg/ml) to about 2 mM (500 μg/ml).

8. The method of one of claims 1 to 7, wherein said tellurium nanostructures are formed in the shape of uniform nanorods or and not crystals.

9. The method of any one of claims 1 to 8, wherein said tellurium nanostructures are formed in the shape of uniform spherical nanoparticles.

10. The method of any one of claims 1 to 9, wherein said tellurium nanostructures that are formed are stable, dispersed and non-aggregated.

11. The method of any one of claims 1 to 10, wherein said tellurium nanorods have a length of about 100 nm to about 1000 nm.

12. The method of any one of claims 1 to 11, further comprising isolating said produced tellurium nanostructures.

13. The method of claim 12, wherein said isolating comprises collecting said BCP1 cells, washing said collected BCP1 cells, disrupting said collected BCP1 cells, and extracting said tellurium nanostructures from said disrupted BCP1 cells.

14. The method of claim 13, wherein said collecting of said BCP1 cells comprises centrifugation.

15. The method of claim 13 or 14, wherein said washing of said collected BCP1 cells comprises washing with a saline solution.

16. The method of any one of claims 13 to 15, wherein said disrupting comprises sonication.

17. The method of any one of claims 13 to 16, wherein said extracting of said tellurium nanostructures comprises removing the cellular debris following said disrupted cells to obtain a supernatant, and isolating the tellurium nanostructures from said supernatant.

18. A tellurium nanorod produced according to any one of claims 1 to 17.

19. A tellurium nanorod produced according to any one of claims 1 to 17 for use in:
a. electronics or electronics equipment,
b. glass or industrial glass,
c. as alloys, preferably with copper, cadmium or stainless steel,
d. batteries as an anti-corrosive or semiconductor
e. ceramic as a colouring agent,
f. photosensitive semiconductors, optics, quantum dots.
g. a thin film in solar panels,
h. in catalysts for petroleum cracking and in blasting caps for explosives,
i. petroleum refining, or
j. mining.
k. antifouling coatings,
l. antioxidant agents,
m. human and agricultural pharmaceuticals: antimicrobials, biocides, antifungals, antivirals, anticancer agents,
n. piezoelectric devices.

20. A method of producing selenium nanostructures, comprising: culturing Rhodococcus aetherivorans (BCP1) bacteria in a medium comprising selenium.

21. The method of claim 20, wherein said culturing comprises pre-culturing said bacteria in said medium to generate a pre-culture, followed by culturing a portion of said pre-culture in said medium comprising selenium to form a first culture.

22. The method of claim 20 or 21 further comprising a culturing a portion of said first culture in said medium comprising selenium to form a second culture.

23. The method of any one of claims 20 to 22, wherein said culturing is performed under aaerobic conditions.

24. The method of any one of preceding claims 20 to 23, wherein said culturing is performed under aerobic conditions at about 20-40° C.

25. The method of any one of claims 20 to 24, wherein said selenium comprises SeO₃²⁻, HSeO₃⁻, H₂SeO₃²⁻, K₂SeO₃ Na₂SeO₃, or Na₂SeO₄.

26. The method of any one of claims 20 to 25, wherein the concentration of said selenium is between about 0.5 mM to >200 mM , preferably 0.5 mM to 200 mM.

27. The method of any one of claims 20 to 26, wherein said selenium nanostructures are formed in the shape of uniform spherical nanoparticles or nanorods and not crystals.

28. The method of any preceding claim, wherein said selenium nanostructures that are formed are stable, dispersed and non-aggregated.

29. The method of any one of claims 20 to 27, wherein said selenium nanoparticles have a diameter of about 50 nm to about 250 nm.

30. The method of any one of claims 20 to 27, wherein said nanorods have a length of about 20 nm to about 1000 nm.

31. The method of any one of claims 20 to 30, further comprising isolating said produced selenium nanostructures.

32. The method of claim 31, wherein said isolating comprises collecting said BCP1 cells, washing said collected BCP1 cells, disrupting said collected cell, and extracting said selenium nanostructures from said washed BCP1 cells.

33. The method of claim 32, wherein said collecting of said BCP1 cells comprises centrifugation.

34. The method of claim 32 or 33, wherein said washing of said collected BCP1 cells comprises washing with a saline solution.

35. The method of any one or claims 32 to 34, wherein said extracting of said selenium nanostructures comprises removing the cellular debris following said disrupted cells to obtain a supernatant, and isolating the selenium nanostructures from said supernatant.

36. A selenium nanorod or nanoparticle produced according to any one of claims 20 to 35.

37. A selenium nanorod or nanoparticle produced according to any one of claims 20 to 36 for use in:
a. electronics or electronics equipment,
b. glass or industrial glass,
c. animal feed,
d. food supplements,
e. as alloys, preferable an alloy for batteries
f. production of pigments, or
g. production of plastics.
h. optics
i. production of medical devices.
j. antifouling coatings,
k. antioxidant agents,
l. human and agricultural pharmaceuticals: antimicrobials, biocides, antifungals, antivirals, anticancer agents,
m. quantum dots.

38. A nanorod produced according to the method of any one of claims 1 to 37, wherein said nanorod is a nanoribbon (flat structure), nanotube (hollow structure) or solid nanorod.

39. An electronic device comprising: a substrate and one or more tellurium nanorods forming an electrically conductive path in said substrate.

40. The electronic device of claim 39, wherein said one or more tellurium nanorods are made according to the method of any one of 1 to 17.

41. An electrically conductive material comprising: a substrate and one or more tellurium nanorods forming an electrically conductive path in said substrate.

42. The electrically conductive material of claim 41, wherein said one or more tellurium nanorods are made according to the method of any one of claims 1 to 17.

43. An electric device comprising an electrically conductive material of claim 41 or 42, wherein said electronic device is a resistor, capacitor, support, semiconductor, or wire.

44. An electronic device comprising: a substrate and one or more selenium nanorods forming an electrically conductive path in said substrate.

45. The electronic device of claim 44, wherein said one or more selenium nanorods are made according to any one of claims 20 to 35.

46. An electrically conductive material comprising: a substrate and one or more selenium nanorods forming an electrically conductive path in said substrate.

47. The electrically conductive material according to claim 46, wherein said one or more selenium nanorods are made according to any one of claims 20 to 35.

48. An electric device comprising an electrically conductive material of claim 46 or 47, wherein said electronic device is a resistor, capacitor, support, semiconductor, or wire.
